(19) United States
(12) Patent Application Publication
(10) Pub. No.: US 2010/0292185 A1
(43) Pub. Date: Nov. 18, 2010

(54) BETA-LACTAMASE INHIBITORS

(76) Inventors: Christopher J. Burns, Malvern, PA (US); Rajesh Goswami, Secane, PA (US); Randy W. Jackson, Glenmore, PA (US); Thomas Lessen, Langhorne, PA (US); Weiping Li, Norristown, PA (US); Daniel Pevear, Downingtown, PA (US); Pavan Kumar Tirunahari, Exton, PA (US); Hongyu Xu, Phoenixville, PA (US)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/776,745

(22) Filed: May 10, 2010

Related U.S. Application Data
(60) Provisional application No. 61/177,488, filed on May 12, 2009.
(60) Provisional application No. 61/177,481, filed on May 12, 2009.
(60) Provisional application No. 61/289,232, filed on Dec. 22, 2009.

(51) Int. Cl. A61K 31/69; C07F 5/02; A61P 31/00

(52) U.S. Cl. 514/64; 562/7; 544/69; 544/229; 546/13; 548/110

(57) ABSTRACT

Disclosed herein are α-aminoboronic acids and their derivatives which act as inhibitors of beta-lactamases. Also disclosed herein are pharmaceutical compositions comprising α-aminoboronic acids and methods of use thereof.

### FIELD OF THE INVENTION

The present disclosure relates to α-aminoboronic acids and their derivatives which act as inhibitors of beta-lactamase enzymes.

### BACKGROUND OF THE INVENTION

Antibiotics are the most effective drugs for curing bacteria-infectious diseases clinically. They have a wide market for their advantages of good antibacterial effect, and limited side effect. Among them, beta-lactam antibiotics (for example, penicillins, cephalosporins, and carbapenems) are widely used because they have a very strong bactericidal effect (by blocking cell division) and very low toxicity.

To counter the efficacy of the various beta-lactams, bacteria have evolved to produce variants of beta-lactam deactivating enzymes called beta-lactamases, and in the ability to share this tool inter- and intra-species. The rapid spread of this mechanism of bacterial resistance can severely limit beta-lactam treatment options in the hospital and in the community. Beta-lactamases are typically grouped into 4 classes: Ambler classes A, B, C, and D, based on their amino acid sequences. Enzymes in classes A, C, and D are active-site serine beta-lactamases, while class B enzymes, which are encountered less frequently, are Zn-dependent. Newer generation cephalosporins and carbapenems were developed partly based on their ability to evade the deactivating effect of the early serine-based beta-lactamase variants. However, a recent surge in new versions of serine-based beta-lactamases—for example Class A Extended-Spectrum Beta-Lactamase (ESBL) enzymes, Class A carbapenemases (e.g. KPC-2), chromosomal and plasmid mediated Class C cephalosporinases (AmpC, CMY, etc.), and Class D oxacillinases—has begun to diminish the utility of the beta-lactam antibiotic family, including the more recent generation beta-lactam drugs, leading to a serious medical problem. Indeed the number of catalogued serine-based beta-lactamases has exploded from less than ten in the 1970s to over 300 variants (see, e.g., Jacoby & Bush, “Amino Acid Sequences for TEM, SHV and OXA Extended-Spectrum and Inhibitor Resistant β-Lactamases”, on the Lahey Clinic website).

The commercially available beta-lactamase inhibitors (clavulanic acid, sulbactam, tazobactam) were developed to address the beta-lactamases that were clinically relevant in the 1970s and 1980s (e.g. penicillinases). These enzyme inhibitors are available only as fixed combinations with penicillin derivatives. No combinations with cephalosporins (or carbapenems) have been developed or are clinically available. This fact, combined with the increased use of newer generation cephalosporins and carbapenems, is driving the selection and spread of the new beta-lactamase variants (ESBLs, carbapenemases, chromosomal and plasmid-mediated class C, class D oxacillinases, etc.). While maintaining good inhibitory activity against ESBLs, the legacy beta-lactamase inhibitors are largely ineffective against the new Class A carbapenemases, against the chromosomal and plasmid-mediated Class C cephalosporinases and against many of the Class D oxacillinases. To address this growing therapeutic vulnerability, a new generation of beta-lactamase inhibitors must be developed with broad spectrum functionality. The novel boronic acid based inhibitors described herein address this medical need.

Use of a boronic acid compound to inhibit a beta-lactamase enzyme has been limited. For example, U.S. Pat. No. 7,271,186 discloses beta-lactamase inhibitors that target AmpC (from class C). Ness et al. (Biochemistry (2000) 39:5312-21) discloses beta-lactamase inhibitors that target TEM-1 (a non-ESBL TEM variant from class A; one of approximately 140 known TEM-type beta-lactamase variants). Because there are three major molecular classes of serine-based beta-lactamases, and each of these classes contain significant numbers of beta-lactamase variants, inhibition of one or a small number of beta-lactamases is unlikely to be of therapeutic value. Therefore, there is an imperative need to develop novel beta-lactamase inhibitors with broad spectrum functionality.

### SUMMARY OF THE INVENTION

One aspect is for a compound of Formula (I):

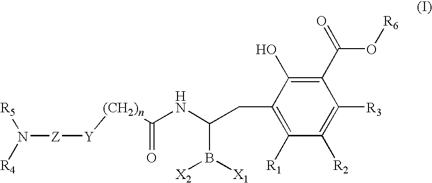

wherein R₁, R₂, and R₃ are independently hydrogen, or selected from the group consisting of hydroxyl, halogen, carboxyl, cyano, thiol, optionally substituted: C1-C5 alkyl, C1-C5 alkoxy, C1-C5 alkenyl, C3-C6 cycloalkyl, C3-C6 heterocyclyl, amino, sulfide, and sulfone;
n is 0, 1, or 2;
Y is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(c) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
   R₄ is hydrogen, or selected from the group consisting of:
(a) C1-C5 alkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C1-C5 carbons comprise part of said oxyimino group, imino wherein any of the C1-C5 carbons comprise part of said imino group, amidino wherein any of the C1-C5 carbons comprise part of said amidino group, sulfido, and sulfoxido,
(b) C3-C6 cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfido, and sulfoxido,
(c) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
   R₅ is a lone pair of electrons, hydrogen, or selected from the group consisting of:
(a) C1-C5 alkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C1-C5 carbons comprise part of said oxyimino group, imino wherein any of the C1-C5 carbons comprise part of said imino group, amidino wherein any of the C1-C5 carbons comprise part of said amidino group, sulfido, and sulfoxido,
(b) C3-C6 cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfido, and sulfoxido,
(c) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
   or R₄ and Y together form a ring of between 5 and 7 atoms where said ring is optionally fused or spiro in relation to the ring system of Y, said ring optionally being partially saturated or aromatic and optionally containing 1-2 additional heteroatoms selected from the group consisting of N, O, S, and a combination thereof;
   or R₄ and R₅ together form a ring of between 3 and 7 atoms where said ring is optionally substituted, said ring optionally being saturated, partially unsaturated or aromatic and optionally containing 1-2 additional heteroatoms selected from the group consisting of N, O, S, and a combination thereof;
   R₆ is hydrogen or an ester prodrug of the carboxylic acid;
   Z is a bond;
   or Z is optionally substituted: C1-C4 alkyl, C1-C4 alkoxy, C1-C4 sulfido, C3-C6 cycloalkyl, C3-C6 heterocyclyl where the bond to Y is through a carbon atom of said heterocyclyl ring, heteroaryl where the bond to Y is through a carbon atom of said heteraryl ring, oxyimino, imino, or amidino where the carbon of said oxyimino, imino, or amidino group is attached to Y;
   or Z and Y together form a ring of 5-7 atoms where said ring is optionally fused or spiro in relation to the ring system of Y, said ring optionally being partially saturated or aromatic and optionally containing 1-3 heteroatoms selected from the group consisting of N, O, S, and a combination thereof;
   or Z and R₄ together form a ring of 4-7 atoms where said ring optionally is saturated, partially unsaturated, or aromatic and optionally contains 1-2 additional heteroatoms selected from the group consisting of N, O, S, and a combination thereof;
   X₁ and X₂ are independently hydroxyl, halogen, NR₄R₅, C1-C6 alkoxy, or when taken together X₁ and X₂ form a cyclic boron ester where said chain or ring contains from 2 to 20 carbon atoms and, optionally, 1-3 heteroatoms selected from the group consisting of N, O, S and a combination thereof, or when taken together X₁ and X₂ form a cyclic boron amide where said chain or ring contains from 2 to 20 carbon atoms and, optionally, 1-3 heteroatoms selected from the group consisting of N, O, S, and a combination thereof, or when taken together X₁ and X₂ form a cyclic boron amide-ester where said chain contains from 2-20 carbon atoms and, optionally, 1-3 heteroatoms selected from the group consisting of N, O, S, and a combination thereof, or X₁ is hydroxyl and X₂ is replaced by the ortho-hydroxyloxygen of the phenyl ring such that a 6-membered ring is formed;
   or a salt thereof;
   provided that when R₁, R₂, R₃, R₄, R₅, and R₆ are hydrogen, X₁ and X₂ are hydroxyl, n is 0, Y is phenyl, and Z is CH₂ then Z cannot be at the meta-position of the phenyl ring relative to the rest of the molecule.

Another aspect is for a pharmaceutical composition comprising: (a) one or more compounds discussed above; (b) one or more β-lactam antibiotics; and (c) one or more pharmaceutically acceptable carriers.

A further aspect is for a pharmaceutical composition comprising: (a) one or more compounds discussed above; and (b) one or more pharmaceutically acceptable carriers.

An additional aspect is for a method of treating a bacterial infection in a mammal comprising administering to a mammal in need thereof:

(i) an effective amount of a compound described above; and

(ii) an effective amount of a β-lactam antibiotic.

Another aspect is for a method of treating a bacterial infection in a mammal comprising administering to a mammal in need thereof an effective amount of a compound described above.

A further aspect is for a method of reducing bacterial resistance to a β-lactam antibiotic comprising contacting a bacterial cell having resistance to a β-lactam antibiotic with an effective amount of a beta-lactamase inhibitor with broad-spectrum functionality having the formula described above.

An additional aspect is for use of a beta-lactamase inhibitor with broad-spectrum functionality having the formula described above in combination with a β-lactam antibiotic in the manufacture of a medicament for the treatment of a bacterial infection.

Another aspect is for a composition for use in combination with a β-lactam antibiotic in reducing a bacterial infection, said composition being described above.

Other objects and advantages will become apparent to those skilled in the art upon reference to the detailed description that hereinafter follows.

### BRIEF DESCRIPTION OF THE FIGURES

FIG. 1. General synthetic scheme for the synthesis of α-amidoboronic acids using a tert-butyl ester derived from 3-borono-2-methoxybenzoic acid.

FIG. 2. General synthetic scheme for the synthesis of aminomethylbenzamide compounds derived from substituted benzamides.

FIG. 3. General synthetic scheme for the synthesis of α-amidoboronic acids from substituted salicylic acids and a trialkylborate.

FIG. 4. General synthetic scheme for the synthesis of α-amidoboronic acids from substituted 3-bromosalicylic acids and (+)-pinanediol-(bromomethyl)boronate.

FIG. 5. General synthetic scheme for the synthesis of α-amidoboronic acids from substituted 3-methylsalicylic acids.

FIG. 6. Equilibrium between the boronic acid open chain form and the boronic ester cyclic form of compounds possessing an ortho-phenol group.

FIG. 7. Structure of three beta-lactam antibiotics, PZ-601, ME1036, and BAL30072.

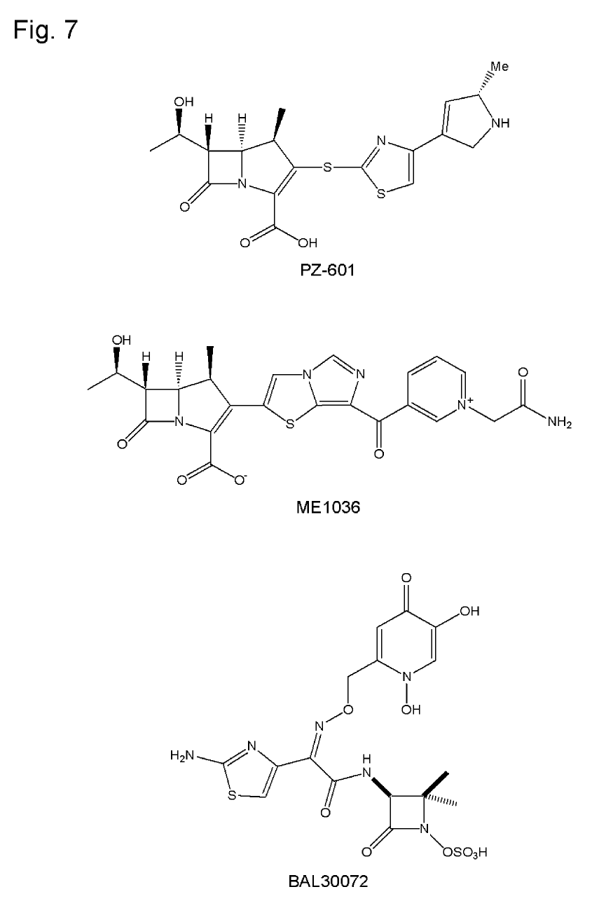

FIG. 8. General synthetic approaches for the synthesis of ester prodrugs of beta-lactamase inhibitors.

### DETAILED DESCRIPTION OF THE INVENTION

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The present invention relates generally to novel α-aminoboronic acids and their derivatives which act as broad-spectrum inhibitors of beta-lactamase enzymes. Beta-lactamases hydrolyze beta-lactam antibiotics, and are therefore an important cause of β-lactam antibiotic resistance. The compounds of the recent invention, particularly when administered in combination with a β-lactam antibiotic, overcome this resistance mechanism and render beta-lactamase producing bacteria susceptible to the β-lactam antibiotic. The present invention also relates to pharmaceutical compositions comprising a compound of the present invention, or salt thereof, an optional beta-lactam antibiotic, and a pharmaceutically acceptable excipient. The present invention also relates to a method for treating a bacterial infection in a mammal by administration of a therapeutically acceptable amount of the aforementioned pharmaceutical compositions. The present invention also relates to a method for increasing the effectiveness of a beta-lactam antibiotic in mammals by administering an effective amount of a compound of the present invention in combination with an effective amount of such beta-lactam antibiotic.

### DEFINITIONS

In the context of this disclosure, a number of terms shall be utilized.

As used herein, the term “about” or “approximately” means within 20%, preferably within 10%, and more preferably within 5% of a given value or range.

The term “antibiotic” is used herein to describe a compound or composition which decreases the viability of a microorganism, or which inhibits the growth or reproduction of a microorganism. “Inhibits the growth or reproduction” means increasing the generation cycle time by at least 2-fold, preferably at least 10-fold, more preferably at least 100-fold, and most preferably indefinitely, as in total cell death. As used in this disclosure, an antibiotic is further intended to include an antimicrobial, bacteriostatic, or bactericidal agent. Non-limiting examples of antibiotics useful according to this aspect of the invention include penicillins, cephalosporins, aminoglycosides, sulfonamides, macrolides, tetracyclins, lincosides, quinolones, chloramphenicol, vancomycin, metronidazole, rifampin, isoniazid, spectinomycin, trimethoprim, sulfamethoxazole, and others.

The term “beta-lactam antibiotic” is used to designate compounds with antibiotic properties containing a beta-lactam functionality. Non-limiting examples of beta-lactam antibiotics useful according to this aspect of the invention include penicillins, cephalosporins, penems, carbapenems, and monobactams. Beta-lactam antibiotics are effective (in the absence of resistance) against a wide range of bacterial infections. These include those caused by both gram-positive and gram-negative bacteria, for example, bacteria of the genus Staphylococcus (such as Staphylococcus aureus and Staphylococcus epidermidis), Streptococcus (such as Streptococcus agalactine, Streptococcus pneumoniae and Streptococcus faecalis), Micrococcus (such as Micrococcus luteus), Bacillus (such as Bacillus subtilis), Listerella (such as Listerella monocytogenes), Escherichia (such as Escherichia coli), Klebsiella (such as Klebsiella pneumoniae), Proteus (such as Proteus mirabilis and Proteus vulgaris), Salmonella (such as Salmonella typhosa), Shigella (such as Shigella sonnei), Enterobacter (such as Enterobacter aerogenes and Enterobacter cloacae), Serratia (such as Serratia marcescens), Pseudomonas (such as Pseudomonas aeruginosa), Acinetobacter (such as Acinetobacter anitratus), Nocardia (such as Nocardia autotrophica), and Mycobacterium (such as Mycobacterium fortuitum).

The term “beta-lactamase” means an enzyme produced by a bacteria that has the ability to hydrolyze the beta-lactam ring of beta-lactam antibiotics. Such enzymes are often classified into 4 major classes (Classes A, B, C, and D) according to the so-called Ambler classification scheme, based principally on protein homology.

The term “beta-lactamase inhibitors with broad-spectrum functionality” as used herein refers to the ability of an inhibitor to inhibit a broad range of beta-lactamase enzymes, spanning multiple subtypes from multiple classes (for example numerous enzyme subtypes from both Ambler Class A and Ambler Class C). In some embodiments, beta-lactamase enzyme(s) from at least two classes of beta-lactamase enzymes are inhibited by a compound disclosed herein, with preferred embodiments being those where beta-lactamase enzyme(s) from more than two classes of beta-lactamase enzymes are inhibited by a compound disclosed herein.

The term “comprising” is intended to include embodiments encompassed by the terms “consisting essentially of” and “consisting of”. Similarly, the term “consisting essentially of” is intended to include embodiments encompassed by the term “consisting of”.

The terms “effective amount”, “therapeutically effective amount”, and “therapeutically effective period of time” are used to denote known treatments at dosages and for periods of time effective to show a meaningful patient benefit, i.e., healing of conditions associated with bacterial infection, and/or bacterial drug resistance. Preferably, such administration should be parenteral, oral, sublingual, transdermal, topical, intranasal, intratracheal, or intrarectal. When administered systemically, the therapeutic composition is preferably administered at a sufficient dosage to attain a blood level of inhibitor of at least about 100 μg/mL, more preferably about 1 mg/mL, and still more preferably about 10 mg/mL. For localized administration, much lower concentrations than this may be effective, and much higher concentrations may be tolerated.

The term “mammal” refers to a human, a non-human primate, canine, feline, bovine, ovine, porcine, murine, or other veterinary or laboratory mammal. Those skilled in the art recognize that a therapy which reduces the severity of a pathology in one species of mammal is predictive of the effect of the therapy on another species of mammal.

### Chemical Definitions

The term alkyl means both straight and branched chain alkyl moieties of 1-12 carbons, preferably of 1-8 carbon atoms.

The term alkenyl means both straight and branched alkenyl moieties of 2-8 carbon atoms containing at least one double bond, and no triple bond, preferably the alkenyl moiety has one or two double bonds. Such alkenyl moieties may exist in the E or Z conformations; the compounds of this invention include both conformations.

The term alkynyl includes both straight chain and branched alkynyl moieties containing 2-6 carbon atoms containing at least one triple bond, preferably the alkynyl moiety has one or two triple bonds.

The term cycloalkyl refers to an alicyclic hydrocarbon group having 3-7 carbon atoms.

The term halogen is defined as Cl, Br, F, and I.

Aryl is defined as an aromatic hydrocarbon moiety selected from the group: phenyl, α-naphthyl, β-naphthyl, biphenyl, anthryl, tetrahydronaphthyl, fluorenyl, indanyl, biphenylenyl, or acenaphthenyl.

Heteroaryl is defined as an aromatic heterocyclic ring system (monocyclic or bicyclic) where the heteroaryl moieties are selected from, but not limited to: (1) furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, 1,2,4-thiadiazole, 1,3,4-thiadiazole, 1,2,3-thiadiazole, 1,2,3-triazole, 1-methyl-1,2,3-triazole, benzoxazole, benzothiazole, benzofuran, benzisoxazole, benzimidazole, N-methylbenzimidazole, azabenzimidazole, indazole, quinazoline, quinoline, and isoquinoline; (2) a bicyclic aromatic heterocycle where a phenyl, pyridine, pyrimidine or pyridizine ring is: (a) fused to a 6-membered aromatic (unsaturated) heterocyclic ring having one nitrogen atom; (b) fused to a 5 or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S.

Heterocyclyl is defined as a saturated or partially saturated heterocyclic moiety selected from, but not limited to: aziridinyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothienyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dihydro-1,4-dioxanyl, tetrahydrofuranyl, tetrahydrothienyl, tetrahydroquinolinyl, and tetrahydroisoquinolinyl.

Alkoxy is defined as C1-C6alkyl-O—.

Cycloalkoxy is defined as C3-C7cycloalkyl-O—.

Heteroaryloxy is defined as heteroaryl-O—.

Heterocyclyloxy is defined as C3-C7heterocyclyl-O—.

Sulfonic acid is defined as —SO₃H.

Sulfate is defined as —OSO₃H.

Amino is defined as —NH₂.

Cyano is defined as —CN.

Hydroxyl is defined as —OH.

Thiol is defined as —SH.

Carboxyl is defined as —CO₂H.

Oxo is defined as double bonded oxygen.

Trialkylammonium is defined as (A1)(A2)(A3)N⁺— where A1, A2 and A3 are independently alkyl, cycloalkyl, heterocyclyl and the nitrogen is positively charged.

Carbonyl is defined as —C(O)— where the carbon is optionally substituted and also attached to the rest of the molecule.

Aminocarbonyl is defined as —C(O)—N—, where the carbon is optionally substituted and the nitrogen is attached to the rest of the molecule.

Oxycarbonyl is defined as —C(O)—O—, where the carbon is optionally substituted and the oxygen is attached to the rest of the molecule.

Aminosulfonyl is defined as —S(O)₂—N— where the sulfur is optionally substituted and the nitrogen is attached to the rest of the molecule.

Sulfonyl is defined as —S(O)₂— where the sulfur is bonded to an optional substituent and also to the rest of the molecule.

Guanidino is defined as —N1-C(═N2)-N3- where N1, N2, and N3 are optionally substituted and N3, or N1 and N3 is attached to the rest of the molecule.

Oxyimino is defined as (═N—O-A) where the nitrogen is double bonded to a carbon which is attached to the rest of the molecule and A can be hydrogen, or optionally substituted: alkyl, cycloalkyl, aryl, heteroaryl, heterocyclyl

Imino is defined as (═N-A) where the nitrogen is double bonded to a carbon which is attached to the rest of the molecule and A can be hydrogen, or optionally substituted: alkyl, cycloalkyl, aryl, heteroaryl, heterocyclyl.

Amidino is defined as —C(═N1)-N2— where the carbon, N1 and N2 are optionally substituted and the carbon, or N2, or the carbon and N2 is attached to the rest of the molecule.

Sulfido is defined as —S— where sulfur is bound to an optional substituent and also to the rest of the molecule.

Sulfoxido is defined as —S(O)— where sulfur is bound to an optional substituent and also to the rest of the molecule.

Where a group or atom is described as “optionally substituted” one or more of the following substituents may be present on that group or atom: hydroxyl, halogen, carboxyl, cyano, thiol, amino, imino, oxyimino, amidino, guanidino, sulfonic acid, sulfate, alkyl, cycloalkyl, alkoxy, alkenyl, alkynyl, aryl, heteroaryl, heterocyclyl, cycloalkoxy, heterocyclyloxy, aryloxy, heteroaryloxy, arylakyl, alkylaryl, heteroarylalkyl, alkylheteroaryl, trialkylammonium, carbonyl, oxycarbonyl, aminocarbonyl. Optional substituents may be attached to the group or atom which they substitute in a variety of ways, either directly or through a connecting group of which the following are examples: alkyl, amine, amide, ester, ether, thioether, sulfonamide, sulfamide, sulfoxide, urea. As appropriate an optional substituent may itself be further substituted by another substituent, the latter being connected directly to the former or through a connecting group such as those exemplified above.

### Beta-Lactamase Inhibitors

The present disclosure relates to compounds of formula I. Certain compounds of Formula (I) include compounds of Formula (II):

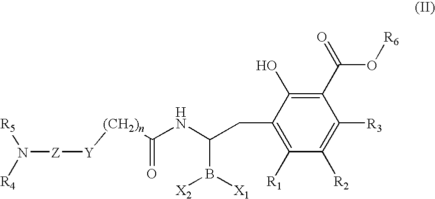

wherein R₁, R₂, and R₃ are independently hydrogen, or selected from the group consisting of hydroxyl, halogen, carboxyl, cyano, thiol, optionally substituted: C1-C5 alkyl, C1-C5 alkoxy, C1-C5 alkenyl, C3-C6 cycloalkyl, C3-C6 heterocyclyl, amino, sulfide, and sulfone;
n is 0, 1, or 2;
Y is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(c) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
   R₄ is hydrogen, or selected from the group consisting of:
(a) C1-C5 alkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C1-C5 carbons comprise part of said oxyimino group, imino wherein any of the C1-C5 carbons comprise part of said imino group, amidino wherein any of the C1-C5 carbons comprise part of said amidino group, sulfido, and sulfoxido,
(b) C3-C6 cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfido, and sulfoxido,
(c) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
   R₅ is a lone pair of electrons, hydrogen, or selected from the group consisting of:
(a) C1-C5 alkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C1-C5 carbons comprise part of said oxyimino group, imino wherein any of the C1-C5 carbons comprise part of said imino group, amidino wherein any of the C1-C5 carbons comprise part of said amidino group, sulfido, and sulfoxido,
(b) C3-C6 cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfido, and sulfoxido,
(c) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
   or R₄ and Y together form a ring of between 5 and 7 atoms where said ring is optionally fused or spiro in relation to the ring system of Y, said ring optionally being partially saturated or aromatic and optionally containing 1-2 additional heteroatoms selected from the group consisting of N, O, S, and a combination thereof;
   or R₄ and R₅ together form a ring of between 3 and 7 atoms where said ring is optionally substituted, said ring optionally being saturated, partially unsaturated or aromatic and optionally containing 1-2 additional heteroatoms selected from the group consisting of N, O, S, and a combination thereof;
   R₆ is hydrogen or an ester prodrug of the carboxylic acid;
   Z is optionally substituted: C1-C4 alkyl, C1-C4 alkoxy, C1-C4 sulfido, C3-C6 cycloalkyl, C3-C6 heterocyclyl where the bond to Y is through a carbon atom of said heterocyclyl ring, heteroaryl where the bond to Y is through a carbon atom of said heteraryl ring, oxyimino, imino, or amidino where the carbon of said oxyimino, imino, or amidino group is attached to Y;
   or Z and Y together form a ring of 5-7 atoms where said ring is optionally fused or spiro in relation to the ring system of Y, said ring optionally being partially saturated or aromatic and optionally containing 1-3 heteroatoms selected from the group consisting of N, O, S, and a combination thereof;
   or Z and R₄ together form a ring of 4-7 atoms where said ring optionally is saturated, partially unsaturated, or aromatic and optionally contains 1-2 additional heteroatoms selected from the group consisting of N, O, S, and a combination thereof;
   X₁ and X₂ are independently hydroxyl, halogen, NR₄R₅, C1-C6 alkoxy, or when taken together X₁ and X₂ form a cyclic boron ester where said chain or ring contains from 2 to 20 carbon atoms and, optionally, 1-3 heteroatoms selected from the group consisting of N, O, S and a combination thereof, or when taken together X₁ and X₂ form a cyclic boron amide where said chain or ring contains from 2 to 20 carbon atoms and, optionally, 1-3 heteroatoms selected from the group consisting of N, O, S, and a combination thereof, or when taken together X₁ and X₂ form a cyclic boron amide-ester where said chain contains from 2-20 carbon atoms and, optionally, 1-3 heteroatoms selected from the group consisting of N, O, S, and a combination thereof, or X₁ is hydroxyl and X₂ is replaced by the ortho-hydroxyl oxygen of the phenyl ring such that a 6-membered ring is formed;
   or a salt thereof;
   provided that when R₁, R₂, R₃, R₄, R₅, and R₆ are hydrogen, X₁ and X₂ are hydroxyl, n is 0, Y is phenyl, and Z is CH₂ then Z cannot be at the meta-position of the phenyl ring relative to the rest of the molecule.

Preferred embodiments are those compounds of Formula (II) wherein R₁ is hydrogen; R₂ and R₃ are independently hydrogen, or selected from the group consisting of hydroxyl, halogen, carboxyl, cyano, thiol, optionally substituted: C1-C5 alkyl, C1-C5 alkenyl, C1-C5 alkoxy, C3-C6 cycloalkyl, C3-C6 heterocyclyl, amino, sulfide, and sulfone;

n is 0 or 1;
Y is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(c) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
   R₄ is hydrogen, or selected from the group consisting of:
(a) C1-C5 alkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C1-C5 carbons comprise part of said oxyimino group, imino wherein any of the C1-C5 carbons comprise part of said imino group, amidino wherein any of the C1-C5 carbons comprise part of said amidino group, sulfido, and sulfoxido,
(b) C3-C6 cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfido, and sulfoxido,
(c) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
   R₅ is a lone pair of electrons, hydrogen, or selected from the group consisting of:
(a) C1-C5 alkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C1-C5 carbons comprise part of said oxyimino group, imino wherein any of the C1-C5 carbons comprise part of said imino group, amidino wherein any of the C1-C5 carbons comprise part of said amidino group, sulfido, and sulfoxido,
(b) C3-C6 cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfido, and sulfoxido,
(c) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
   or R₄ and Y together form a ring of between 5 and 7 atoms where said ring is optionally fused or spiro in relation to the ring system of Y, said ring optionally being partially saturated or aromatic and optionally containing 1-2 additional heteroatoms selected from the group consisting of N, O, S, and a combination thereof;
   or R₄ and R₅ together form a ring of between 3 and 7 atoms where said ring is optionally substituted and optionally is saturated, partially unsaturated or aromatic and optionally contains 1-2 additional heteroatoms selected from the group consisting of N, O, S, and a combination thereof;
   R₆ is hydrogen or an ester prodrug of the carboxylic acid;
   Z is optionally substituted: C1-C4 alkyl, C1-C4 alkoxy, C1-C4 sulfido, C3-C6 cycloalkyl, C3-C6 heterocyclyl where the bond to Y is through a carbon atom of said heterocyclyl ring, oxyimino, imino, or amidino where the carbon of said oxyimino, imino, or amidino group is attached to Y;
   or Z and Y together form a ring of 5-7 atoms where said ring is optionally fused or spiro in relation to the ring system of Y, said ring optionally being partially saturated or aromatic and optionally containing 1-3 heteroatoms selected from the group consisting of N, O, S, and a combination thereof;
   or Z and R₄ together form a ring of 4-7 atoms where said ring is optionally saturated, partially unsaturated, or aromatic and optionally contains 1-2 additional heteroatoms selected from the group consisting of N, O, S, and a combination thereof;
   X₁ and X₂ are hydroxyl, or when taken together X₁ and X₂ form a cyclic boron ester where said chain or ring contains from 2 to 20 carbon atoms and, optionally, 1-3 heteroatoms selected from the group consisting of N, O, S, and a combination thereof, or X₁ is hydroxyl and X₂ is replaced by the ortho-hydroxyl oxygen of the phenyl ring such that a 6-membered ring is formed;
   or a salt thereof;
   provided that when R₂, R₃, R₄, R₅, and R₆ are hydrogen, X₁ and X₂ are hydroxyl, n is 0, Y is phenyl, and Z is CH₂ then Z cannot be at the meta-position of the phenyl ring relative to the rest of the molecule.

Other preferred embodiments are those compounds of Formula (II) wherein R₁, R₂, R₃, R₄, and R₅ are hydrogen;

R₆ is hydrogen or an ester prodrug of the carboxylic acid;
n is 0 or 1;
Y is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(c) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
   Z is optionally substituted: C1-C4 alkyl, C1-C4 alkoxy, C1-C4 sulfido, C3-C6 cycloalkyl, C3-C6 heterocyclyl where the bond to Y is through a carbon atom of said heterocyclyl ring, oxyimino, imino, or amidino where the carbon of the oxyimino, imino, or amidino group is attached to Y;
   or Z and Y together form a ring of 5-7 atoms where said ring optionally is partially saturated or aromatic and optionally contains 1-2 additional heteroatoms selected from the group consisting of N, O, S, and a combination thereof;
   or Z and R₄ together form a ring of 4-7 atoms where said ring optionally is saturated, partially unsaturated or aromatic and optionally contains 1-2 additional heteroatoms selected from the group consisting of N, O, S, and a combination thereof;
   X₁ and X₂ are hydroxyl, or X₁ is hydroxyl and X₂ is replaced by the ortho-hydroxyl oxygen of the phenyl ring such that a 6-membered ring is formed; or a salt thereof;
   provided that when R₆ is hydrogen, X₁ and X₂ are hydroxyl, n is 0, Y is phenyl, and Z is CH₂ then Z cannot be at the meta-position of the phenyl ring relative to the rest of the molecule.

Certain other compounds of Formula (I) include compounds of Formula (III):

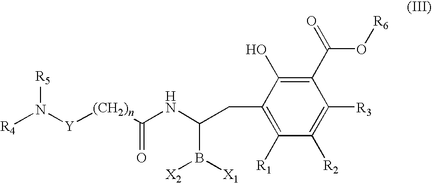

wherein R₁, R₂, and R₃ are independently hydrogen, or selected from the group consisting of hydroxyl, halogen, carboxyl, cyano, thiol, optionally substituted: C1-C5 alkyl, C1-C5 alkoxy, C1-C5 alkenyl, C3-C6 cycloalkyl, C3-C6 heterocyclyl, amino, sulfide, and sulfone;
n is 0, 1, or 2;
Y is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(c) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
   R₄ is hydrogen, or selected from the group consisting of:
(a) C1-C5 alkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C1-C5 carbons comprise part of said oxyimino group, imino wherein any of the C1-C5 carbons comprise part of said imino group, amidino wherein any of the C1-C5 carbons comprise part of said amidino group, sulfido, and sulfoxido,
(b) C3-C6 cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfido, and sulfoxido,
(c) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
   R₅ is a lone pair of electrons, hydrogen, or selected from the group consisting of:
(a) C1-C5 alkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C1-C5 carbons comprise part of said oxyimino group, imino wherein any of the C1-C5 carbons comprise part of said imino group, amidino wherein any of the C1-C5 carbons comprise part of said amidino group, sulfido, and sulfoxido,
(b) C3-C6 cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfido, and sulfoxido,
(c) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
   or R₄ and Y together form a ring of between 3 and 7 atoms where said ring is optionally fused or spiro in relation to the ring system of Y, said ring optionally being saturated, partially saturated or aromatic and optionally containing 1-2 additional heteroatoms selected from the group consisting of N, O, S, and a combination thereof;
   or R₄ and R₅ together form a ring of between 3 and 7 atoms where said ring is optionally substituted, said ring optionally being saturated, partially unsaturated or aromatic and optionally containing 1-2 additional heteroatoms selected from the group consisting of N, O, S, and a combination thereof;
   R₆ is hydrogen or an ester prodrug of the carboxylic acid;
   X₁ and X₂ are independently hydroxyl, halogen, NR₄R₅, C1-C6 alkoxy, or when taken together X₁ and X₂ form a cyclic boron ester where said chain or ring contains from 2 to 20 carbon atoms and, optionally, 1-3 heteroatoms selected from the group consisting of N, O, S and a combination thereof, or when taken together X₁ and X₂ form a cyclic boron amide where said chain or ring contains from 2 to 20 carbon atoms and, optionally, 1-3 heteroatoms selected from the group consisting of N, O, S, and a combination thereof, or when taken together X₁ and X₂ form a cyclic boron amide-ester where said chain contains from 2-20 carbon atoms and, optionally, 1-3 heteroatoms selected from the group consisting of N, O, S, and a combination thereof, or X₁ is hydroxyl and X₂ is replaced by the ortho-hydroxyl oxygen of the phenyl ring such that a 6-membered ring is formed;
   or a salt thereof;
   provided that when R₁, R₂, R₃, R₄, and R₆ are hydrogen, R₅ is hydrogen or CH₃C(O)—, X₁ and X₂ are hydroxyl, n is 1, Y is 4-thiazolyl, then NR₄R₅ cannot be located at the 2-position of the thiazole ring;
   further provided that when R₁, R₂, R₃, and R₆ are hydrogen, n is 0, Y is phenyl, and NR₄R₅ is 1-imidazolyl, then NR₄R₅ cannot be located at the 3-position of the phenyl ring relative to the rest of the molecule;
   further provided that when R₁, R₂, R₃, and R₆ are hydrogen, n is 0, Y is 5-pyridyl, and NR₄R₅ is 4-morpholinyl, then NR₄R₅ cannot be located at the 2-position of the pyridyl ring.

Preferred embodiments are those compounds of Formula (III) wherein R₁ is hydrogen; R₂ and R₃ are independently hydrogen, or selected from the group consisting of hydroxyl, halogen, carboxyl, cyano, thiol, optionally substituted: C1-C5 alkyl, C1-C5 alkenyl, C1-C5 alkoxy, C3-C6 cycloalkyl, C3-C6 heterocyclyl, amino, sulfide, and sulfone;

n is 0, 1, or 2;
Y is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(c) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
   R₄ is hydrogen, or selected from the group consisting of:
(a) C1-C5 alkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C1-C5 carbons comprise part of said oxyimino group, imino wherein any of the C1-C5 carbons comprise part of said imino group, amidino wherein any of the C1-C5 carbons comprise part of said amidino group, sulfido, and sulfoxido,
(b) C3-C6 cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfido, and sulfoxido,
(c) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
   R₅ is a lone pair of electrons, hydrogen, or selected from the group consisting of:
(a) C1-C5 alkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C1-C5 carbons comprise part of said oxyimino group, imino wherein any of the C1-C5 carbons comprise part of said imino group, amidino wherein any of the C1-C5 carbons comprise part of said amidino group, sulfido, and sulfoxido,
(b) C3-C6 cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfido, and sulfoxido,
(c) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
   or R₄ and Y together form a ring of between 3 and 7 atoms where said ring is optionally fused or spiro in relation to the ring system of Y, said ring optionally being saturated, partially saturated or aromatic and optionally containing 1-2 additional heteroatoms selected from the group consisting of N, O, S, and a combination thereof;
   or R₄ and R₅ together form a ring of between 3 and 7 atoms where said ring is optionally substituted and optionally is saturated, partially unsaturated or aromatic and optionally contains 1-2 additional heteroatoms selected from the group consisting of N, O, S, and a combination thereof;
   R₆ is hydrogen or an ester prodrug of the carboxylic acid;
   X₁ and X₂ are hydroxyl, or when taken together X₁ and X₂ form a cyclic boron ester where said chain or ring contains from 2 to 20 carbon atoms and, optionally, 1-3 heteroatoms selected from the group consisting of N, O, S, and a combination thereof, or X₁ is hydroxyl and X₂ is replaced by the ortho-hydroxyl oxygen of the phenyl ring such that a 6-membered ring is formed;
   or a salt thereof;
   provided that when R₂, R₃, R₄, and R₆ are hydrogen, R₅ is hydrogen or CH₃C(O)—, X₁ and X₂ are hydroxyl, n is 1, Y is 4-thiazolyl, then NR₄R₅ cannot be located at the 2-position of the thiazole ring;
   further provided that when R₂, R₃, and R₆ are hydrogen, n is 0, Y is phenyl, and NR₄R₅ is 1-imidazolyl, then NR₄R₅ cannot be located at the 3-position of the phenyl ring relative to the rest of the molecule;
   further provided that when R₂, R₃, and R₆ are hydrogen, n is 0, Y is 5-pyridyl, and NR₄R₅ is 4-morpholinyl, then NR₄R₅ cannot be located at the 2-position of the pyridyl ring.

Other preferred embodiments are those compounds of Formula (III) wherein R₁, R₂, R₃, R₄, and R₅ are hydrogen;

R₆ is hydrogen or an ester prodrug of the carboxylic acid;
n is 0 or 1;
Y is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(c) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
   X₁ and X₂ are hydroxyl, or X₁ is hydroxyl and X₂ is replaced by the ortho-hydroxyl oxygen of the phenyl ring such that a 6-membered ring is formed; or a salt thereof;
   provided that when R₆ is hydrogen, X₁ and X₂ are hydroxyl, n is 1, Y is 4-thiazolyl, then NR₄R₅ cannot be located at the 2-position of the thiazole ring; further provided that when R₆ is hydrogen, n is 0, Y is phenyl, and NR₄R₅ is 1-imidazolyl, then NR₄R₅ cannot be located at the 3-position of the phenyl ring relative to the rest of the molecule;
   further provided that when R₆ is hydrogen, n is 0, Y is 5-pyridyl, and NR₄R₅ is 4-morpholinyl, then NR₄R₅ cannot be located at the 2-position of the pyridyl ring.

Add additional fall back positions around LIH111 and LIQ849

Another aspect is for the compound of Formula (I) to be a compound selected from the group consisting of:

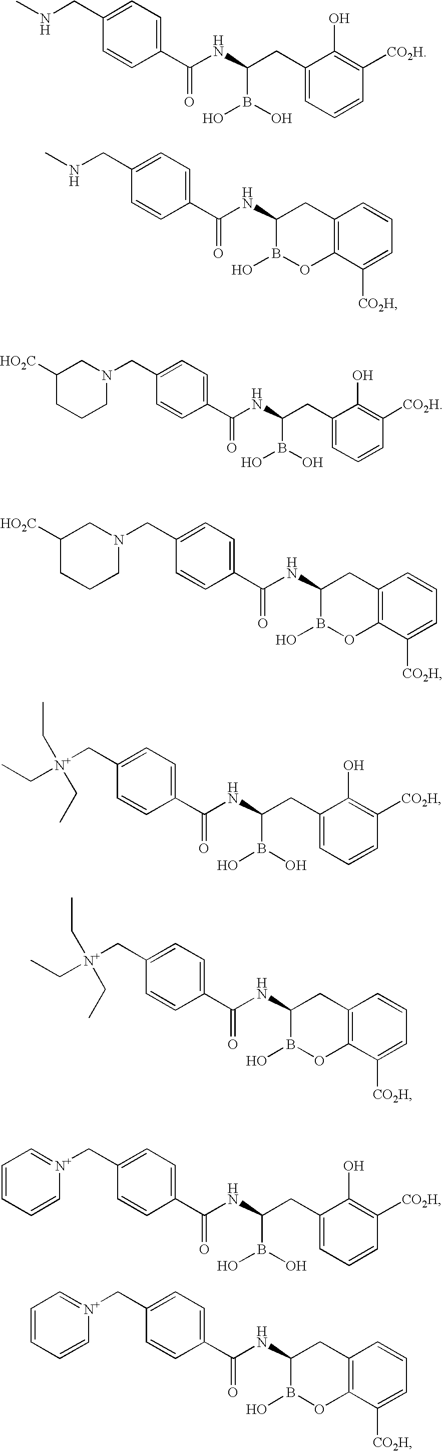

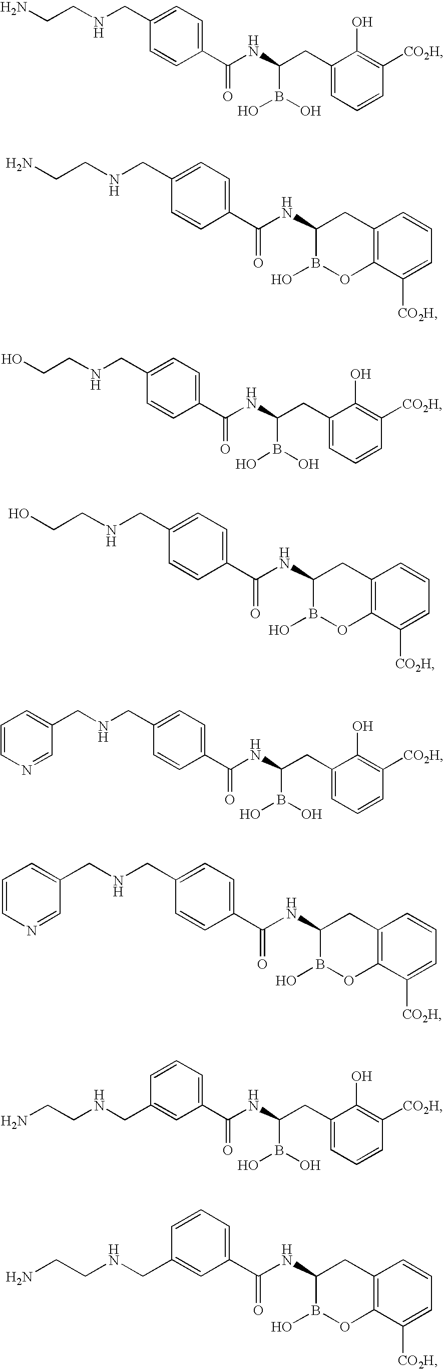

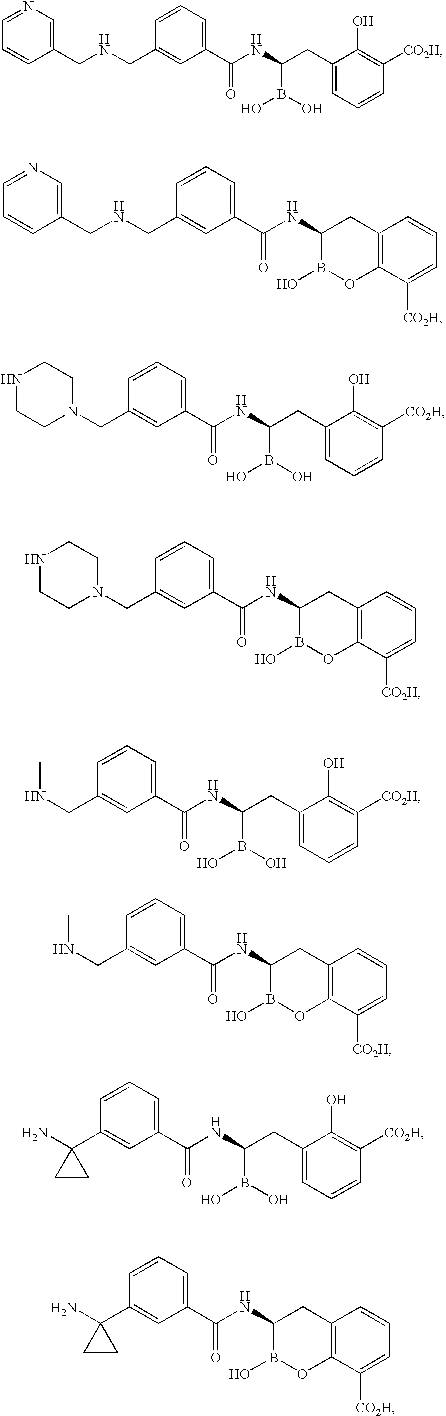

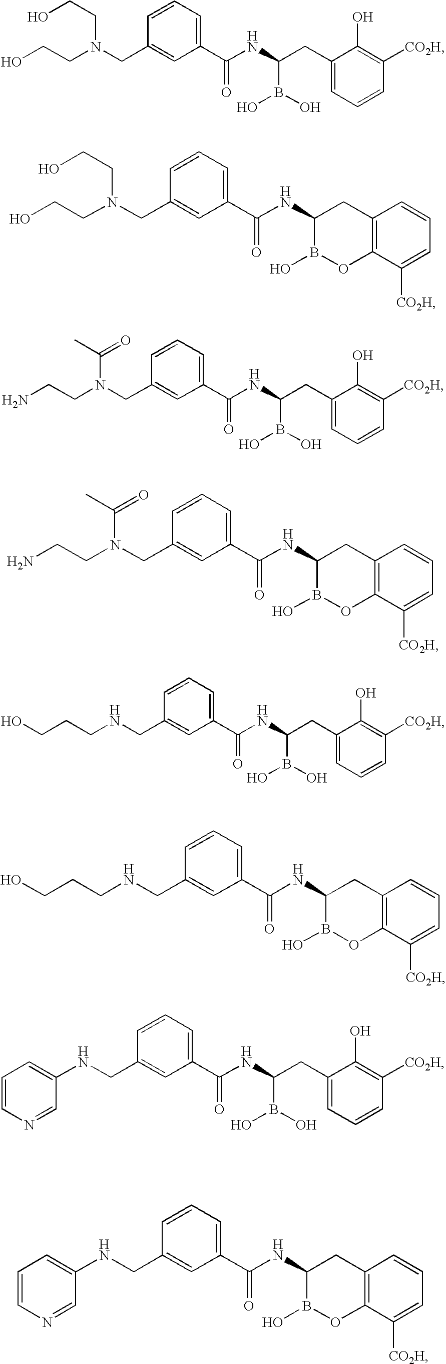

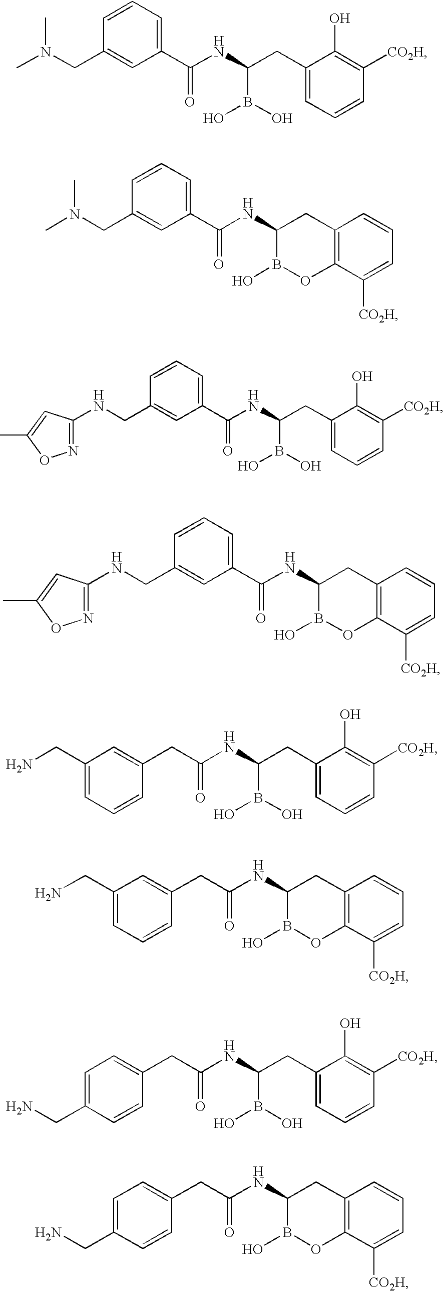

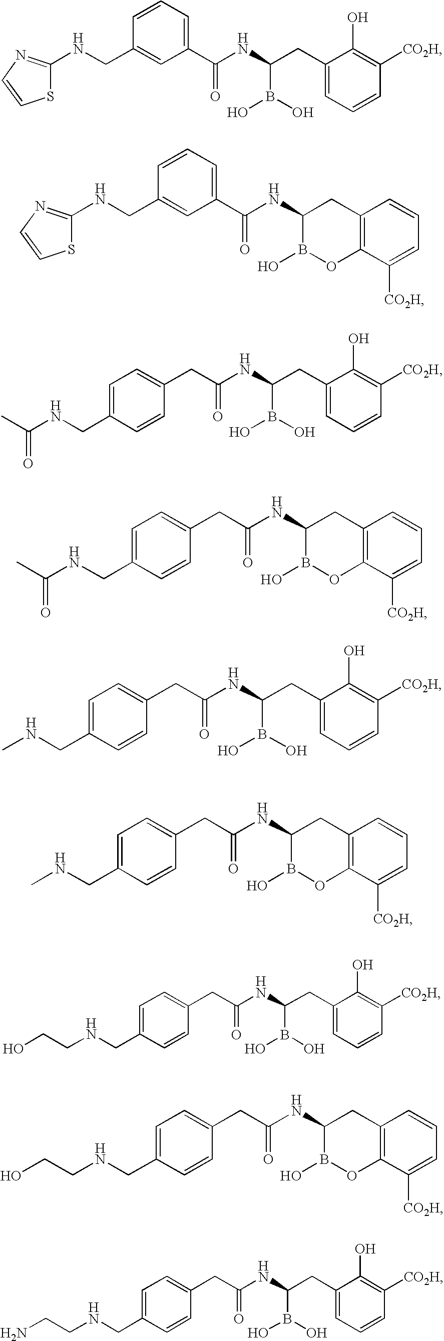

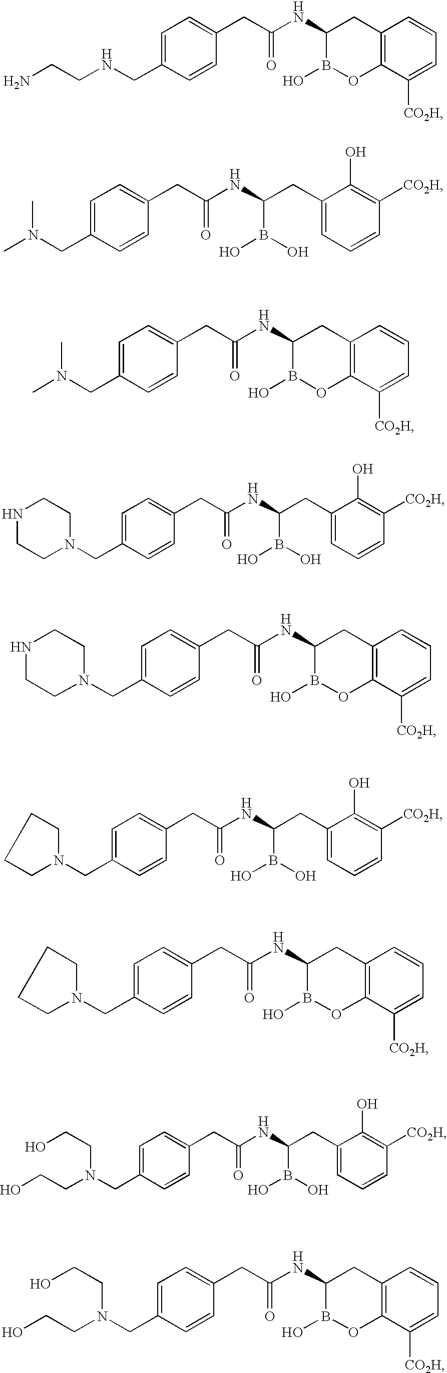

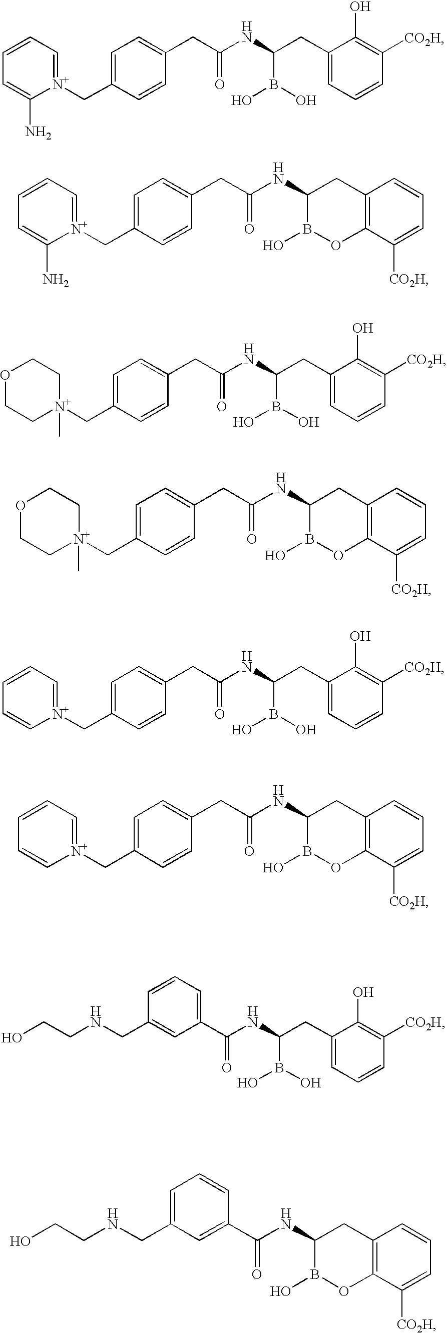

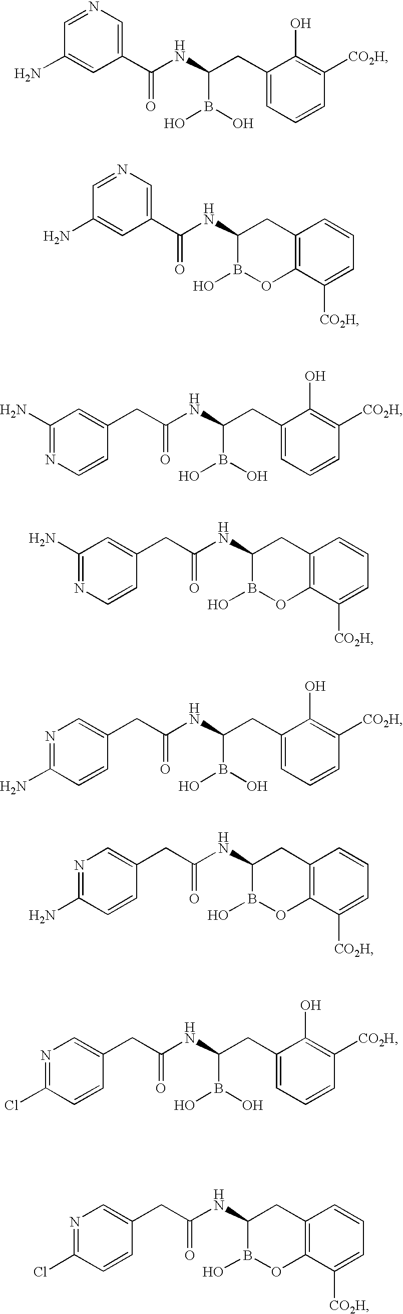

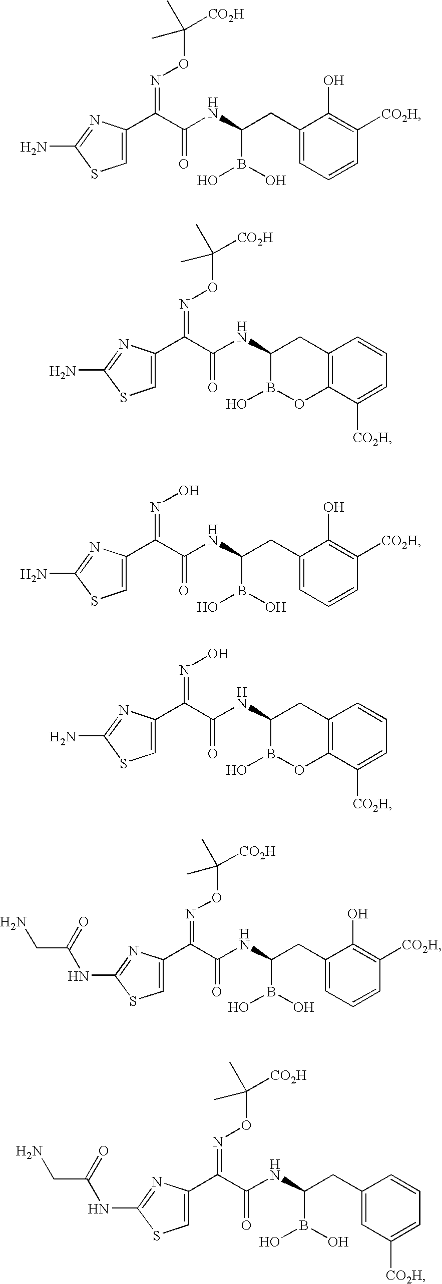

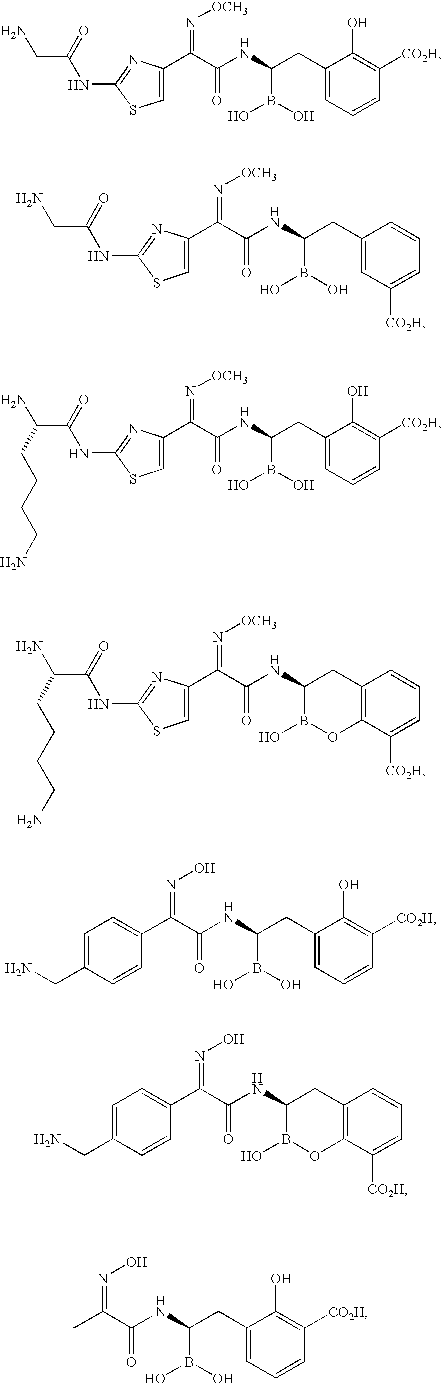

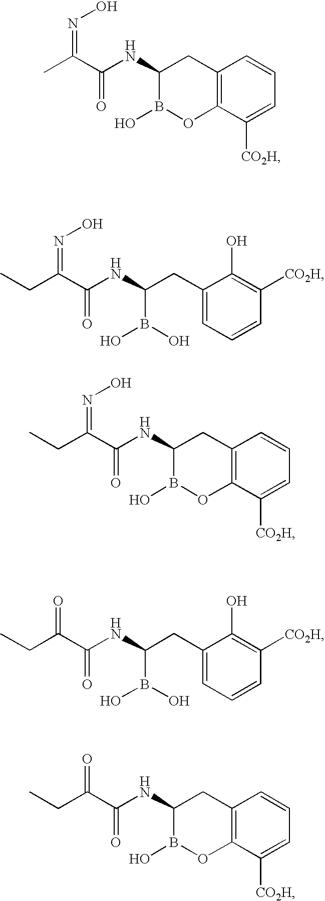

and a salt thereof. In some embodiments, the compound is selected from the group consisting of

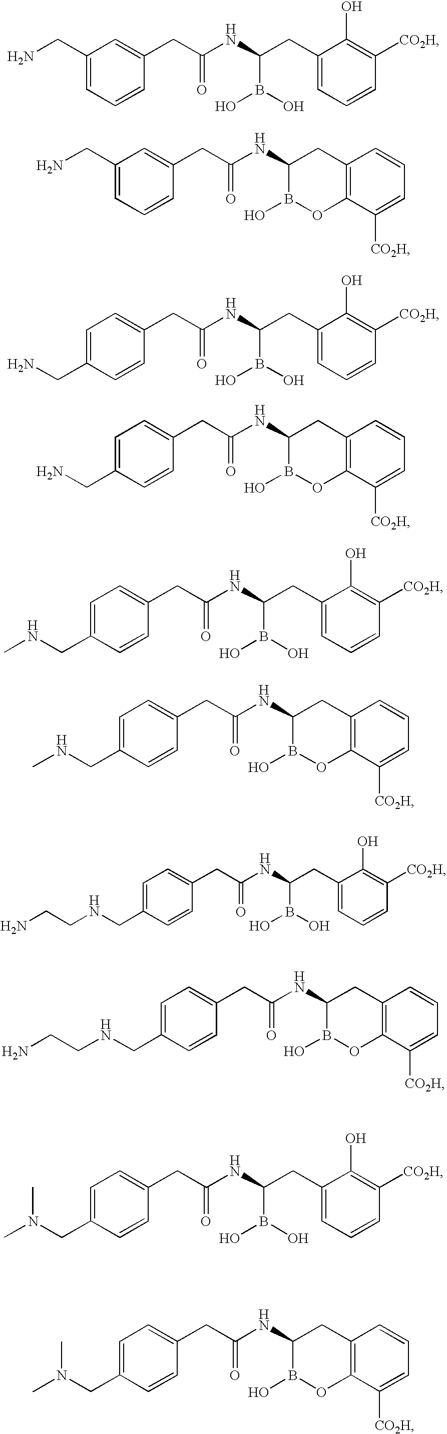

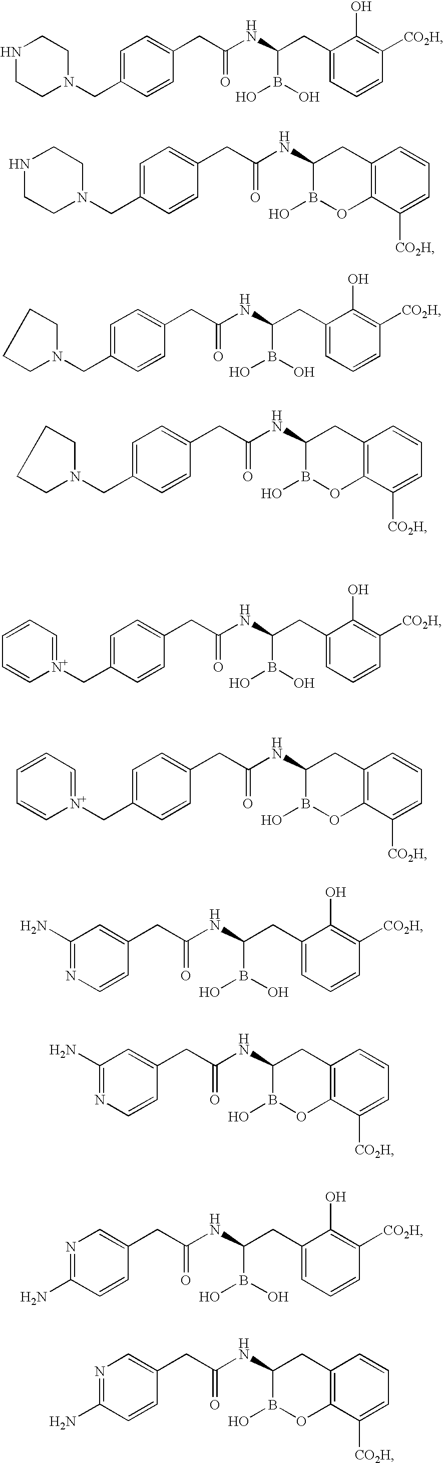

and a salt thereof.

Another aspect is for a pharmaceutical composition comprising: (a) one or more compounds discussed above; (b) one or more β-lactam antibiotics; and (c) one or more pharmaceutically acceptable carriers.

A further aspect is for a pharmaceutical composition comprising: (a) one or more compounds discussed above; and (b) one or more pharmaceutically acceptable carriers.

An additional aspect is for a method of treating a bacterial infection in a mammal comprising administering to a mammal in need thereof (a) one or more of the compounds discussed above and (b) an effective amount of a β-lactam antibiotic.

Another aspect is for a method of treating a bacterial infection in a mammal comprising administering to a mammal in need thereof an effective amount of a compound discussed above.

An additional aspect is for use of one or more compounds discussed above in the manufacture of a medicament for the treatment of a bacterial infection.

As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers or axes and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the terms “salt” or “salts” refers to an acid addition or base addition salt of a compound of the invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulformate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. The invention includes various isotopically labeled compounds as defined herein, for example those into which radioactive isotopes, such as ³H, ¹³C, and ¹⁴C, are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Compounds of the invention, i.e. compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviating, inhibiting, preventing and/or ameliorating a condition, or a disorder or a disease (i) mediated by one or more beta lactamase(s), or (ii) associated with beta lactamase activity; or (2) reducing or inhibiting the activity of one or more beta lactamase(s). In another non-limiting embodiment, the term “a therapeutically effective amount” refers to the amount of the compound of the present invention that, when administered to a cell, or a tissue, or a non-cellular biological material, or a medium, is effective to at least partially reducing or inhibiting the activity of at least one beta lactamase; or at least partially reducing or inhibiting the expression of at least one beta lactamase.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated bonds may, if possible, be present in cis-(Z)- or trans-(E)-form.
Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Beta-Lactamase Inhibitor Synthesis

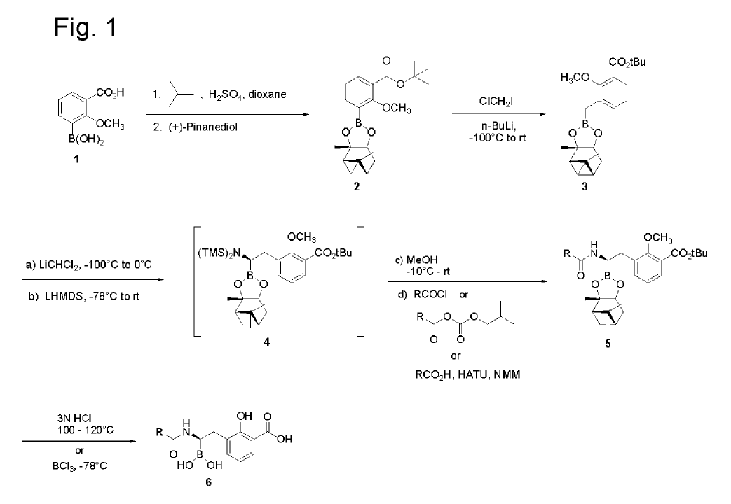

The desired boronic acid containing compounds can be prepared from commercially available arylboronic acids using the general synthetic route depicted in FIG. 1. The carboxylic acid group of 1 is first protected as the tert-butyl ester using 2-methylpropene in the presence of catalytic sulfuric acid, and the boronic acid is then subsequently converted to the chiral boronic ester 2 with (+)-pinanediol. Homologation using (chloromethyl)lithium as described by Sadhu and Matteson, Organometallics, 1985, 4, 1687-1689 affords the benzylboronic ester 3. Conversion to the bis(trimethylsilyl)amine intermediate 4 can be achieved using the conditions described by Schoichet et al., J. Am. Chem. Soc. 2003, 125, 685-695. This could then be converted to the desired amides 5 by reaction with an acid chloride or other active ester such as that derived from the reaction of a carboxylic acid with isobutyl chloroformate or from the reaction of a carboxylic acid with a tetramethyluronium agent such as O-(7-Azabenzotriazol-1-yl)-N,N,N′,N′-tetramethyluronium hexafluorophosphate (HATU). Removal of the pinanediol group and deprotection of the carboxylic acid, the phenol, and any other acid labile protecting group can be accomplished in one step under acidic conditions such as aqueous HCl in dioxane or BCl₃ or BBr₃ in dichloromethane.

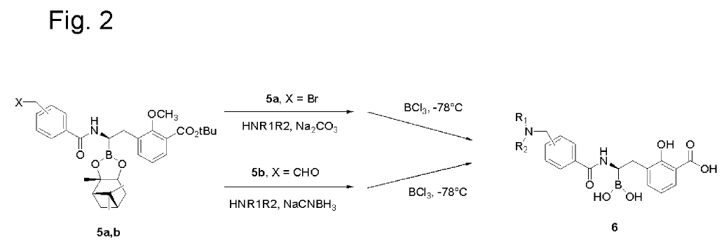

Compounds of general structure 6 wherein the R group is an aminomethylbenzamide can be synthesized as depicted in FIG. 2. Compound 5a, where X is bromine, can be treated with an amine HNR₁R₂ in the presence of an inorganic base such as Na₂CO₃ to afford the intermediate benzylamine. Treatment with BCl₃ provides the fully deprotected boronic acid 6. Alternatively, reaction of the carboxaldehyde of 5b with an amine in the presence of a reducing agent such as NaCNBH₃ will result in the formation of an intermediate benzylamine which can be converted to the final boronic acid 6 with BCl₃.

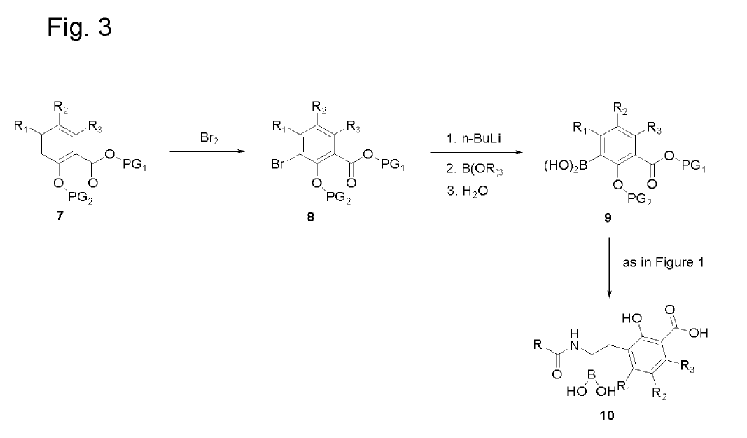

Alternatively, the arylboronic acid can be prepared from the corresponding bromosalicylic acid as shown in FIG. 3 where PG1 and PG2 are protecting groups that may or may not be the same. Substituted salicyclic acids are known in the literature, and one skilled in the art will recognize that there are numerous ways to append a boronic acid or ester group to obtain the desired intermediates.

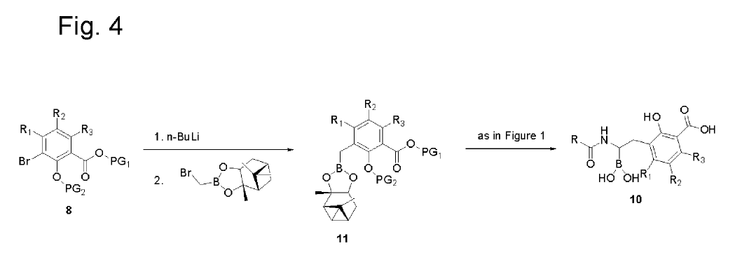

For example, electrophilic aromatic bromination of a salicylic acid derivative 7 can provide the desired 3-bromosalicylic acid 8 (cf. Wang et al, Bioorg. Med. Chem. Letters, 2007, 17(10), 2817-2822). Conversion of the arylbromide to an organometallic species, for example by the action of n-butyllithium at temperatures at or below −78° C., followed by reaction with a trialkylborate, for example trimethylborate, and subsequent hydrolysis then gives the arylboronic acid 9. Conversion to the final product is then accomplished following the synthetic sequence shown in FIG. 1. The benzylboronic ester 10 can also be prepared from the same organometallic intermediate by reaction with alpha-halomethylboronic esters, for example (+)-pinanediol-(bromomethyl)boronate as shown in FIG. 4 (cf. Matteson et al, Organometallics, 1996, 15(1), 152). The benzylboronic ester 10 can then be carried through the sequence shown in FIG. 1.

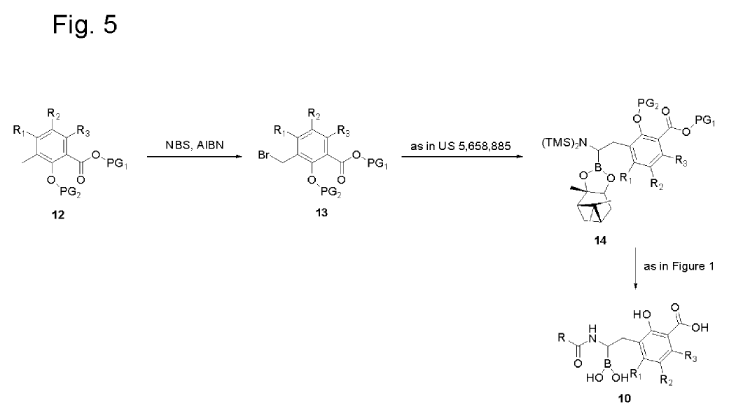

Alternatively, the desired compounds can be obtained from appropriately protected 3-methylsalicyclic acids as shown in FIG. 5. Bromination of the methyl group of 12, for example with N-bromosuccinimide (NBS) in the presence of a free radical initiator such as 2,2′-Azobis(2-methylpropionitrile) (AIBN) affords the benzylic bromide 13. Conversion to the bis(trimethylsilyl)amine intermediate 14 can be performed via the benzylic anion, for example as described in U.S. Pat. No. 5,658,885, and then conversion to the desired compounds can be accomplished as shown in FIG. 1.

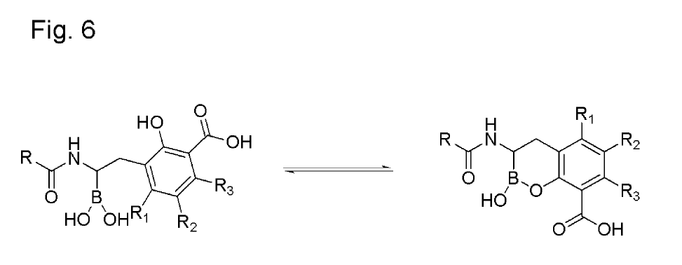

Based on literature precedent, it is assumed that Applicants obtain predominantly the 1-(R) enantiomer when using (+)-pinanediol to form the boronic ester, although one skilled in the art will recognize that minor amounts of the 1-(S) isomer may be present in the reaction products. Also, there is a possibility that these compounds can exist either as the free boronic acid or as the cyclic boronate ester, or as a mixture of the cyclic form and the open chain form as depicted in FIG. 6 (Strynadka et al. Biochemistry, 2000, 39(18), 5312-5321).

### Prodrug Synthesis

In order to minimize toxicity problems, or to optimize delivery prospects, therapeutic agents can sometimes be advantageously presented to patients in the form of prodrugs. Prodrugs are molecules capable of being converted to drugs (active therapeutic compounds) in vivo by certain chemical or enzymatic modifications of their structure. Prodrugs are designed to overcome pharmaceutically and/or pharmacokinetically based problems associated with the parent drug molecule that would otherwise limit the clinical usefulness of the drug. The advantage of a prodrug lies in its physical properties, such as enhanced water solubility for parenteral administration at physiological pH compared to the parent drug, or enhanced absorption from the digestive tract after oral administration, or enhanced drug stability for long-term storage.

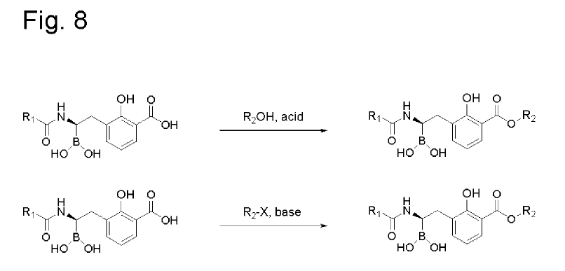

FIG. 8 illustrates two general methods for the synthesis of ester prodrugs of the beta-lactamase inhibitors. Heating a solution of the carboxylic acid, prepared as shown in FIG. 1, with an alcohol R₂OH in the presence of an acid such as hydrochloric or sulfuric acid will afford the desired ester prodrug. Alternatively, the carboxylic acid can be esterified using an alkyating agent R₂—X, where X represents a leaving group such as Br, I or OSO₂R, in the presence of a base such as NaHCO₃, Cs₂CO₃ or NaOH.

### Administration of Beta-Lactamase Inhibitors

Beta-lactamase inhibitors can be administered to subjects in a biologically compatible form suitable for pharmaceutical administration in vivo to, e.g., increase antibacterial activity of beta-lactam antibiotics. Administration of a beta-lactamase inhibitor as described herein can be in any pharmacological form including a therapeutically active amount of a beta-lactamase inhibitor alone or in combination with a pharmaceutically acceptable carrier.

A therapeutically active amount of a beta-lactamase inhibitor may vary according to factors such as the disease state, age, sex, and weight of the subject, and the ability of the beta-lactamase inhibitor to elicit a desired response in the subject. Dosage regimes may be adjusted to provide the optimum therapeutic response. For example, several divided doses may be administered daily, or the dose may be proportionally reduced as indicated by the exigencies of the therapeutic situation.

The therapeutic or pharmaceutical compositions can be administered by any suitable route known in the art including, for example, intravenous, subcutaneous, intramuscular, transdermal, intrathecal, or intracerebral or administration to cells in ex vivo treatment protocols. Administration can be either rapid as by injection or over a period of time as by slow infusion or administration of slow release formulation.

A beta-lactamase inhibitor can also be linked or conjugated with agents that provide desirable pharmaceutical or pharmacodynamic properties. For example, a beta-lactamase inhibitor can be coupled to any substance known in the art to promote penetration or transport across the blood-brain barrier such as an antibody to the transferrin receptor, and administered by intravenous injection (see, e.g., Friden P M et al., Science 259:373-77 (1993)). Furthermore, a beta-lactamase inhibitor can be stably linked to a polymer such as polyethylene glycol to obtain desirable properties of solubility, stability, half-life, and other pharmaceutically advantageous properties (see, e.g., Davis at al., Enzyme Eng. 4:169-73 (1978); Burnham N L, Am. J. Hosp. Pharm. 51:210-18 (1994)).

Furthermore, a beta-lactamase inhibitor can be in a composition which aids in delivery into the cytosol of a cell. For example, the beta-lactamase inhibitor may be conjugated with a carrier moiety such as a liposome that is capable of delivering the beta-lactamase inhibitor into the cytosol of a cell. Such methods are well known in the art (see, e.g., Amselem S et al., Chem. Phys. Lipids 64:219-37 (1993)). Alternatively, a beta-lactamase inhibitor can be modified to include specific transit peptides or fused to such transit peptides which are capable of delivering their beta-lactamase inhibitor into a cell. In addition, the beta-lactamase inhibitor can be delivered directly into a cell by microinjection.

The compositions are usually employed in the form of pharmaceutical preparations. Such preparations are made in a manner well known in the pharmaceutical art. One preferred preparation utilizes a vehicle of physiological saline solution, but it is contemplated that other pharmaceutically acceptable carriers such as physiological concentrations of other non-toxic salts, five percent aqueous glucose solution, sterile water, or the like may also be used. As used herein, “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any standard media or agent is incompatible with the active compound, use thereof in the therapeutic compositions is contemplated. Supplementary active compounds can also be incorporated into the compositions. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. A beta-lactamase inhibitor can also be incorporated into a solid or semi-solid biologically compatible matrix which can be implanted into tissues.

The carrier can contain other pharmaceutically-acceptable excipients for modifying or maintaining the pH, osmolarity, viscosity, clarity, color, sterility, stability, rate of dissolution, or odor of the formulation. Such excipients are those substances usually and customarily employed to formulate dosages for parenteral administration in either unit dosage or multi-dose form or for direct infusion by continuous or periodic infusion.

In some embodiments, the pharmaceutical compositions further comprise an effective amount of a beta-lactam antibiotic. Exemplary β-lactam antibiotics include penicillins, cephalosporins, carbapenems, monobactams, bridged monobactams, or a combination thereof. Penicillins include, but are not limited to, benzathine penicillin, benzylpenicillin, phenoxymethylpenicillin, procaine penicillin, oxacillin, methicillin, dicloxacillin, flucloxacillin, temocillin, amoxicillin, ampicillin, co-amoxiclav, azlocillin, carbenicillin, ticarcillin, mezlocillin, piperacillin, apalcillin, hetacillin, bacampicillin, sulbenicillin, mecicilam, pevmecillinam, ciclacillin, talapicillin, aspoxicillin, cloxacillin, nafcillin, pivampicillin, or a combination thereof. Cephalosporins include, but are not limited to, cephalothin, cephaloridin, cefaclor, cefadroxil, cefamandole, cefazolin, cephalexin, cephradine, ceftizoxime, cefoxitin, cephacetril, cefotiam, cefotaxime, cefsulodin, cefoperazone, ceftizoxime, cefinenoxime, cefinetazole, cephaloglycin, cefonicid, cefodizime, cefpirome, ceftazidime, ceftriaxone, cefpiramide, cefbuperazone, cefozopran, cefepim, cefoselis, cefluprenam, cefuzonam, cefpimizole, cefclidin, cefixime, ceftibuten, cefdinir, cefpodoxime axetil, cefpodoxime proxetil, cefteram pivoxil, cefetamet pivoxil, cefcapene pivoxil, cefditoren pivoxil, cefuroxime, cefuroxime axetil, loracarbacef, latamoxef, anti-methicillin-resistant Staphylococcus aureus (MRSA) cephalosporins (e.g., ceftobiprole or ceftaroline), FR264205 (see Takeda et al., Antimicrob. Agents Chemother. 51:826-30 (2007)), or a combination thereof. Carbapenems include, but are not limited to, imipenem, meropenem, ertapenem, faropenem, doripenem, biapenem, panipenem, anti-MRSA carbapenems (e.g., PZ-601 or ME1036, see Expert Rev. Anti-Infect. Ther. (2008) 6:39-49), or a combination thereof. Monobactams include, but are not limited to, aztreonam, carumonam, BAL30072 (Basilea Poster F1-1173, Ann. Interscience Conf. Antimicrob. Agents Chemother. (2008)), or a combination thereof. See FIG. 6 for structures of PZ-601, ME1036, and BAL30072.

The beta-lactamase inhibitors or their pharmaceutically acceptable salts may be administered at the same time as the dose of beta-lactam antibiotics or separately. This may be carried out in the form of a mixture of the two active ingredients or in the form of a pharmaceutical combination of the two separate active ingredients.

The dosage of the beta-lactamase inhibitors and of their pharmaceutically acceptable salts may vary within wide limits and should naturally be adjusted, in each particular case, to the individual conditions and to the pathogenic agent to be controlled. In general, for a use in the treatment of bacterial infections, the daily dose may be between 0.250 g and 10 g per day, by the oral route in humans, or else between 0.25 g and 10 g per day by the intramuscular or intravenous route. Moreover, the ratio of the beta-lactamase inhibitor or of the pharmaceutically acceptable salt thereof to the beta-lactam antibiotic may also vary within wide limits and should be adjusted, in each particular case, to the individual conditions. In general, a ratio ranging from about 1:20 to about 1:1 is recommended.

Dose administration can be repeated depending upon the pharmacokinetic parameters of the dosage formulation and the route of administration used.

It is also provided that certain formulations containing a beta-lactamase inhibitor are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents, or flavoring agents. The compositions may be formulated so as to provide rapid, sustained, or delayed release of the active ingredients after administration to the patient by employing procedures well known in the art. The formulations can also contain substances that diminish proteolytic degradation and/or substances which promote absorption such as, for example, surface active agents.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved and (b) the limitations inherent in the art of compounding such an active compound for the treatment of sensitivity in individuals. The specific dose can be readily calculated by one of ordinary skill in the art, e.g., according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated; the choice of composition to be administered; the age, weight, and response of the individual patient; the severity of the patient's symptoms; and the chosen route of administration.

Toxicity and therapeutic efficacy of such compounds can be determined by standard pharmaceutical procedures in cell cultures or experimental animals, for example, for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the methods disclosed herein, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Inhibition of Bacterial Growth

The present disclosure also provides methods for inhibiting bacterial growth, by e.g. reducing bacterial resistance to a β-lactam antibiotic, such methods comprising contacting a bacterial cell culture, or a bacterially infected cell culture, tissue, or organism, with a beta-lactamase inhibitor described herein. Preferably, the bacteria to be inhibited by administration of a beta-lactamase inhibitor of the invention are bacteria that are resistant to beta-lactam antibiotics. More preferably, the bacteria to be inhibited are beta-lactamase positive strains that are highly resistant to beta-lactam antibiotics. The terms “resistant” and “highly resistant” are well-understood by those of ordinary skill in the art (see, e.g., Payne et al., Antimicrobial Agents and Chemotherapy 38:767-772 (1994); Hanaki et al., Antimicrobial Agents and Chemotherapy 30:1120-1126 (1995)). Preferably, highly resistant bacterial strains are those against which the MIC of methicillin is >100 μg/mL. Preferably, slightly resistant bacterial strains are those against which the MIC of methicillin is >25 μg/mL.

These methods are useful for inhibiting bacterial growth in a variety of contexts. In certain preferred embodiments, the compound of the invention is administered to an experimental cell culture in vitro to prevent the growth of beta-lactam resistant bacteria. In certain other preferred embodiments the compound of the invention is administered to a mammal, including a human, to prevent the growth of beta-lactam resistant bacteria in vivo. The method according to this embodiment of the invention comprises administering a therapeutically effective amount of a beta-lactamase inhibitor for a therapeutically effective period of time to a mammal, including a human. Preferably, the beta-lactamase inhibitor is administered in the form of a pharmaceutical composition as described supra. In some embodiments, a beta-lactam antibiotic is co-administered with the beta-lactamase inhibitor as described supra.

Assays for the inhibition of beta-lactamase activity are well known in the art. For instance, the ability of a compound to inhibit beta-lactamase activity in a standard enzyme inhibition assay may be used (see, e.g., Page, Biochem J. 295:295-304 (1993)). Beta-lactamases for use in such assays may be purified from bacterial sources or, preferably, are produced by recombinant DNA techniques, since genes and cDNA clones coding for many beta-lactamases are known (see, e.g., Cartwright & Waley, Biochem J. 221:505-12 (1984)). Alternatively, the sensitivity of bacteria known, or engineered, to produce a beta-lactamase to an inhibitor may be determined. Other bacterial inhibition assays include agar disk diffusion and agar dilution (see, e.g., Traub & Leonhard, Chemotherapy 43:159-67 (1997)). Thus, a beta-lactamase can be inhibited by contacting the beta-lactamase enzyme with an effective amount of an inventive compound or by contacting bacteria that produce the beta-lactamase enzymes with an effective amount of such a compound so that the beta-lactamase in the bacteria is contacted with the inhibitor. The contacting may take place in vitro or in vivo. “Contacting” means that the beta-lactamase and the inhibitor are brought together so that the inhibitor can bind to the beta-lactamase. Amounts of a compound effective to inhibit a beta-lactamase may be determined empirically, and making such determinations is within the skill in the art. Inhibition includes both reduction and elimination of beta-lactamase activity.

### EXAMPLES

The disclosure herein is further defined in the following Examples. It should be understood that these Examples, while indicating preferred embodiments, are given by way of illustration only. From the above discussion and these Examples, one skilled in the art can ascertain the preferred features, and without departing from the spirit and scope thereof, can make various changes and modifications to adapt it to various uses and conditions.

### Example 1

### 2(R)-3-[2-(3-(Aminomethyl)benzoylamino)-2-borono-ethyl]-2-hydroxy-benzoic acid hydrochloride

Step 1. Synthesis of 3-Borono-2-methoxybenzoic acid tert-butyl ester. To a solution of 3-borono-2-methoxybenzoic acid (Combi-blocks, 5.0 g, 25.5 mmole) in 1,4-dioxane (30 mL) in a sealed tube was added conc. H₂SO₄ (1.5 mL). The solution was cooled to 0° C., and an equal volume of 2-methylpropene was bubbled in. The tube was sealed and allowed to stir at ambient temperature for 18 h. The solution was cooled in an ice bath, the seal was opened and the solution stirred at ambient temperature for 30 min. The solution was basified with saturated aq. NaHCO₃ and extracted twice with ethyl acetate (EtOAc). The combined organic layers were washed with water (5×), brine, dried (Na₂SO₄) and concentrated in vacuo to afford 4.0 g (62%) of the product as a white solid. ESI-MS m/z 275 (M+Na)⁺.

Step 2. Synthesis of 2-Methoxy-3-(2,9,9-trimethyl-3,5-dioxa-4-bora-tricyclo[6.1.1.0^{2,6}]dec-4-yl)-benzoic acid tert-butyl ester. A solution of 3-borono-2-methoxybenzoic acid tert-butyl ester (4.0 g, 15.9 mmole), tetrahydrofuran (THF, 21 mL), and (+)-pinanediol (2.70 g, 15.9 mmole) was stirred at room temperature for 1 h. The solution was concentrated in vacuo, and the residue chromatographed on SiO₂ with 6% EtOAc/hexane to afford 5.0 g (86%) of the product as a slowly crystallizing solid. ESI-MS m/z 409 (M+Na)⁺.

Step 3. Synthesis of 2-Methoxy-3-(2,9,9-trimethyl-3,5-dioxa-4-bora-tricyclo[6.1.1.0^{2,6}]dec-4-ylmethyl)-benzoic acid tert-butyl ester. A solution of 2-methoxy-3-(2,9,9-trimethyl-3,5-dioxa-4-bora-tricyclo[6.1.1.0^{2,6}]dec-4-yl)-benzoic acid tert-butyl ester (8.5 g, 22 mmol) and chloroiodomethane (4.6 g, 26.4 mmol) in THF (65 mL) under argon was cooled to −100° C. [MeOH, liq. N₂ slush bath]. n-BuLi (10.56 mL, 2.5M in hexane, 26.4 mmol) was added dropwise over a period of 10 minutes and the mixture stirred overnight. The reaction was quenched with H₂O (100 mL) and the aqueous phase was extracted with EtOAc (3×75 mL), the combined organic layers were dried over MgSO₄, and concentrated in vacuo. Purification by flash column chromatography on silica gel [R_{f}=0.21, (dichloromethane (DCM)/Hexane, 70:30, v/v)] afforded 8 g of the resultant compound as a colorless oil in 91% yield. ESI-MS m/z 401 (MH)⁺.

Step 4. Synthesis of 3-[2-[3-(tert-Butoxycarbonylamino-methyl)-benzoylamino]-2-(2,9,9-trimethyl-3,5-dioxa-4-bora-tricyclo6.1.1.0^{2,6}]dec-4-yl)-ethyl]-2-methoxy-benzoic acid tert-butyl ester. To anhydrous CH₂Cl₂ (1.4 mL, 21.8 mmol) in anhydrous THF (53 mL) under argon at −100° C. [MeOH, liq. N₂ slush bath], n-BuLi (8.1 mL, 2.5M in hexane, 22.2 mmol) was added dropwise and the mixture was stirred for 30 minutes. A THF (12 mL) solution of 2-Methoxy-3-(2,9,9-trimethyl-3,5-dioxa-4-bora-tricyclo[6.1.1.0^{2,6}]dec-4-ylmethyl)-benzoic acid tert-butyl ester (6.73 g, 16.81 mmol) was added over a period of 20 minutes. After 40 minutes the cooling bath was removed and the mixture warmed slowly to 0° C. After 2 hours the reaction flask was cooled to −78° C., LHMDS (18.5 mL, 1M in THF, 18.5 mmol) was added slowly and the resultant solution was warmed to room temperature gradually while stirring overnight. Anhydrous MeOH (0.75 mL, 16.49 mmol) was added at −10° C., the reaction stirred for 1 h at the same temperature and then for 1 h at room temperature. At this stage LCMS indicated the formation of 2-Methoxy-3-[2-(2,9,9-trimethyl-3,5-dioxa-4-bora-tricyclo[6.1.1.02,6]dec-4-yl)-2-(trimethylsilanyl-amino)-ethyl]-benzoic acid tert-butyl ester intermediate.

In a separate dry round bottom flask under argon containing 3-(tert-Butoxycarbonylamino-methyl)-benzoic acid (5.31 gm, 20.1 mmol), dry DCM (50 ml) was added. The contents in the flask were cooled to 0° C. N-Methylmorpholine (NMM, 2.77 mL, 25.2 mmol) was added followed by O-(7-Azabenzotriazol-1-yl)-N,N,N′,N′-tetramethyluronium hexafluorophosphate (HATU, 7.6 g, 20.1 mmol) and the mixture stirred for 30 min at 0° C. and then 1 hr at room temperature. To this reaction mixture was added all of the reaction mixture from Step 4 dropwise at −20° C. The cooling bath was removed and the reaction stirred at room temperature. After 2 h the reaction was quenched with H₂O (100 mL) and the aqueous phase was extracted with EtOAc (3×100 mL), the combined organic layers were dried over MgSO₄, and concentrated in vacuo. The crude product was purified by flash column chromatography [R_{f}=0.26, silica gel (EtOAc/Hexane, 30:70, v/v)] to give a 48% yield of product. ESI-MS m/z 663 (MH)⁺.

Step 5. Synthesis of 2(R)-3-[2-(3-(Aminomethyl)benzoylamino)-2-borono-ethyl]-2-hydroxy-benzoic acid hydrochloride. To a solution of 3-[2-[3-(tert-Butoxycarbonylamino-methyl)-benzoylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester (662 mg, 1.0 mmol) in DCM (15 ml) under argon was added BCl₃ (7 ml, 7 mmol, 1M solution in DCM) dropwise at −78° C. The mixture was stirred for 1 hr at the same temperature then warmed to 0° C. After 1 hr of stirring, the reaction was quenched with water (10 ml) at 0° C. The DCM layer was evaporated. More water (75 ml) was added, and the aqueous layer extracted with ether (3×50 ml). The aqueous layer was evaporated to 50 ml, the pH of aqueous layer was adjusted to 1.0, then it was purified on C18 reverse phase silica gel (Isopropanol (IPA)/H₂O, 2:98, v/v) to give 106 mg of resultant compound as a white solid in 31% yield. ESI-MS m/z 341 (MH−H₂O)⁺.

### Example 2

### 2(R)-3-[2-(4-(Aminomethyl)benzoylamino)-2-borono-ethyl]-2-hydroxy-benzoic acid hydrochloride

Step 1. Synthesis of 3-[2-[4-(tert-Butoxycarbonylamino-methyl)-benzoylamino]-2-(2,9,9-trimethyl-3,5-dioxa-4-bora-tricyclo[6.1.1.0^{2,6}]dec-4-yl)-ethyl]-2-hydroxy-benzoic acid. Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 4-(Boc-aminomethyl)benzoic acid following the procedure described in Step 4 in Example 1.

Step 2. Synthesis of 3-[2-(4-(Aminomethyl)-benzoylamino)-2-borono-ethyl]-2-hydroxy-benzoic acid hydrochloride. To a solution of 3-[2-[4-(tert-Butoxycarbonylamino-methyl)-benzoylamino]-2-(2,9,9-trimethyl-3,5-dioxa-4-bora-tricyclo[6.1.1.0^{2,6}]dec-4-yl)-ethyl]-2-hydroxy-benzoic acid (2.03 g, 3.1 mmole) in DCM (8 mL) at −78° C. was added BCl₃ (1.0M in DCM, 18 mL, 18 mmole). After stirring for 1.5 h at −78° C. the solution was allowed to warm to ca. −20° C. and then quenched with water. EtOAc was added and the layers separated. The aqueous layer was extracted with diethyl ether (Et₂O), and then the combined organic layers were washed once with water. The aqueous layers were then combined and extracted once with Et₂O, and once with EtOAc, then concentrated to ⅔ volume. After sitting overnight the cloudy solution was placed directly onto a C18 reverse phase silica gel column and eluted with a gradient of 100% water to 7% isopropanol/H₂O. The fractions obtained were a mixture of a monomeric form and dimeric form of product, and when combined showed a monomer/dimer ratio of 3/1. The combined fractions were concentrated to a volume=ca. 40 mL, and then the solution was taken to pH 14 by the addition of 1N NaOH. After stirring for 20 min, the dimer was fully converted to the monomer. The solution was acidified to pH 3 with 3N HCl and then placed on a second C18 reverse-phase silica gel column and elude with a gradient of 100% H₂O to 2% IPA/H₂O. The pure fractions were combined, concentrated in vacuo at 22-23° C. to about 5 mL and then freeze-dried overnight to afford 471 mg (39%) of an off-white solid as a 9/1 mixture of monomer/dimer. ESI-MS of monomer m/z 341 (MH−H₂O)+.

### Example 3

### 2(R)-3-[2-(4-(Morpholinomethyl)benzoylamino)-2-borono-ethyl]-2-hydroxy-benzoic acid formate

Step 1. Synthesis of 2-Methoxy-3-[2-((4-morpholin-4-ylmethyl)-benzoylamino)-2-(2,9,9-trimethyl-3,5-dioxa-4-bora-tricyclo[6.1.1.0^{2,6}]dec-4-yl)-ethyl]-benzoic acid tert-butyl ester. Prepared from 2-Methoxy-3-(2,9,9-trimethyl-3,5-dioxa-4-bora-tricyclo[6.1.1.0^{2,6}]dec-4-ylmethyl)-benzoic acid tert-butyl ester and 4-(Morpholinomethyl)benzoic acid following the procedure described in Step 4 of Example 1. The crude product was purified by flash column chromatography [R_{f}=0.23, silica gel (EtOAc 100%)] to give a 40% yield of the product. ESI-MS m/z 633 (MH)⁺.

Step 2. Synthesis of 2(R)-3-[2-(4-(Morpholinomethyl)benzoylamino)-2-borono-ethyl]-2-hydroxy-benzoic acid formate. Prepared from 2-Methoxy-3-[2-(4-morpholin-4-ylmethyl-benzoylamino)-2-(2,9,9-trimethyl-3,5-dioxa-4-bora-tricyclo[6.1.1.0^{2,6}]dec-4-yl)-ethyl]-benzoic acid tert-butyl ester and BCl₃, following the procedure described in Step 5 of Example 1. The crude product was purified by preparative reverse-phase HPLC using a water/acetonitrile gradient with 0.1% formic acid buffer to give 78 mg of resultant compound as a white solid in 23% yield. ESI-MS m/z 411 (MH−H₂O)⁺.

### Example 4

2(R)-3-[2-(4-(N,N-Dimethylaminomethyl)benzoylamino)-2-borono-ethyl]-2-hydroxy-benzoic acid formate salt

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 4-((dimethylamino)methyl)benzoic acid following the procedure described in Steps 4-5 of Example 1. The final product was purified by preparative HPLC using solvents buffered with 0.1% formic acid to afford the product as a white solid. ESI-MS m/z 369 (MH−H₂O)⁺. ¹H NMR (CD₃OD) δ 7.91-7.28 (m, 6H), 6.88 (m, 1H), 4.20 (m, 2H), 3.50 (m, 1H), 3.20-3.04 (m, 2H), 2.80-2.60 (m, 6H).

### Example 5

### 2(R)-3-[2-(4-(1-piperazinylmethyl)benzoylamino)-2-borono-ethyl]-2-hydroxy-benzoic acid Hydrochloride

Step 1. Synthesis of 2-Methoxy-3-[2-((4-bromomethyl)-benzoylamino)-2-(2,9,9-trimethyl-3,5-dioxa-4-bora-tricyclo[6.1.1.0^{2,6}]dec-4-yl)-ethyl]-benzoic acid tert-butyl ester. To anhydrous CH₂Cl₂ (2.4 mL, 37.5 mmol) in anhydrous THF (58 mL) under argon at −100° C. [MeOH, liq. N₂ slush bath], n-BuLi (12 mL, 2.5M in hexane, 30 mmol) was added dropwise and the mixture was stirred for 30 minutes. A THF (12 mL) solution of 2-Methoxy-3-(2,9,9-trimethyl-3,5-dioxa-4-bora-tricyclo[6.1.1.0^{2,6}]dec-4-ylmethyl)-benzoic acid tert-butyl ester (10 g, 25 mmol) was added over a period of 20 minutes. After 40 minutes the cooling bath was removed and the mixture warmed slowly to 0° C. After 2 hours the reaction flask was cooled to −78° C., LHMDS (30 mL, 1M in THF, 30 mmol) was added slowly and the resultant solution was warmed to room temperature gradually while stirring overnight. Anhydrous MeOH (1.21 mL, 30 mmol) was added at −10° C., the reaction stirred for 1 h at the same temperature and then for 1 h at room temperature. At this stage LCMS indicated the formation of 2-Methoxy-3-[2-(2,9,9-trimethyl-3,5-dioxa-4-bora-tricyclo[6.1.1.02,6]dec-4-yl)-2-(trimethylsilanyl-amino)-ethyl]-benzoic acid tert-butyl ester intermediate.

In a separate dry round bottom flask under argon containing 4-bromomethyl benzoyl bromide (5.0 gm, 18 mmol), dry DCM (50 ml) was added. The content in the flask were cooled to 0° C. To this reaction mixture was added (8.2 gm, 16.36 mmol) of reaction mixture from Step 1 dropwise at −20° C. The cooling bath was removed and the reaction stirred at room temperature. After 2 h the reaction was quenched with H₂O (100 mL) and the aqueous phase was extracted with EtOAc (3×100 mL), the combined organic layers were dried over Na₂SO₄, and concentrated in vacuo. The crude product was purified by flash column chromatography [R_{f}=0.5, silica gel (EtOAc/Hexane, 50:50, v/v)] to give a 48% yield of product. ESI-MS m/z 627 (MH)⁺.

Step 2. Synthesis of 2-Methoxy-3-[2-((4-Boc-piperazinylmethyl)-benzoylamino)-2-(2,9,9-trimethyl-3,5-dioxa-4-bora-tricyclo[6.1.1.0^{2,6}]dec-4-yl)-ethyl]-benzoic acid tert-butyl ester. To the solution of Boc-piperazine (0.075 g, 0.4 mmol) in CH₃CN:DMF (5:5 mL) was added 3-[2-(4-Bromoethyl-benzoylamino)-2-(2,9,9-trimethyl-3,5-dioxa-4-boratricycl[6.1.1.0^{2,6}]dec-4-yl)-ethyl]-2-methoxy-benzoic acid tert-butyl ester (0.250 g, 0.4 mmol). To the mixture was added Na₂CO₃ (0.045 g, 0.42 mmol) and the solution heated to 70° C. for 3 h. The reaction flask was cooled to room temperature, then extracted with ethyl acetate (3×50 mL). The ethyl acetate layers were washed with water three times and once with brine, dried over Na₂SO₄ and concentrated to give a crude product. ESI-MS m/z 732 (MH)⁺. The crude product was used without further purification.

Step 3. Synthesis of 2(R)-3-[2-(4-(1-piperazinylmethyl)benzoylamino)-2-borono-ethyl]-2-hydroxy-benzoic acid Hydrochloride. To a solution of 4-{4-[2-(3-tert-Butoxycarbonyl-2-methoxy-phenol)-1-(2,9,9-trimethyl-3,5-dioxa-4-bora-tricyclo[6.1.1.0^{2,6}]dec-4-yl)-ethylcarbamoyl]-benzyl}-piperazine-1-carboxylic acid tert-butylester (0.230 gm, 0.314 mmol) in DCM (15 ml) under argon was added BCl₃ (1.8 ml, 1.8 mmol, 1M solution in DCM) dropwise at −78° C. The mixture was stirred for 1 hr at the same temperature then warmed to 0° C. After 1 hr of stirring at 0° C., LCMS indicated the consumption of all of the starting material. At this point the reaction was quenched with water (10 ml) at 0° C. The DCM layer was evaporated in vacuo, more water (75 ml) was added and the aqueous layer extracted with ether (3×50 ml). The aqueous layer was evaporated to 50 ml, the pH was adjusted to 1.0, then it was purified on C18 reverse phase silica gel 90 (IPA/H₂O, 2:98, v/v) to give 90 mg of resultant compound as a white solid in 67% yield. ESI-MS m/z 411 (MH−H₂O)⁺.

### Example 6

### 2(R)-3-[2-(4-(N-ethylaminomethyl)benzoylamino)-2-borono-ethyl]-2-hydroxy-benzoic acid Hydrochloride

Prepared from 2-Methoxy-3-[2-((4-bromomethyl)-benzoylamino)-2-(2,9,9-trimethyl-3,5-dioxa-4-bora-tricyclo[6.1.1.0^{2,6}]dec-4-yl)-ethyl]-benzoic acid tert-butyl ester and ethylamine using the general procedure described in Steps 2 and 3 of Example 5.

### Example 7

### (2R)-3-{2-[(2-Amino-pyridine-5-carbonyl)-amino]-2-borono-ethyl}-2-hydroxy-benzoic acid formate salt

Step 1. Synthesis of 3-Borono-2-methoxybenzoic acid tert-butyl ester. To a solution of 3-borono-2-methoxybenzoic acid (Combi-blocks, 5.0 g, 25.5 mmole) in 1,4-dioxane (30 mL) in a sealed tube was added conc. H₂SO₄ (1.5 mL). The solution was cooled to 0° C. and an equal volume of 2-methylpropene was bubbled in. The tube was sealed and allowed to stir at ambient temperature for 18 h. The solution was cooled in an ice bath, the seal was opened and the solution stirred at ambient temperature for 30 min. The solution was basified with saturated aq. NaHCO₃ and extracted twice with EtOAc. The combined organic layers were washed with water (5×), brine, dried (Na₂SO₄) and concentrated in vacuo to afford 4.0 g (62%) of the product as a white solid. ESI-MS m/z 275 (M+Na)⁺.

Step 2. Synthesis of 2-Methoxy-3-(2,9,9-trimethyl-3,5-dioxa-4-bora-tricyclo[6.1.1.0^{2,6}]dec-4-yl)-benzoic acid tert-butyl ester. A solution of 3-borono-2-methoxybenzoic acid tert-butyl ester (4.0 g, 15.9 mmole), THF (21 mL), and (+)-pinanediol (2.70 g, 15.9 mmole) was stirred at room temperature for 1 h. The solution was concentrated in vacuo, and the residue chromatographed on SiO₂ with 6% EtOAc/hexane to afford 5.0 g (86%) of the product as a slowly crystallizing solid. ESI-MS m/z 409 (M+Na)⁺.

Step 3. Synthesis of 2-Methoxy-3-(2,9,9-trimethyl-3,5-dioxa-4-bora-tricyclo[6.1.1.0^{2,6}]dec-4-ylmethyl)-benzoic acid tert-butyl ester. A solution of 2-methoxy-3-(2,9,9-trimethyl-3,5-dioxa-4-bora-tricyclo[6.1.1.0^{2,6}]dec-4-yl)-benzoic acid tert-butyl ester (8.5 g, 22 mmol) and chloroiodomethane (4.6 g, 26.4 mmol) in THF (65 mL) under argon was cooled to −100° C. [MeOH, liq. N₂ slush bath]. n-BuLi (10.56 mL, 2.5M in hexane, 26.4 mmol) was added dropwise over a period of 10 minutes and the mixture stirred overnight. The reaction was quenched with H₂O (100 mL) and the aqueous phase was extracted with EtOAc (3×75 mL), the combined organic layers were dried over MgSO₄, and concentrated in vacuo. Purification by flash column chromatography on silica gel [R_{f}=0.21, (DCM/Hexane, 70:30, v/v)] afforded 8 g of the resultant compound as a colorless oil in 91% yield. ESI-MS m/z 401 (MH)⁺.

Step 4. Synthesis of 6-tert-Butoxycarbonylamino-nicotinic acid methyl ester. To a solution of methyl-2-amino-5-pyridinecarboxylate (10.1 g, 63.9 mmole) in a mixture of acetone (30 mL) and tert-butanol (89 mL) was added di-tert-butyl dicarbonate (21.0 g, 96.2 mmole) and N,N-dimethylaminopyridine (DMAP, 156 mg, 1.3 mmole). The solution was stirred for 20 h, and to the thick slurry was added 135 mL of hexane. The solution was cooled to −20° C. and stirred for 2 h. The solids were isolated by filtration, washed with cold 3/1 hexane/DCM and dried in vacuo to afford 12.8 g (78%) of product as a white solid.

Step 5. Synthesis of 6-tert-Butoxycarbonylamino-nicotinic acid. A solution of 6-tert-Butoxycarbonylamino-nicotinic acid methyl ester (5.0 g, 19.4 mmole), methanol (50 mL) and 1 N aqueous NaOH (40 mL, 40 mmole) was stirred for 20 h at room temperature and then heated to 60° C. for 2 h. The solution was cooled and the MeOH removed in vacuo. With stirring, 3N HCl was added to obtain a pH of 3 resulting in the precipitation of white solids. The solids were collected by filtration, washed with water and dried to afford 4.23 g (89%) of white solids.

Step 6. Synthesis of 3-[2-[(6-tert-Butoxycarbonylamino-pyridine-3-carbonyl)-amino]-2-(2,9,9-trimethyl-3,5-dioxa-4-bora-tricyclo[6.1.1.0^{2,6}]dec-4-yl)-ethyl]-2-methoxy-benzoic acid tert-butyl ester. To anhydrous CH₂Cl₂ (0.41 mL, 6.5 mmol) in anhydrous THF (16 mL) under argon at −100° C. [MeOH, liq. N₂ slush bath], n-BuLi (2.4 mL, 2.5M in hexane, 6.0 mmol) was added dropwise and the mixture was stirred for 30 minutes. A THF (12 mL) solution of 2-Methoxy-3-(2,9,9-trimethyl-3,5-dioxa-4-bora-tricyclo[6.1.1.0^{2,6}]dec-4-ylmethyl)-benzoic acid tert-butyl ester (2.0 g, 5.0 mmol) was added over a period of 20 minutes. After 40 minutes the cooling bath was removed and the mixture warmed slowly to 0° C. After 2 hours the reaction flask was cooled to −78° C., LHMDS (5.5 mL, 1M in THF, 5.5 mmol) was added slowly and the resultant solution was warmed to room temperature gradually while stirring overnight. Anhydrous MeOH (0.22 mL, 5.5 mmol) was added at −10° C., the reaction stirred for 1 h at the same temperature and then for 1 h at room temperature. At this stage LCMS indicated the formation of 2-Methoxy-3-[2-(2,9,9-trimethyl-3,5-dioxa-4-bora-tricyclo[6.1.1.02,6]dec-4-yl)-2-(trimethylsilanyl-amino)-ethyl]-benzoic acid tert-butyl ester intermediate.

In a separate dry round bottom flask under argon containing 6-tert-Butoxycarbonylamino-nicotinic acid (1.43 gm, 6.0 mmol), dry DCM (20 ml) was added. The contents in the flask were cooled to 0° C. NMM (0.71 mL, 6.5 mmol) was added followed by HATU (2.28 g, 6.0 mmol) and DMF (10 ml). The mixture stirred for 30 min at 0° C. and then 1 hr at room temperature. To this reaction mixture was added all of the reaction mixture from Step 1 dropwise at −10° C. The cooling bath was removed and the reaction stirred at room temperature. After 2 h the reaction was quenched with H₂O (100 mL) and the aqueous phase was extracted with EtOAc (3×100 mL), the combined organic layers were dried over MgSO₄, and concentrated in vacuo. The crude product was purified by flash column chromatography [R_{f}=0.15, silica gel (EtOAc/Hexane, 30:70, v/v)] to give a 35% yield of product. ESI-MS m/z 650 (MH)⁺.

Step 7. (2R)-3-{2-[(2-Amino-pyridine-5-carbonyl)-amino]-2-borono-ethyl}-2-hydroxy-benzoic acid formate salt. To a solution of 3-[2-[(6-tert-Butoxycarbonylamino-pyridine-3-carbonyl)-amino]-2-(2,9,9-trimethyl-3,5-dioxa-4-bora-tricyclo[6.1.1.0^{2,6}]dec-4-yl)-ethyl]-2-methoxy-benzoic acid tert-butyl ester (300 mg, 0.46 mmol) in DCM (10 ml) under argon was added BCl₃ (3.7 ml, 3.7 mmol, 1M solution in DCM) drop wise at −78° C. The mixture was stirred for 1 hr at the same temperature then warmed to 0° C. After 1 hr of stirring at 0° C., LCMS indicated the consumption of all of the starting material. At this point the reaction was quenched with water (10 ml) at 0° C. The DCM layer was evaporated. More water (75 ml) was added and the aqueous layer extracted with ether (3×50 ml). The aqueous layer was evaporated and the crude product was purified by preparative HPLC using solvents buffered with formic acid to give 50 mg of resultant compound as a white solid in 31% yield. ESI-MS m/z 328 (MH−H₂O)⁺.

### Example 8

### (2R)-3-{2-[(2-Amino-pyridine-4-carbonyl)-amino]-2-borono-ethyl}-2-hydroxy-benzoic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and methyl-2-aminopyridine-4-carboxylate using the general procedure described in Steps 4-7 of Example 7. ESI-MS m/z 328 (MH−H₂O)⁺.

### Example 9

### (2R)-3-{2-[(2-(1-piperazinyl)-pyridine-5-carbonyl)-amino]-2-borono-ethyl}-2-hydroxy-benzoic acid hydrochloride

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 4-(5-Carboxy-pyridin-2-yl)-piperazine-1-carboxylic acid tert-butyl ester using the general procedure described in Steps 6-7 of Example 7. ESI-MS m/z 397 (MH−H₂O)+.

### Example 10

### (2R)-3-{2-[(2-(acetylamino)-pyridine-5-carbonyl)-amino]-2-borono-ethyl}-2-hydroxy-benzoic acid

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 6-Acetylamino-nicotinic acid using the general procedure described in Steps 6-7 of Example 7. ESI-MS m/z 370 (MH−H₂O)+.

### Example 11

### (1R)-[4-(methylamino)methyl]-benzoylamino-(3-carboxy-2-hydroxy)benzyl-methyl boronic acid hydrochloride

Step 1. Synthesis of 3-borono-2-methoxybenzoic acid tert-butyl ester. To a solution of 3-borono-2-methoxybenzoic acid (Combi-blocks, 5.0 g, 25.5 mmole) in 1,4-dioxane (30 mL) in a sealed tube was added conc. H₂SO₄ (1.5 mL). The solution was cooled to 0° C., and an equal volume of 2-methylpropene was bubbled in. The tube was sealed and allowed to stir at ambient temperature for 18 h. The solution was cooled in an ice bath, the seal was opened and the solution stirred at ambient temperature for 30 min. The solution was basified with saturated aq. NaHCO₃ and extracted twice with ethyl acetate (EtOAc). The combined organic layers were washed with water (5×), brine, dried (Na₂SO₄) and concentrated in vacuo to afford 4.0 g (62%) of the product as a white solid. ESI-MS m/z 275 (M+Na)⁺.

Step 2. Synthesis of 2-methoxy-3-(2,9,9-trimethyl-3,5-dioxa-4-bora-tricyclo[6.1.1.0^{2,6}]dec-4-yl)-benzoic acid tert-butyl ester. A solution of 3-borono-2-methoxybenzoic acid tert-butyl ester (4.0 g, 15.9 mmole), tetrahydrofuran (THF, 21 mL), and (+)-pinanediol (2.70 g, 15.9 mmole) was stirred at room temperature for 1 h. The solution was concentrated in vacuo, and the residue chromatographed on SiO₂ with 6% EtOAc/hexane to afford 5.0 g (86%) of the product as a slowly crystallizing solid. ESI-MS m/z 409 (M+Na)⁺.

Step 3. Synthesis of 2-methoxy-3-(2,9,9-trimethyl-3,5-dioxa-4-bora-tricyclo [6.1.1.0^{2,6}]dec-4-ylmethyl)-benzoic acid tert-butyl ester. A solution of 2-methoxy-3-(2,9,9-trimethyl-3,5-dioxa-4-bora-tricyclo[6.1.1.0^{2,6}]dec-4-yl)-benzoic acid tert-butyl ester (8.5 g, 22 mmol) and chloroiodomethane (4.6 g, 26.4 mmol) in THF (65 mL) under argon was cooled to −100° C. [MeOH, liq. N₂ slush bath]. n-Butyllithium (n-BuLi, 10.56 mL, 2.5M in hexane, 26.4 mmol) was added dropwise over a period of 10 minutes and the mixture stirred overnight. The reaction was quenched with H₂O (100 mL) and the aqueous phase was extracted with EtOAc (3×75 mL), the combined organic layers were dried over MgSO₄, and concentrated in vacuo. Purification by flash column chromatography on silica gel [R_{f}=0.21, (dichloromethane (DCM)/Hexane, 70:30, v/v)] afforded 8 g of the resultant compound as a colorless oil in 91% yield. ESI-MS m/z 401 (MH)⁺.

Step 4. Synthesis of 3-[2-(4-bromomethyl-benzoylamino)-2-(2,9,9-trimethyl-3,5-dioxa-4-boratricycl[6.1.1.0^{2,6}]dec-4-yl)-ethyl]-2-methoxy-benzoic acid tert-butyl ester. To anhydrous CH₂Cl₂ (2.4 mL, 37.5 mmol) in anhydrous THF (58 mL) under argon at −100° C. [MeOH, liq. N₂ slush bath], n-BuLi (12 mL, 2.5M in hexane, 30 mmol) was added dropwise and the mixture was stirred for 30 minutes. A THF (12 mL) solution of 2-methoxy-3-(2,9,9-trimethyl-3,5-dioxa-4-bora-tricyclo[6.1.1.0^{2,6}]dec-4-ylmethyl)-benzoic acid tert-butyl ester (10 g, 25 mmol) was added over a period of 20 minutes. After 40 minutes the cooling bath was removed and the mixture warmed slowly to 0° C. After 2 hours the reaction flask was cooled to −78° C., lithium hexamethyldisilazide (LHMDS) (30 mL, 1M in THF, 30 mmol) was added slowly and the resultant solution was warmed to room temperature gradually while stirring overnight. Anhydrous MeOH (1.21 mL, 30 mmol) was added at −10° C., the reaction stirred for 1 h at the same temperature and then for 1 h at room temperature. At this stage liquid chromatography mass spectrometry (LCMS) indicated the formation of 2-methoxy-3-[2-(2,9,9-trimethyl-3,5-dioxa-4-bora-tricyclo[6.1.1.02,6]dec-4-yl)-2-(trimethylsilanyl-amino)-ethyl]-benzoic acid tert-butyl ester intermediate.

In a separate dry round bottom flask under argon containing 4-bromomethyl benzoyl bromide (5.0 gm, 18 mmol), dry DCM (50 ml) was added. The content in the flask was cooled to 0° C. To this reaction mixture was added (73 mL, 16.4 mmol) of reaction mixture from Step 1 dropwise at −20° C. The cooling bath was removed and the reaction stirred at room temperature. After 2 h the reaction was quenched with H₂O (100 mL) and the aqueous phase was extracted with EtOAc (3×100 mL), the combined organic layers were dried over Na₂SO₄, and concentrated in vacuo. The crude product was purified by flash column chromatography [R_{f}=0.5, silica gel (EtOAc/Hexane, 50:50, v/v)] to give a 48% yield of product. ESI-MS m/z 627 (MH)⁺.

Step 5. Synthesis of 2-methoxy-3-[2-(4-methylaminoethyl-benzoylamino)-2-(2,9,9-trimethyl-3,5-dioxa-4-bora-tricyclo[6.1.1.0^{2,6}]dec-4-yl)-ethyl]-benzoic acid tert-butyl ester. To a solution of 3-[2-(4-bromomethyl-benzoylamino)-2-(2,9,9-trimethyl-3,5-dioxa-4-boratricycl[6.1.1.0^{2,6}]dec-4-yl)-ethyl]-2-methoxy-benzoic acid tert-butyl ester (0.4 g, 0.64 mmol) in acetonitrile (10 mL), methylamine (0.4 mL, 0.76 mmol) and sodium carbonate (0.112 g, 1.06 mmol) added and stirred at room temperature for 3 hrs. The solvent was evaporated to dryness, the residue redissolved in ethyl acetate, water was added and extracted with ethyl acetate. The organic layer was washed with water and brine, dried over sodium sulphate and concentrated on a rotary evaporator. The material was used in the next step without purification.

Step 6. Synthesis of (1R)-[4-methylaminomethyl)benzoylamino-(3-carboxy-2-hydroxy)benzyl-methyl boronic acid hydrochloride. To 2-methoxy-3-[2-(4-methylaminoethyl-benzoylamino)-2-(2,9,9-trimethyl-3,5-dioxa-4-bora-tricyclo[6.1.1.0^{2,6}]dec-4-yl)-ethyl]-benzoic acid tert-butyl ester (0.300 gm, 0.52 mmol) in DCM (3 mL) at −78° C., BCl₃ (2.6 mL, 2.6 mmol, 1M solution in DCM) was added dropwise and the mixture stirred for 1 hr at the same temperature then warmed to 0° C. After 1 hr of stirring at 0° C., LCMS indicated the consumption of all of the starting material. At this point the reaction was quenched with water (10 ml) at 0° C. The DCM layer was evaporated. More water (75 ml) was added and the aqueous layer extracted with ether (3×50 ml). The aqueous layer was evaporated to 50 ml, the pH of the aqueous layer was adjusted to 1.0, then it was purified on C18 reverse phase silica gel (IPA/H₂O, 2:98, v/v) to give 77 mg of resultant compound as a white solid in 40% yield. ESI-MS m/z 355 (MH−H₂O)⁺.

### Example 12

### (1R)-[4-(piperidine-3-carboxylic acid)-methyl]-benzoylamino-(3-carboxy-2-hydroxy)benzyl-methyl boronic acid hydrochloride

Step 1. Synthesis of 1-{4[2-(3-tert-butoxycarbonyl-2-methoxy-phenyl)-1-(2,9,9-trimethyl-3,5-dioxa-4-bora-tricyclo[6.1.1.0^{2,6}]dec-4-yl)-ethyl-carbamoyl]-benzyl}piperidine-3-caroxylic acid ethyl ester. To a solution of 3-[2-(4-bromomethyl-benzoylamino)-2-(2,9,9-trimethyl-3,5-dioxa-4-boratricycl[6.1.1.0^{2,6}]dec-4-yl)-ethyl]-2-methoxy-benzoic acid tert-butyl ester (0.3 g, 0.48 mmol) in acetonitrile (10 mL), ethyl nipecoate (0.076 g, 0.48 mmol) and sodium carbonate (0.061 g, 0.57 mmol) were added and the reaction stirred at room temperature for 1 hr. The solvent was evaporated to dryness, the residue redissolved in ethyl acetate, water added, and extracted with ethyl acetate. The organic layer was washed with water and brine, dried over sodium sulphate and concentrated on a rotary evaporater. The material was used in the next step without purification.

Step 2. Synthesis of (1R)-[4-(piperidine-3-carboxylic acid)-methyl]-benzoylamino-(3carboxy-2-hydroxy)benzyl-methyl boronic acid hydrochloride. To 1-{4[2-(3-tert-Butoxycarbonyl-2-methoxy-phenyl)-1-(2,9,9-trimethyl-3,5-dioxa-4-bora-tricyclo[6.1.1.0^{2,6}]dec-4-yl)-ethyl carbamoyl]-benzyl}piperidine-3-caroxylic acid ethyl ester (0.270 gm, 0.38 mmol) in DCM (3 mL) at −78° C. was added BCl₃ (2.3 mL, 2.3 mmol, 1M in DCM) and stirred for 3 hrs, (during this time all protecting groups deprotected except ester group) without adding additional dry ice in the bath. The reaction was then warmed to 0° C. and stirred for 2 hrs. Water was added and the DCM evaporated. The mixture was extracted with ethyl acetate and the aqueous layer basified with NaOH and stirred at room temperature for 2 hr. The solution was acidified with 1N HCl and purified on C18 reverse phase silica gel 90 (IPA/H₂O, 2:98, v/v) to give 100 mg of resultant compound as a white solid in 55% yield. ESI-MS m/z 453 (MH−H₂O)⁺.

### Example 13

### (1R)-(4-(triethylammonium)methyl)-benzoylamino-(3-carboxy-2-hydroxy)benzyl-methyl boronic acid bromide

Step 1. Synthesis of 4-[2-(3-tert-butoxycarbonyl-2-methoxy-phenyl)-1-(2,9,9-trimethyl-3,5-dioxa-4-bora-tricyclo[6.1.1.0^{2,6}]dec-4-yl)-ethylcarbamoyl]-benzyl]-triethyl-ammonium bromide. To a solution of 3-[2-(4-bromomethyl-benzoylamino)-2-(2,9,9-trimethyl-3,5-dioxa-4-boratricycl[6.1.1.0^{2,6}]dec-4-yl)-ethyl]-2-methoxy-benzoic acid tent-butyl ester (0.3 g, 0.48 mmol) in dichloromethane (10 mL), was added triethylamine (0.06 mL, 0.44 mmol) and stirred at room temperature overnight. The solvent was evaporated and the crude product taken to next step.

Step 2. Synthesis of (1R)-(4-(triethyl amino)-methyl)-benzoylamino-(3-carboxy-2-hydroxy)benzyl-methyl boronic acid bromide. To a solution of {4-[2-(3-tert-Butoxycarbonyl-2-methoxy-phenyl)-1-(2,9,9-trimethyl-3,5-dioxa-4-bora-tricyclo[6.1.1.0^{2,6}]dec-4-yl)-ethylcarbamoyl]-benzyl]-triethyl-ammonium bromide (0.2 g, 0.309 mmol) in DCM (3 mL) at −78° C. was added BCl₃ (1.25 mL, 1.23 mmol) and stirred for 2 hrs. LCMS confirmed completion of the reaction. At this point the reaction was quenched with water (10 ml) at 0° C. The DCM layer was evaporated, more water (75 ml) was added and the aqueous layer extracted with ether (3×50 ml). The aqueous layer was evaporated to 50 ml, the pH of the aqueous layer was adjusted to 1.0, then it was purified on C18 reverse phase silica gel 90 (IPA/H₂O, 2:98, v/v) to give 28 mg of resultant compound as a white solid in 20% yield. ESI-MS m/z 425 (MH−H₂O)⁺.

### Example 14

### (1R)-[4-(pyridinium)-methyl]-benzoylamino-(3-carboxy-2-hydroxy)benzyl-methyl boronic acid chloride

Step 1. Synthesis of 4-[2-(3-tert-butoxycarbonyl-2-methoxy-phenyl)-1-(2,9,9-trimethyl-3,5-dioxa-4-bora-tricyclo[6.1.1.0^{2,6}]dec-4-yl)-ethylcarbamoyl]-benzyl]-pyridinium bromide. To a solution of 3-[2-(4-bromomethyl-benzoylamino)-2-(2,9,9-trimethyl-3,5-dioxa-4-boratricycl[6.1.1.0^{2,6}]dec-4-yl)-ethyl]-2-methoxy-benzoic acid tent-butyl ester (0.2 g, 0.31 mmol) in DCM (5 mL), pyridine (0.025 mL, 0.31 mmol) was added and stirred at room temperature overnight. Water was added and the mixture extracted with DCM. The organic layer was washed with brine, dried over sodium sulphate and concentrated on rotary evaporater. The material was used in the next step without purification.

Step 2. (1R)-[4-(pyridinium)-methyl]-benzoylamino-(3-carboxy-2-hydroxy)benzyl-methyl boronic acid chloride. To a solution of {4-[2-(3-tert-Butoxycarbonyl-2-methoxy-phenyl)-1-(2,9,9-trimethyl-3,5-dioxa-4-bora-tricyclo[6.1.1.0^{2,6}]dec-4-yl)-ethylcarbamoyl]-benzyl}-pyridium bromide (0.25 g, 0.4 mmol) in DCM (3 mL) at −78° C. was added BCl₃ (2 mL, 2 mmol) and stirred for 2 hrs. LCMS confirmed completion of the reaction. At this point the reaction was quenched with water (10 ml) at 0° C. The DCM layer was evaporated, more water (75 ml) was added and the aqueous layer extracted with ether (3×50 ml). The aqueous layer was evaporated to 50 ml, the pH of the aqueous layer was adjusted to 1.0, then it was purified on C18 reverse phase silica gel 90 (IPA/H₂O, 2:98, v/v) to give 32 mg of resultant compound as a white solid in 21% yield. ESI-MS m/z 404 (MH−H₂O)⁺.

### Example 15

### (1R)-[4-(2-amino-ethyl amino)-methyl]-benzoylamino-(3-carboxy-2-hydroxy)benzyl-methyl boronic acid hydrochloride

Step 1. Synthesis of 4-[2-(3-tert-butoxycarbonyl-2-methoxy-phenyl)-1-(2,9,9-trimethyl-3,5-dioxa-4-bora-tricyclo[6.1.1.0^{2,6}]dec-4-yl)-ethylcarbamoyl]-benzyl]-2-tert-Butoxycarbonyl amino-ethylamine. To a solution of 3-[2-(4-bromomethyl-benzoylamino)-2-(2,9,9-trimethyl-3,5-dioxa-4-boratricycl[6.1.1.0^{2,6}]dec-4-yl)-ethyl]-2-methoxy-benzoic acid tert-butyl ester (0.3 g, 0.48 mmol) in DCM (5 mL), N-BOC ethylenediamine (0.076 mL, 0.48 mmol) was added and the reaction stirred at room temperature for overnight. Water was added and the solution extracted with DCM. The organic layer was washed with brine, dried over sodium sulphate and concentrated on rotary evaporator. The crude product was purified by flash chromatography to afford 70 mg (20% yield) of product.

Step 2. Synthesis of (1R)-[4-(2-amino-ethyl amino)-methyl]-benzoylamino-(3-carboxy-2-hydroxy)benzyl-methyl boronic acid hydrochloride. To a solution {4-[2-(3-tert-butoxycarbonyl-2-methoxy-phenyl)-1-(2,9,9-trimethyl-3,5-dioxa-4-bora-tricyclo[6.1.1.0^{2,6}]dec-4-yl)-ethylcarbamoyl]-benzyl}-2-tert-butoxycarbonyl amino-ethylamine (0.075 g, 0.106 mmol) in DCM (5 mL) at −78° C. was added BCl₃ (0.53 mL, 0.53 mmol) and the reaction stirred for 2 hrs. LCMS confirmed completion of the reaction. At this point the reaction was quenched with water (10 ml) at 0° C. The DCM layer was evaporated, more water (75 ml) was added and the aqueous layer extracted with ether (3×50 ml). The aqueous layer was evaporated to 50 ml, the pH of the aqueous layer was adjusted to 1.0, then it was purified on C18 reverse phase silica gel 90 (IPA/H₂O, 2:98, v/v) to give 3.1 mg of resultant compound as a white solid in 7.4% yield. ESI-MS m/z 384 (MH−H₂O)⁺.

### Example 16

### (1R)-[4-(2-hydroxy-ethylamino)-methyl]-benzoylamino-(3-carboxy-2-hydroxy)benzyl-methyl boronic acid hydrochloride

Step 1. Synthesis of 3-[2-{4-[2-hydroxy-ethylamino)-methyl]-benzoylamino}2-(2,9,9-trimethyl-3,5-dioxa-4-bora-tricyclo[6.1.1.0^{2,6}]dec-4-yl)-ethyl]-2-methoxy-benzoic acid tert-butyl ester. To a solution of 3-[2-(4-bromomethyl-benzoylamino)-2-(2,9,9-trimethyl-3,5-dioxa-4-boratricycl[6.1.1.0^{2,6}]dec-4-yl)-ethyl]-2-methoxy-benzoic acid tent-butyl ester (0.3 g, 0.48 mmol) in DCM (3 mL) was added ethanolamine (0.03 mL, 0.48 mmol) and stirred overnight at room temperature. The DCM was evaporated, the residue dissolved in ethyl acetate and washed with water and brine, dried over sodium sulphate and evaporated to dryness on rotary evaporater. The material was used in the next step without purification.

Step 2. Synthesis of (1R)-(4-(2-hydroxy-ethylamino)-methyl]-benzoylamino-(3-carboxy-2-hydroxy)benzyl-methyl boronic acid hydrochloride. To a solution 3-[2-{4-[2-Hydroxy-ethylamino)-methyl]-benzoylamino}2-(2,9,9-trimethyl-3,5-dioxa-4-bora-tricyclo[6.1.1.0^{2,6}]dec-4-yl)-ethyl]-2-methoxy-benzoic acid tert-butyl ester (0.100 g, 0.165 mmol) in DCM (10 mL) at −78° C. was added BCl₃ (0.66 mL, 0.66 mmol) and the mixture stirred for 2 hrs. LCMS confirmed completion of the reaction. At this point the reaction was quenched with water (10 ml) at 0° C. The DCM layer was evaporated, more water (75 ml) was added and the aqueous layer extracted with ether (3×50 ml). The aqueous layer was evaporated to 50 ml, the pH of aqueous layer was adjusted to 1.0, then it was purified on C18 reverse phase silica gel 90 (IPA/H₂O, 2:98, v/v) to give 2.7 mg of resultant compound as a white solid in 4% yield. ESI-MS m/z 385 (MH−H₂O)⁺.

### Example 17

### (1R)-[4-(pyridin-3-ylmethylaminomethyl]-benzoylamino-(3-carboxy-2-hydroxy) benzyl-methyl boronic acid hydrochloride

Step 1. Synthesis of 2-methoxy-3-[2-(4-{[pyridin-3-ylmethyl-amino]-methyl}-benzoylamino}2-(2,9,9-trimethyl-3,5-dioxa-4-bora-tricyclo[6.1.1.0^{2,6}]dec-4-yl)-ethyl]-benzoic acid tert-butyl ester. To a solution of 3-[2-(4-bromomethyl-benzoylamino)-2-(2,9,9-trimethyl-3,5-dioxa-4-boratricycl[6.1.1.0^{2,6}]dec-4-yl)-ethyl]-2-methoxy-benzoic acid tert-butyl ester (0.450 g, 0.718 mmol) in DCM (3 mL) was added 3-methylaminopyridine (0.093 mg, 0.86 mmol) and stirred over night at room temperature. The DCM was evaporated, the residue dissolved in ethyl acetate, washed with water and brine, dried over sodium sulphate and evaporated to dryness on rotary evaporater. The material was used in the next step without purification.

Step 2. Synthesis of (1R)-(4-(pyridin-3-ylaminomethyl]-benzoylamino-(3-carboxy-2-hydroxy)benzyl-methyl boronic acid hydrochloride. To a solution 2-methoxy-3-[2-(4-{[pyridin-3-ylmethyl-amino]-methyl}-benzoylamino}2-(2,9,9-trimethyl-3,5-dioxa-4-bora-tricyclo[6.1.1.0^{2,6}]dec-4-yl)-ethyl]-benzoic acid tert-butyl ester (0.500 g, 0.76 mmol) in DCM (10 mL) at −78° C. was added BCl₃ (3.8 mL, 3.82 mmol) and the mixture stirred for 2 hrs. LCMS confirmed completion of the reaction. At this point the reaction was quenched with water (10 ml) at 0° C. The DCM layer was evaporated, more water (75 ml) was added and the aqueous layer extracted with ether (3×50 ml). The aqueous layer was evaporated to 50 ml, the pH of the aqueous layer was adjusted to 1.0, then it was purified on C18 reverse phase silica gel 90 (IPA/H₂O, 2:98, v/v) to give 40 mg of resultant compound as a white solid in 11% yield. ESI-MS m/z 432 (MH−H₂O)⁺.

### Example 18

### (1R)-[3-{(2-aminoethylamino)-methyl}-benzoylamino]-(3-carboxy-2-hydroxy)benzyl-methylboronic acid formate

Step 1. Synthesis of 3-[2-(3-formyl-benzoylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester. To anhydrous CH₂Cl₂ (2.5 mL, 38.9 mmol) in anhydrous THF (95 mL) under argon at −100° C. [MeOH, liq. N₂ slush bath], n-BuLi (14.4 mL, 2.5M in hexane, 35.9 mmol) was added dropwise and the mixture was stirred for 30 minutes. A THF (20 mL) solution of 2-methoxy-3-(2,9,9-trimethyl-3,5-dioxa-4-bora-tricyclo[6.1.1.0^{2,6}]dec-4-ylmethyl)-benzoic acid tert-butyl ester (12.0 g, 30.0 mmol) was added over a period of 20 minutes. After 40 minutes the cooling bath was removed and the mixture warmed slowly to 0° C. After 1 hour the reaction flask was cooled to −78° C., LHMDS (33.0 mL, 1M in THF, 33.0 mmol) was added slowly and the resultant solution was warmed to room temperature gradually while stirring overnight. Anhydrous MeOH (1.33 mL, 33.0 mmol) was added at −10° C., the reaction stirred for 1 h at the same temperature and then for 1 h at room temperature. At this stage LCMS indicated the formation of 2-methoxy-3-[2-(2,9,9-trimethyl-3,5-dioxa-4-bora-tricyclo[6.1.1.0^{2,6}]dec-4-yl)-2-(trimethylsilanyl-amino)-ethyl]-benzoic acid tert-butyl ester intermediate.

To a separate dry round bottom flask under argon containing DMF (2.77 ml, 36.0 mmol) in Et₂O (100 ml) at 0° C., oxalyl chloride (3.14 ml, 36.0 mmol) was added with constant stirring. Gas evolution ceased in ca. 5 minutes, and a colourless precipitate formed. Ether was evaporated under vacuum, and to the solid salt so obtained was added DCM (60 ml). The suspension was cooled to −20° C. and 3-formyl-benzoic acid (5.4 g, 36.0 mmol) was added at once. In less than 5 minutes, all of the material went in solution, indicating that 3-formyl-benzoylchloride has formed. To the previously prepared solution of 2-methoxy-3-[2-(2,9,9-trimethyl-3,5-dioxa-4-bora-tricyclo[6.1.1.0^{2,6}] dec-4-yl)-2-(trimethylsilanyl-amino)-ethyl]-benzoic acid tert-butyl ester intermediate at −78° C. was added pyridine (6.78 ml, 83.9 mmol) followed by freshly prepared solution of 3-formyl-benzoylchloride. After stirring for 45 minutes at the same temperature, the cooling bath was removed and the reaction stirred at room temperature. After 2 h the reaction was quenched with H₂O (150 mL) and the aqueous phase was extracted with EtOAc (3×100 mL), the combined organic layers were washed with saturated NaHCO₃ (40 ml) followed by brine (50 ml) and then dried over MgSO₄, and concentrated in vacuo. The crude product was purified by flash column chromatography [R_{f}=0.25, silica gel (EtOAc/Hexane, 40:60, v/v)] to give 4.7 g of the coupled product in 24% yield. ESI-MS m/z 562 (MH)⁺.

Step 2. Synthesis of 3-[2-{3-[(2-tert-butoxycarbonylamino-ethylamino)-methyl]-benzoylamino}-2-(2,9,9-trimethyl-3,5-dioxa-4-bora tricyclo[6.1.1.0^{2,6}] dec-4-yl)-ethyl]-2-methoxy-benzoic acid tert-butyl ester. To a solution of 3-[2-(3-formyl-benzoylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester (400 mg, 0.71 mmol) in MeOH (4 ml) was added N-Boc-ethylenediamine (126 mg, 0.78 mmol) followed by AcOH (47 mg, 0.78 mmol) and NaCNBH₃ (89 mg, 1.42 mmol). After stirring the reaction mixture for 2 hours at room temperature LCMS indicated the complete consumption of starting material. Solvent was removed under vacuum, water (20 ml) was added and the residues were extracted with EtOAc (3×50 mL), the combined organic layers were washed with saturated NaHCO₃ (10 ml) followed by brine (15 ml) and then dried over MgSO₄, and concentrated in vacuo. The crude product was purified by flash column chromatography [R_{f}=0.14 silica gel (MeOH/DCM, 05:95, v/v)] to give 290 mg of the desired product in 58% yield. ESI-MS m/z 706 (MH)⁺.

Step 3. Synthesis of (1R)-(3-{(2-amino-ethylamino)-methyl}-benzoylamino]-(3-carboxy-2-hydroxy)benzyl-methylboronic acid formate. To a solution of 3-[2-{3-[(2-tert-butoxycarbonylamino-ethylamino)-methyl]-benzoylamino}-2-(2,9,9-trimethyl-3,5-dioxa-4-bora tricyclo[6.1.1.0^{2,6}]dec-4-yl)-ethyl]-2-methoxy-benzoic acid tert-butyl ester (290 mg, 0.41 mmol) in DCM (4 ml) under argon was added BCl₃ (3.7 ml, 3.7 mmol, 1M solution in DCM) dropwise at −78° C. Mixture stirred for 1 hr at the same temperature then warmed to 0° C. After 1 hr of stirring at 0° C., LCMS indicated the consumption of all of the starting material. At this point the reaction was quenched with water (10 ml) at 0° C. The DCM layer was evaporated, more water (60 ml) was added and the aqueous layer extracted with ether (3×40 ml). The aqueous layer was evaporated to 15 ml, the pH of the aqueous layer was adjusted to 1.0, then it was purified by preparative HPLC to give 72 mg of resultant compound as a white solid in 39% yield. ESI-MS m/z 384 (MH−H₂O)⁺.

### Example 19

### (1R)-(3-{[(pyridin-3-ylmethyl)amino]methyl}-benzoylamino)-(3-carboxy-2-hydroxy)benzyl-methylboronic acid formate

Step 1. Synthesis of 3-[(tert-butoxycarbonyl-pyridin-3-ylmethyl-amino)-methyl]-benzoic acid. To a solution of 3-formyl benzoic acid (2.0 g, 13.32 mmol) in MeOH (25 ml) was added 3-aminomethylpyridine (1.44 g, 13.32 mmol) and AcOH (0.8 g, 13.32 mmol). NaCNBH₃ (1.05 gm, 16.0 mmol) was then added at room temperature. At this point the reaction mixture became cloudy and it remained cloudy during the course of the reaction. After stirring the reaction mixture for 2 hours at room temperature, LCMS indicated the complete consumption of starting material and formation of the 3-{[(pyridin-3-ylmethyl)-amino]-methyl}-benzoic acid product. ESI-MS m/z 243 (MH)⁺. The solvent was removed under vacuum. The residue was dissolved in water (50 ml), NaOH (0.8 gm, 20 mmol) and Boc₂O (4.36 gm, 20 mmol) were added and the mixture stirred for 2 hours. The reaction was quenched with 1N HCl to get pH=5.5 and extracted with EtOAc (3×60 mL), the combined organic layers were dried over MgSO₄, and concentrated in vacuo. The crude product which was formed in 40% yield was taken to next stage without purification. ESI-MS m/z 343 (MH)⁺.

Step 2. Synthesis of 2-methoxy-3-[2-(3-{[(pyridin-3-ylmethyl)-amino]-methyl}-benzoylamino)-2-(2,9,9-trimethyl-3,5-dioxa-4-bora-tricyclo [6.1.1.02^{2,6}]dec-4-yl)-ethyl]-benzoic acid tert-butyl ester. 2-methoxy-3-[2-(2,9,9-trimethyl-3,5-dioxa-4-bora-tricyclo[6.1.1.0^{2,6}]dec-4-yl)-2-(trimethylsilanyl-amino)-ethyl]-benzoic acid tert-butyl ester intermediate was made according to the procedure described in Step 1 of Example 18 on a 3.5 mmol scale. In a separate dry round bottom flask under argon containing 3-[(tert-butoxycarbonyl-pyridin-3-ylmethyl-amino)-methyl]-benzoic acid (1.31 gm, 3.84 mmol), dry DCM (10 ml) was added. The contents in the flask were cooled to 0° C. N-methylmorpholine (NMM) (530 mg, 5.24 mmol) was added followed by O-(7-azabenzotriazol-1-yl)-N,N,N′,N′-tetramethyluronium hexafluorophosphate (HATU) (1.46 g, 3.84 mmol) and DMF (10 ml), the mixture stirred for 30 min at 0° C. and then 1 hr at room temperature. To this reaction mixture was added the above-described 2-methoxy-3-[2-(2,9,9-trimethyl-3,5-dioxa-4-bora-tricyclo[6.1.1.0^{2,6}]dec-4-yl)-2-(trimethylsilanyl-amino)-ethyl]-benzoic acid tert-butyl ester solution dropwise at −20° C. The cooling bath was removed and the reaction stirred at room temperature. After 2 h the reaction was quenched with H₂O (100 mL) and the aqueous phase was extracted with EtOAc (3×100 mL), the combined organic layers were dried over MgSO₄, and concentrated in vacuo. The crude product was purified by flash column chromatography [R_{f}=0.24, silica gel (EtOAc/Hexane, 70:30, v/v)] to give a 47% yield of product. ESI-MS m/z 654 (MH)⁺.

Step 3. Synthesis of (1R)-(3-{[(pyridin-3-ylmethyl)-amino]-methyl}-benzoyl amino)-(3-carboxy-2-hydroxy)benzyl-methylboronic acid formate. Prepared from the BCl₃ reaction of 2-methoxy-3-[2-(3-{[(pyridin-3-ylmethyl)-amino]-methyl}-benzoylamino)-2-(2,9,9-trimethyl-3,5-dioxa-4-bora-tricyclo [6.1.1.02^{2,6}]dec-4-yl)-ethyl]-benzoic acid tert-butyl ester in DCM following the procedure described in Step 3 of Example 18. The crude product was purified by preparative HPLC using H₂O and MeOH solvents buffered with 0.1% formic acid to afford 30% of the product as a white solid. ESI-MS m/z 432 (MH−H₂O)⁺.

### Example 20

### (1R)-(3-piperazin-1-ylmethyl-benzoylamino-(3-carboxy-2-hydroxy)benzyl-methylboronic acid formate

Step 1. Synthesis of 3-[2-[3-(4-Boc-piperazin-1-ylmethyl)-benzoylamino]-2-(2,9,9-trimethyl-3,5-dioxa-4-bora-tricyclo[6.1.1.0^{2,6}]dec-4-yl)-ethyl]-benzoic acid tert-butyl ester. Prepared from the reductive amination of 3-[2-(3-formyl-benzoylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester with N-boc-piperazine following the procedure described in Step 2 of Example 18. The crude product was taken to next step without further purification. ESI-MS m/z 732 (MH)⁺.

Step 2. Synthesis of (1R)-(3-piperazin-1-ylmethyl-benzoylamino)-(3-carboxy-2-hydroxy)benzyl-methylboronic acid formate. Prepared from the BCl₃ reaction of 3-[2-[3-(4-Boc-piperazin-1-ylmethyl)-benzoylamino]-2-(2,9,9-trimethyl-3,5-dioxa-4-bora-tricyclo[6.1.1.0^{2,6}]dec-4-yl)-ethyl]-benzoic acid tert-butyl ester in DCM following the procedure described in Step 3 of Example 18. The crude product was purified by preparative HPLC using H₂O and MeOH solvents buffered with 0.1% formic acid to afford 54 mg (16%) of the product over two steps as a white solid. ESI-MS m/z 410 (MH−H₂O)⁺.

### Example 21

### (1R)-(3-methylaminomethyl-benzoylamino)-(3-carboxy-2-hydroxy)benzyl-methylboronic acid formate

Step 1. Synthesis of 2-methoxy-3-[2-(3-methylaminomethyl-benzoylamino)-2-(2,9,9-trimethyl-3,5-dioxa-4-bora-tricyclo[6.1.1.0^{2,6}]dec-4-yl)-ethyl]-benzoic acid tert-butyl ester. Prepared from the reductive amination of 3-[2-(3-Formyl-benzoylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester with methyl amine (2M solution in THF) following the procedure described in Step 2 of Example 18. The crude product was taken to next step without further purification. ESI-MS m/z 577 (MH)⁺.

Step 2. Synthesis of (1R)-(3-methylaminomethyl-benzoylamino)-(3-carboxy-2-hydroxy)benzyl-methylboronic acid formate. Prepared from the BCl₃ reaction of 2-methoxy-3-[2-(3-methylaminomethyl-benzoylamino)-2-(2,9,9-trimethyl-3,5-dioxa-4-bora-tricyclo[6.1.1.0^{2,6}]dec-4-yl)-ethyl]-benzoic acid tert-butyl ester in DCM following the procedure described in Step 3 of Example 18. The crude product was purified by preparative HPLC using H₂O and MeOH solvents buffered with 0.1% formic acid to afford 29 mg (10%) of the product over two steps as a white solid. ESI-MS m/z 355 (MH−H₂O)⁺.

### Example 22

### (1R)-[4-(1-amino-cyclopropyl)]-benzoylamino]-(3-carboxy-2-hydroxy)benzyl-methylboronic acid formate

Step 1. Synthesis of 4-(1-amino-cyclopropyl)-benzoic acid methyl ester. To 4-cyano-benzoic acid methyl ester (12 g, 74.6 mmol) in diethyl ether (300 ml) at −78° C. under argon titanium isopropoxide (23.3 g, 81.9 mmol) was added followed by slow addition of ethyl magnesium bromide (52.1 ml, 156.3 mmol, 3M solution in THF). After stirring the reaction mixture for 15 minutes, the cooling bath was removed. After stirring the mixture for 1 hour, BF₃.Et₂O was added slowly and mixture stirred for another 1 hour. The reaction was quenched with 1N HCl (75 ml) and extracted with Et₂O (3×100 ml). The aqueous layer was basified with 3N NaOH solution and extracted with Et₂O (3×100 ml). The ether layer was combined and was dried over MgSO₄, and concentrated in vacuo. The crude product which was obtained in 41% was taken to next step without purification. ESI-MS m/z 192 (MH)⁺.

Step 2. Synthesis of 4-(1-tert-butoxycarbonylamino-cyclopropyl)-benzoic acid. The crude 4-(1-amino-cyclopropyl)-benzoic acid methyl ester (4.0 g, 20.9 mmol) which was made in the previous step was dissolved in CH₃CN (80 ml), boc₂O (4.8 g, 21.9) was then added and the solution stirred at room temperature for 6 hours. The reaction was quenched with water (100 ml) and extracted with EtOAc (3×100 ml). The organic layers were combined and were dried over MgSO₄, and concentrated in vacuo to obtained crude 4-(1-tert-butoxycarbonylamino-cyclopropyl)-benzoic acid methyl ester. ESI-MS m/z 292 (MH)⁺. To this crude product in H₂O (60 ml) and MeOH (150 ml), NaOH (2.4 g, 60 mmol) was added and the mixture stirred 15 hours at room temperature. MeOH was evaporated, and more water (200 ml) was added. 1N HCl was added slowly which caused the precipitation of the product. The solid was filtered off to give 1.97 g of pure 4-(1-tert-butoxycarbonylamino-cyclopropyl)-benzoic acid in 72% yield. ESI-MS m/z 278 (MH)⁺.

Step 3. Synthesis of 3-[2-[4-(1-tert-butoxycarbonylamino-cyclopropyl)-benzoylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester. Prepared from the 2-methoxy-3-(2,9,9-trimethyl-3,5-dioxa-4-bora-tricyclo[6.1.1.0^{2,6}]dec-4-ylmethyl)-benzoic acid tert-butyl ester and 4-(1-tert-butoxycarbonylamino-cyclopropyl)-benzoic acid following the procedure described in Step 2 of Example 18. The crude product was purified by flash column chromatography [R_{f}=0.24, silica gel (EtOAc/Hexane, 40:60, v/v)] to give the coupled product in 31% yield. ESI-MS m/z 689 (MH)⁺.

Step 4. Synthesis of (1R)-(4-(1-amino-cyclopropyl)]-benzoylamino]-(3-carboxy-2-hydroxy)benzyl-methylboronic acid formate. Prepared from the BCl₃ (7.2 ml, 7.2 mmol, 1M solution in DCM) reaction of 3-[2-[4-(1-tert-butoxycarbonylamino-cyclopropyl)-benzoylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester (0.72 mmol) in DCM following the procedure described in Step 3 of Example 18. The crude product was purified by preparative HPLC using H₂O and MeOH solvents buffered with 0.1% formic acid to afford 97 mg (42%) of the product over two steps as a white solid. ESI-MS m/z 367 (MH−H₂O)⁺.

### Example 23

### (1R)-(3-{[bis-(2-hydroxy-ethyl)-amino]-methyl}-benzoylamino)-(3-carboxy-2-hydroxy)benzyl-methylboronic acid formate

Step 1. Synthesis of 3-[2-(3-{[bis-(2-hydroxy-ethyl)-amino]-methyl}-benzoylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester. Prepared from the reductive amination of 3-[2-(3-formyl-benzoylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester with diethanolamine following the procedure described in Step 2 of Example 18. The crude product was taken to next step without further purification. ESI-MS m/z 651 (MH)⁺.

Step 2. Synthesis of (1R)-(3-{[bis-(2-hydroxy-ethyl)-amino]-methyl}-benzoylamino)-(3-carboxy-2-hydroxy)benzyl-methylboronic acid formate. Prepared from the BCl₃ reaction of 3-[2-(3-{[bis-(2-hydroxy-ethyl)-amino]-methyl}-benzoylamino)-2-(2,9,9-trimethyl-3,5-dioxa-4-bora-tricyclo[6.1.1.02,6]dec-4-yl)-ethyl]-2-methoxy-benzoic acid tert-butyl ester in DCM following the procedure described in Step 3 of Example 18. The crude product was purified by preparative HPLC using H₂O and MeOH solvents buffered with 0.1% formic acid to afford 38 mg (13%) of the product over two steps as a white solid. ESI-MS m/z 429 (MH−H₂O)⁺.

### Example 24

### (1R)-(3-{[acetyl-(2-amino-ethyl)-amino]-methyl}-benzoylamino)-(3-carboxy-2-hydroxy)benzyl-methylboronic acid formate

Step 1. Synthesis of 3-[2-(3-{[acetyl-(2-tert-butoxycarbonylamino-ethyl)-amino]-methyl}-benzoylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester. To a solution of 3-[2-{3-[(2-tert-butoxycarbonylamino-ethylamino)-methyl]-benzoylamino}-2-(2,9,9-trimethyl-3,5-dioxa-4-bora tricyclo[6.1.1.0^{2,6}]dec-4-yl)-ethyl]-2-methoxy-benzoic acid tert-butyl ester (400 mg, 0.71 mmol), prepared as described in Example 18, in methylene chloride (7 ml), at 0° C. under argon was added pyridine (84 mg, 1.06 mmol) followed by acetic anhydride (109 mg, 0.92 mmol). The ice bath was removed and the mixture stirred at room temperature. After 4 hours LCMS indicated the completion of the reaction. H₂O (30 mL) was added to quench the reaction and the aqueous phase was extracted with DCM (3×50 mL), the combined organic layers were washed with saturated NaHCO₃ (10 ml) followed by 1M HCl (10 ml) and then dried over MgSO₄, and concentrated in vacuo. The crude product was taken to the next step without purification. ESI-MS m/z 748 (MH)⁺.

Step 2. Synthesis of (1R)-(3-{[acetyl-(2-amino-ethyl)-amino]-methyl}-benzoylamino)-(3-carboxy-2-hydroxy)benzyl-methylboronic acid formate. Prepared from the BCl₃ reaction of 3-[2-(3-{[acetyl-(2-tert-butoxycarbonyl amino-ethyl)-amino]-methyl]-benzoylamino)-2-(2,9,9-trimethyl-3,5-dioxa-4-bora-tricyclo [6.1.1.0^{2,6}]dec-4-yl)-ethyl]-2-methoxy-benzoic acid tert-butyl ester in DCM following the procedure described in Step 3 of Example 18. The crude product was purified by preparative HPLC using H₂O and MeOH solvents buffered with 0.1% formic acid to afford 30% of the product over three steps as a white solid. ESI-MS m/z 426 (MH−H₂O)⁺.

### Example 25

### (1R)-[3-{(3-hydroxy-propylamino)-methyl}-benzoylamino]-(3-carboxy-2hydroxy)benzyl-methylboronic acid formate

Step 1. Synthesis of 3-[2-{3-[(3-hydroxy-propylamino)-methyl]-benzoylamino}-2-(2,9,9-trimethyl-3,5-dioxa-4-bora-tricyclo[6.1.1.02,6]dec-4-yl)-ethyl]-2-methoxy-benzoic acid tert-butyl ester. Prepared from the reductive amination of 3-[2-(3-formyl-benzoylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester with propanolamine following the procedure described in Step 2 of Example 18. The crude product was taken to next step without further purification. ESI-MS m/z 621 (MH)⁺.

Step 2. Synthesis of (1R)-[3-(3-hydroxy-propylamino)-methyl]-benzoylamino-(3-carboxy-2-hydroxy)benzyl-methylboronic acid formate. Prepared from the BCl₃ reaction of 3-[2-{3-[(3-hydroxy-propylamino)-methyl]-benzoylamino}-2-(2,9,9-trimethyl-3,5-dioxa-4-bora-tricyclo[6.1.1.02,6]dec-4-yl)-ethyl]-2-methoxy-benzoic acid tert-butyl ester in DCM following the procedure described in Step 3 of Example 18. The crude product was purified by preparative HPLC using H₂O and MeOH solvents buffered with 0.1% formic acid to afford 30% of the product as a white solid. ESI-MS m/z 399 (MH−H₂O)⁺.

### Example 26

### (1R)-[3-(pyridin-3-ylaminomethyl)-benzoylamino]-(3-carboxy-2hydroxy)benzyl-methylboronic acid formate

Step 1. Synthesis of 2-methoxy-3-(2-[3-(pyridin-3-ylaminomethyl)-benzoylamino]-2-(2,9,9-trimethyl-3,5-dioxa-4-bora-tricyclo[6.1.1.0^{2,6}]dec-4-yl)-ethyl]-benzoic acid tert-butyl ester. Prepared from the reductive amination of 3-[2-(3-formyl-benzoylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester with 3-amino-pyridine following the procedure described in Step 2 of Example 18. The crude product, which was a 2:1 ratio of desired product to reduced alcohol, was taken to next step without further purification. ESI-MS m/z 640 (MH)⁺.

Step 2. (1R)-[3-(pyridin-3-ylaminomethyl)-benzoylamino]-(3-carboxy-2hydroxy)benzyl-methylboronic acid formate. Prepared from the BCl₃ reaction of 2-methoxy-3-[2-[3-(pyridin-3-ylaminomethyl)-benzoylamino]-2-(2,9,9-trimethyl-3,5-dioxa-4-bora-tricyclo[6.1.1.0^{2,6}]dec-4-yl)-ethyl]-benzoic acid tert-butyl ester in DCM following the procedure described in Step 3 of Example 18. The crude product was purified by preparative HPLC using H₂O and MeOH solvents buffered with 0.1% formic acid to afford 20% the product as a white solid over two steps. ESI-MS m/z 418 (MH−H₂O)⁺.

### Example 27

### (1R)-(3-dimethylaminomethyl-benzoylamino)-(3-carboxy-2-hydroxy)benzyl-methylboronic acid formate

Step 1. Synthesis of 2-methoxy-3-[2-(3-dimethylaminomethyl-benzoylamino)-2-(2,9,9-trimethyl-3,5-dioxa-4-bora-tricyclo[6.1.1.0^{2,6}]dec-4-yl)-ethyl]-benzoic acid tert-butyl ester. Prepared from the reductive amination of 3-[2-(3-formyl-benzoylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester with dimethylamine (2M solution in THF) following the procedure described in Step 2 of Example 18. The crude product was taken to next step without further purification. ESI-MS m/z 591 (MH)⁺.

Step 2. Synthesis of (1R)-(3-dimethylaminomethyl-benzoylamino)-(3-carboxy-2-hydroxy)benzyl-methylboronic acid formate. Prepared from the BCl₃ reaction of 2-methoxy-3-[2-(3-dimethylaminomethyl-benzoylamino)-2-(2,9,9-trimethyl-3,5-dioxa-4-bora-tricyclo[6.1.1.0^{2,6}]dec-4-yl)-ethyl]-benzoic acid tert-butyl ester in DCM following the procedure described in Step 3 of Example 18. The crude product was purified by preparative HPLC using H₂O and MeOH solvents buffered with 0.1% formic acid to afford 32% of the product over two steps as a white solid. ESI-MS m/z 369 (MH−H₂O)⁺.

### Example 28

### (1R)-[3-{(5-methyl-isoxazol-3-ylamino)-methyl}-benzoylamino]-(3-carboxy-2hydroxy)benzyl-methylboronic acid formate

Step 1. Synthesis of 2-methoxy-3-[2-{3-[(5-methyl-isoxazol-3-ylamino)-methyl]-benzoylamino}-2-(2,9,9-trimethyl-3,5-dioxa-4-bora-tricyclo [6.1.1.0^{2,6}]dec-4-yl)-ethyl]benzoic acid tert-butyl ester. Prepared from the reductive amination of 3-[2-(3-formyl-benzoylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester with 5-methyl-isoxazol-3-ylamine following the procedure described in Step 2 of Example 18. The crude product, which was a 1:1 ratio of the desired product to unwanted reduced alcohol, was taken to next step without further purification. ESI-MS m/z 644 (MH)⁺.

Step 2. Synthesis of (1R)-[3-{(5-methyl-isoxazol-3-ylamino)-methyl}-benzoylamino]-(3-carboxy-2hydroxy)benzyl-methylboronic acid formate. Prepared from the BCl₃ reaction of 2-methoxy-3-[2-{3-[(5-methyl-isoxazol-3-ylamino)-methyl]-benzoylamino}-2-(2,9,9-trimethyl-3,5-dioxa-4-bora-tricyclo [6.1.1.0^{2,6}]dec-4-yl)-ethyl]-benzoic acid tert-butyl ester in DCM following the procedure described in Step 3 of Example 18. The crude product was purified by preparative HPLC using H₂O and MeOH solvents buffered with 0.1% formic acid to afford 12% of the product over two steps as a white solid. ESI-MS m/z 422 (MH−H₂O)⁺.

### Example 29

### (1R)-[2-(3-aminomethyl-phenyl)-acetylamino]-(3-carboxy-2hydroxy)benzyl-methylboronic acid formate

Step 1. Synthesis of 3-[2-{2-[3-(tert-butoxycarbonylamino-methyl)-phenyl]-acetylamino}-2-(2,9,9-trimethyl-3,5-dioxa-4-bora-tricyclo[6.1.1.0^{2,6}]dec-4-yl)-ethyl]-2-methoxy-benzoic acid tert-butyl ester. Prepared from the 2-methoxy-3-(2,9,9-trimethyl-3,5-dioxa-4-bora-tricyclo[6.1.1.0^{2,6}]dec-4-ylmethyl)-benzoic acid tert-butyl ester and [3-(tert-butoxycarbonylamino-methyl)-phenyl]-acetic acid following the procedure described in Step 2 of Example 19. The crude product was purified by flash column chromatography [R_{f}=0.22, silica gel (EtOAc/Hexane, 40:60, v/v)] to give the coupled product in 41% yield. ESI-MS m/z 677 (MH)⁺.

Step 2. Synthesis of (1R)-(2-(3-aminomethyl-phenyl)-acetylamino]-(3-carboxy-2hydroxy)benzyl-methylboronic acid formate. Prepared from the BCl₃ reaction of 3-[2-{2-[3-(tert-butoxycarbonylamino-methyl)-phenyl]-acetylamino}-2-(2,9,9-trimethyl-3,5-dioxa-4-bora-tricyclo[6.1.1.0^{2,6}]dec-4-yl)-ethyl]-2-methoxy-benzoic acid tert-butyl ester in DCM following the procedure described in Step 3 of Example 18. The crude product was purified by preparative HPLC using H₂O and MeOH solvents buffered with 0.1% formic acid to afford 47% of the product as a white solid. ESI-MS m/z 355 (MH−H₂O)⁺.

### Example 30

### (1R)-[2-(4-aminomethyl-phenyl)-acetylamino]-(3-carboxy-2hydroxy)benzyl-methylboronic acid formate

Step 1. Synthesis of 3-[2-{2-[4-(tert-butoxycarbonylamino-methyl)-phenyl]-acetylamino}-2-(2,9,9-trimethyl-3,5-dioxa-4-bora-tricyclo[6.1.1.0^{2,6}]dec-4-yl)-ethyl]-2-methoxy-benzoic acid tert-butyl ester. Prepared from the 2-methoxy-3-(2,9,9-trimethyl-3,5-dioxa-4-bora-tricyclo[6.1.1.0^{2,6}]dec-4-ylmethyl)-benzoic acid tert-butyl ester and 4-bocaminomethyl-phenylacetic acid following the procedure described in Step 2 of Example 19. The crude product was purified by flash column chromatography [R_{f}=0.22, silica gel (EtOAc/Hexane, 40:60, v/v)] to give the coupled product in 40% yield. ESI-MS m/z 677 (MH)⁺.

Step 2. Synthesis of (1R)-1-[2-(4-aminomethyl-phenyl)-acetylamino]-1-(3-carboxy-2hydroxy)benzyl-methylboronic acid formate. Prepared from the BCl₃ reaction of 3-[2-{2-[4-(tert-butoxycarbonylamino-methyl)-phenyl]-acetylamino}-2-(2,9,9-trimethyl-3,5-dioxa-4-bora-tricyclo[6.1.1.0^{2,6}]dec-4-yl)-ethyl]-2-methoxy-benzoic acid tert-butyl ester in DCM following the procedure described in Step 3 of Example 18. The crude product was purified by preparative HPLC using H₂O and MeOH solvents buffered with 0.1% formic acid to afford 52% of the product as a white solid. ESI-MS m/z 355 (MH−H₂O)⁺.

### Example 31

### (1R)-[3-(thiazol-2-ylaminomethyl)-benzoylamino]-(3-carboxy-2-hydroxy)benzyl-methylboronic acid

Step 1. Synthesis of 2-methoxy-3-[2-[3-(thiazol-2-ylaminomethyl)-benzoylamino]-2-(2,9,9-trimethyl-3,5-dioxa-4-bora-tricyclo[6.1.1.0^{2,6}]dec-4-yl)-ethyl]-benzoic acid tert-butyl ester. To a solution of 3-[2-(3-formyl-benzoylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester (400 mg, 0.71 mmol) in MeOH (5 ml) was added 2-aminothiazole (74 mg, 0.74 mmol) followed by AcOH (56 mg, 0.92 mmol). The mixture was stirred for 24 hrs, followed by sonication for 1.5 hours at room temperature. LCMS indicated the formation of at least 40% of the imine. NaBH₄ (53 mg, 1.42 mmol) was then added and the mixture stirred for 1 hour. Solvent was removed under vacuum, water (20 ml) was added and the residues were extracted with EtOAc (3×50 mL), the combined organic layers were washed with saturated NaHCO₃ (10 ml) followed by brine (15 ml), then dried over MgSO₄, and concentrated in vacuo. The crude product was purified by flash column chromatography [R_{f}=0.20 silica gel (EtOAc/Hexane, 60:40, v/v)] to give 180 mg of the desired product in 28% yield. ESI-MS m/z 646 (MH)⁺.

Step 2. Synthesis of (1R)-[3-(thiazol-2-ylaminomethyl)-benzoylamino]-(3-carboxy-2-hydroxy)benzyl-methylboronic acid. Prepared from the BCl₃ reaction of 2-methoxy-3-[2-[3-(thiazol-2-ylaminomethyl)-benzoylamino]-2-(2,9,9-trimethyl-3,5-dioxa-4-bora-tricyclo[6.1.1.0^{2,6}]dec-4-yl)-ethyl]-benzoic acid tert-butyl ester in DCM following the procedure described in Step 3 of Example 18. The crude product was purified by preparative HPLC using H₂O and MeOH solvents buffered with 0.1% formic acid to afford 19% of the product as a white solid. ESI-MS m/z 424 (MH−H₂O)⁺.

### Example 32

### (1R)-[2-(4-acetylaminomethyl-phenyl)-acetylamino]-(3-carboxy-2hydroxy)benzyl-methylboronic acid

Step 1. Synthesis of 3-[2-{2-[4-(acetylamino-methyl)-phenyl]-acetylamino}-2-(2,9,9-trimethyl-3,5-dioxa-4-bora-tricyclo[6.1.1.0^{2,6}]dec-4-yl)-ethyl]-2-methoxy-benzoic acid. To a solution of 3-[2-{2-[4-(tert-butoxycarbonylamino-methyl)-phenyl]-acetylamino}-2-(2,9,9-trimethyl-3,5-dioxa-4-bora-tricyclo[6.1.1.0^{2,6}]dec-4-yl)-ethyl]-2-methoxy-benzoic acid tert-butyl ester (620 mg, 0.92 mmol) in DCM (10 ml) was added trifluoroacetic acid (TFA) (0.5 ml, 6.42 mmol) and the mixture stirred at room temperature. After 2 hours LCMS indicated the consumption of all of the starting material. Solvent was evaporated under vacuum. The residue of 3-[2-[2-(4-aminomethyl-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid TFA salt so obtained was dissolved in DCM (10 ml). Acetic anhydride (0.17 ml, 1.84 mmol) and pyridine (0.3 ml, 3.68 mmol) were added at 0° C. under argon. The mixture stirred at room temperature overnight. Water (20 ml) was added and the residues were extracted with DCM (3×50 mL), the combined organic layers were washed with saturated 1N HCl (10 ml) followed by brine (15 ml) and then dried over MgSO₄, and concentrated in vacuo. The crude product was taken to next step without further purification. ESI-MS m/z 563 (MH)⁺.

Step 2. Synthesis of (1R)-(2-(4-acetylaminomethyl-phenyl)-acetylamino]-(3-carboxy-2hydroxy)benzyl-methylboronic acid. Prepared from the BCl₃ reaction of 3-[2-{2-[4-(acetylamino-methyl)-phenyl]-acetylamino}-2-(2,9,9-trimethyl-3,5-dioxa-4-bora-tricyclo[6.1.1.0^{2,6}]dec-4-yl)-ethyl]-2-methoxy-benzoic acid in DCM following the procedure described in Step 3 of Example 18. The crude product was purified by preparative HPLC using H₂O and MeOH solvents buffered with 0.1% formic acid to afford 18% of the product as a white solid. ESI-MS m/z 397 (MH−H₂O)⁺.

### Example 33

### (1R)-[2-(4-methylaminomethyl-phenyl)-acetylamino]-(3-carboxy-2hydroxy)benzyl-methylboronic acid formate

Step 1. Synthesis of 3-[2-(2-(4-bromomethyl-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester. To anhydrous CH₂Cl₂ (2.08 mL, 32.5 mmol) in anhydrous THF (80 mL) under argon at −100° C. [MeOH, liq. N₂ slush bath], n-BuLi (12 mL, 2.5M in hexane, 30 mmol) was added dropwise and the mixture was stirred for 30 minutes. A THF (15 mL) solution of 2-methoxy-3-(2,9,9-trimethyl-3,5-dioxa-4-bora-tricyclo[6.1.1.0^{2,6}]dec-4-ylmethyl)-benzoic acid tert-butyl ester (10.0 g, 25 mmol) was added over a period of 20 minutes. After 40 minutes the cooling bath was removed and the mixture warmed slowly to 0° C. After 1 hour the reaction flask was cooled to −78° C., LHMDS (27.5 mL, 1M in THF, 27.5 mmol) was added slowly and the resultant solution was warmed to room temperature gradually while stirring overnight. Anhydrous MeOH (1.11 mL, 27.5 mmol) was added at −10° C., the reaction stirred for 1 h at the same temperature and then for 1 h at room temperature. At this stage LCMS indicated the formation of 2-methoxy-3-[2-(2,9,9-trimethyl-3,5-dioxa-4-bora-tricyclo[6.1.1.0^{2,6}]dec-4-yl)-2-(trimethylsilanyl-amino)-ethyl]-benzoic acid tert-butyl ester intermediate.

To a separate dry round bottom flask under argon containing DMF 2.31 ml, 30 mmol) in Et₂O (65 ml) at 0° C., oxalyl chloride (2.61 ml, 30.0 mmol) was added with constant stirring. Gas evolution ceased in ca. 5 minutes, and a colourless precipitate formed. Ether was evaporated under vacuum, and to solid salt so obtained was added DCM (40 ml). The suspension was cooled to −20° C. and 4-bromomethyl-phenylacetic acid (6.87 g, 30 mmol) was added at once. In less than 5 minutes, all of the material went in solution, indicating that 4-bromomethyl-phenylacetyl chloride has formed. To the previously prepared solution of 2-methoxy-3-[2-(2,9,9-trimethyl-3,5-dioxa-4-bora-tricyclo[6.1.1.0^{2,6}]dec-4-yl)-2-(trimethylsilanyl-amino)-ethyl]-benzoic acid tert-butyl ester intermediate at −78° C. was added pyridine (2.42 ml, 30 mmol) followed by freshly prepared solution of 4-bromomethyl-phenylacetyl chloride. After stirring at 45 minutes at the same temperature cooling bath was removed and the reaction stirred at room temperature. After 2 h the reaction was quenched with H₂O (150 mL) and the aqueous phase was extracted with EtOAc (3×100 mL), the combined organic layers were washed with saturated NaHCO₃ (40 ml) followed by brine (50 ml) and then dried over MgSO₄, and concentrated in vacuo. The crude product was purified by flash column chromatography [R_{f}=0.35, silica gel (EtOAc/Hexane, 40:60, v/v)] to give 7.2 g of the coupled product in 45% yield. ESI-MS m/z 640 (MH)⁺

Step 2. Synthesis of 2-methoxy-3-[2-[2-(4-methylaminomethyl-phenyl)-acetylamino]-2-(2,9,9-trimethyl-3,5-dioxa-4-bora-tricyclo[6.1.1.0^{2,6}]dec-4-yl)-ethyl]-benzoic acid tert-butyl ester. To 3-[2-[2-(4-bromomethyl-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester (412 mg, 0.69 mmol) in DCM (6 ml) was added tetrabutylammonium iodide (184 mg, 0.34 mmol) and methylamine (0.8 ml, 1.6 mmol, 2 M solution in THF). The mixture was stirred for 18 hours at room temperature. The reaction was quenched with H₂O (30 mL) and the aqueous phase was extracted with DCM (3×30 mL), the combined organic layers were washed with brine (15 ml) and then dried over MgSO₄, and concentrated in vacuo. The crude product was taken to next step without further purification. ESI-MS m/z 591 (MH)⁺

Step 3. Synthesis of (1R)-(2-(4-methylaminomethyl-phenyl)-acetylamino]-(3-carboxy-2hydroxy)benzyl-methylboronic acid formate. Prepared from the BCl₃ reaction of 2-methoxy-3-[2-[2-(4-methylaminomethyl-phenyl)-acetylamino]-2-(2,9,9-trimethyl-3,5-dioxa-4-bora-tricyclo[6.1.1.0^{2,6}]dec-4-yl)-ethyl]-benzoic acid tert-butyl ester in DCM following the procedure described in Step 3 of Example 18. The crude product was purified by preparative HPLC using H₂O and MeOH solvents buffered with 0.1% formic acid to afford 10% of the product over two steps as a white solid. ESI-MS m/z 369 (MH−H₂O)⁺.

### Example 34

### (1R)-[2-{4-((2-hydroxy-ethylamino)-methyl)-phenyl}-acetylamino]-(3-carboxy-2hydroxy)benzyl-methylboronic acid formate

Step 1. Synthesis of 3-[2-(2-{4-[(2-hydroxy-ethylamino)-methyl]-phenyl}-acetylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester.

Prepared from the 3-[2-[2-(4-bromomethyl-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester with ethanolamine following the procedure described in Step 2 of Example 23. The crude product was taken to next step without further purification. ESI-MS m/z 622 (MH)⁺.

Step 2. Synthesis of (1R)-(2-{4-((2-hydroxy-ethylamino)-methyl)-phenyl}-acetylamino]-(3-carboxy-2hydroxy)benzyl-methylboronic acid formate. Prepared from the BCl₃ reaction of 3-[2-(2-{4-[(2-hydroxy-ethylamino)-methyl]-phenyl}-acetylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester in DCM following the procedure described in Step 3 of Example 18. The crude product was purified by preparative HPLC using H₂O and MeOH solvents buffered with 0.1% formic acid to afford 10% of the product over two steps as a white solid. ESI-MS m/z 399 (MH−H₂O)⁺.

### Example 35

### (1R)-[2-{4-((2-amino-ethylamino)-methyl)-phenyl}-acetylamino]-(3-carboxy-2hydroxy)benzyl-methylboronic acid formate

Step 1. Synthesis of 3-[2-(2-{4-[(2-tert-butoxycarbonylamino-ethylamino)-methyl]-phenyl}-acetylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester. Prepared from the 3-[2-[2-(4-bromomethyl-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester and N-Boc-ethylenediamine following the procedure described in Step 2 of Example 33. The crude product was purified by flash column chromatography [R_{f}=0.23 silica gel (MeOH/DCM, 05:95, v/v)] to give 290 mg of the desired product in 45% yield. ESI-MS m/z 720 (MH)⁺.

Step 2. Synthesis of (1R)-(2-{4-((2-amino-ethylamino)-methyl)-phenyl}-acetylamino]-(3-carboxy-2hydroxy)benzyl-methylboronic acid formate. Prepared from the BCl₃ reaction of 3-[2-(2-{4-[(2-tert-butoxycarbonylamino-ethylamino)-methyl]-phenyl}-acetylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester in DCM following the procedure described in Step 3 of Example 18. The crude product was purified by preparative HPLC using H₂O and MeOH solvents buffered with 0.1% formic acid to afford 22% of the product over two steps as a white solid. ESI-MS m/z 399 (MH−H₂O)⁺.

### Example 36

### (1R)-[2-(4-dimethylaminomethyl-phenyl)-acetylamino]-(3-carboxy-2hydroxy)benzyl-methylboronic acid formate

Step 1. Synthesis of 2-methoxy-3-[2-[2-(4-dimethylaminomethyl-phenyl)-acetylamino]-2-(2,9,9-trimethyl-3,5-dioxa-4-bora-tricyclo[6.1.1.0^{2,6}]dec-4-yl)-ethyl]-benzoic acid tert-butyl ester. Prepared from the 3-[2-[2-(4-bromomethyl-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester and dimethyl amine (2M solution in THF) following the procedure described in Step 2 of Example 33. The crude product was taken to next step without further purification. ESI-MS m/z 605 (MH)⁺

Step 2. Synthesis of (1R)-(2-(4-dimethylaminomethyl-phenyl)-acetylamino]-(3-carboxy-2hydroxy)benzyl-methylboronic acid formate. Prepared from the BCl₃ reaction of 2-methoxy-3-[2-[2-(4-dimethylaminomethyl-phenyl)-acetylamino]-2-(2,9,9-trimethyl-3,5-dioxa-4-bora-tricyclo[6.1.1.0^{2,6}]dec-4-yl)-ethyl]-benzoic acid tert-butyl ester in DCM following the procedure described in Step 3 of Example 18. The crude product was purified by preparative HPLC using H₂O and MeOH solvents buffered with 0.1% formic acid to afford 18% of the product over two steps as a white solid. ESI-MS m/z 383 (MH−H₂O)⁺.

### Example 37

### (1R)-[2-(4-piperazin-1-ylmethyl-phenyl)-acetylamino]-(3-carboxy-2hydroxy)benzyl-methylboronic acid formate

Step 1. Synthesis of 4-(4-{[2-(3-tert-butoxycarbonyl-2-methoxy-phenyl)-1-(2,9,9-trimethyl-3,5-dioxa-4-bora-tricyclo[6.1.1.0^{2,6}]dec-4-yl)-ethylcarbamoyl]-methyl}-benzyl)-piperazine-1-carboxylic acid tert-butyl ester. Prepared from the 3-[2-[2-(4-bromomethyl-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester and piperazine-1-carboxylic acid tert-butyl ester following the procedure described in Step 2 of Example 33. The crude product was taken to next step without further purification. ESI-MS m/z 746 (MH)⁺

Step 2. Synthesis of (1R)-(2-(4-piperazin-1-ylmethyl-phenyl)-acetylamino]-(3-carboxy-2hydroxy)benzyl-methylboronic acid formate. Prepared from the BCl₃ reaction of 4-(4-{[2-(3-tert-butoxycarbonyl-2-methoxy-phenyl)-1-(2,9,9-trimethyl-3,5-dioxa-4-bora-tricyclo[6.1.1.0^{2,6}]dec-4-yl)-ethylcarbamoyl]-methyl}-benzyl)-piperazine-1-carboxylic acid tert-butyl ester in DCM following the procedure described in Step 3 of Example 18. The crude product was purified by preparative HPLC using H₂O and MeOH solvents buffered with 0.1% formic acid to afford 29% of the product over two steps as a white solid. ESI-MS m/z 424 (MH−H₂O)⁺.

### Example 38

### (1R)-[2-(4-pyrrolin-1-ylmethyl-phenyl)-acetylamino]-(3-carboxy-2-hydroxy)benzyl-methylboronic acid formate

Step 1. Synthesis of 2-methoxy-3-[2-[2-(4-pyrrolidin-1-ylmethyl-phenyl)-acetylamino]-2-(2,9,9-trimethyl-3,5-dioxa-4-bora-tricyclo[6.1.1.0^{2,6}]dec-4-yl)-ethyl]-benzoic acid tert-butyl ester. Prepared from the 3-[2-[2-(4-bromomethyl-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester and pyrrolidine following the procedure described in Step 2 of Example 33. The crude product was taken to next step without further purification. ESI-MS m/z 631 (MH)⁺

Step 2. Synthesis of (1R)-(2-(4-pyrrolin-1-ylmethyl-phenyl)-acetylamino]-(3-carboxy-2hydroxy)benzyl-methylboronic acid formate. Prepared from the BCl₃ reaction of 2-methoxy-3-[2-[2-(4-pyrrolidin-1-ylmethyl-phenyl)-acetylamino]-2-(2,9,9-trimethyl-3,5-dioxa-4-bora-tricyclo[6.1.1.0^{2,6}] dec-4-yl)-ethyl]-benzoic acid tert-butyl ester in DCM following the procedure described in Step 3 of Example 18. The crude product was purified by preparative HPLC using H₂O and MeOH solvents buffered with 0.1% formic acid to afford 31% of the product over two steps as a white solid. ESI-MS m/z 409 (MH−H₂O)⁺.

### Example 39

### (1R)-[2-(4-{[bis-(2-hydroxy-ethyl)-amino]-methyl}-phenyl)-acetylamino]-(3-carboxy-2-hydroxy)benzyl-methylboronic acid formate

Step 1. Synthesis of 3-[2-[2-(4-{[bis-(2-hydroxy-ethyl)-amino]-methyl}-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester. Prepared from the 3-[2-[2-(4-bromomethyl-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester and diethanolamine following the procedure described in Step 2 of Example 33. The crude product was taken to next step without further purification. ESI-MS m/z 665 (MH)⁺.

Step 2. Synthesis of (1R)-[2-(4-{[bis-(2-hydroxy-ethyl)-amino]-methyl}-phenyl)-acetylamino]-(3-carboxy-2-hydroxy)benzyl-methylboronic acid formate. Prepared from the BCl₃ reaction of 3-[2-[2-(4-{[bis-(2-hydroxy-ethyl)-amino]-methyl}-phenyl)-acetylamino]-2-(2,9,9-trimethyl-3,5-dioxa-4-bora-tricyclo [6.1.1.0^{2,6}]dec-4-yl)-ethyl]-2-methoxy-benzoic acid tert-butyl ester in DCM following the procedure described in Step 3 of Example 18. The crude product was purified by preparative HPLC using H₂O and MeOH solvents buffered with 0.1% formic acid to afford 20% of the product over two steps as a white solid. ESI-MS m/z 443 (MH−H₂O)⁺.

### Example 40

### (1R)-2-amino-(4-{[2-(3-carboxy-2-hydroxy-phenyl-borono-ethylcarbamoyl]-methyl}-benzyl)-pyridinium formate

Step 1. Synthesis of 2-amino-1-(4-{[2-(3-tert-butoxycarbonyl-2-methoxy-phenyl)-1-(2,9,9-trimethyl-3,5-dioxa-4-bora-tricyclo[6.1.1.0^{2,6}]dec-4-yl)-ethylcarbamoyl]-methyl}-benzyl)-pyridinium chloride. Prepared from the 3-[2-[2-(4-bromomethyl-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester and 2-aminopyridine following the procedure described in Step 2 of Example 33. The crude product was taken to next step without further purification. ESI-MS m/z 655 (MH)⁺.

Step 2. Synthesis of (1R)-2-amino-(4-{[2-(3-carboxy-2-hydroxy-phenyl)-1-borono-ethylcarbamoyl]-methyl}-benzyl)-pyridinium formate. Prepared from the BCl₃ reaction of 2-amino-1-(4-{[2-(3-tert-butoxycarbonyl-2-methoxy-phenyl)-1-(2,9,9-trimethyl-3,5-dioxa-4-bora-tricyclo[6.1.1.0^{2,6}]dec-4-yl)-ethylcarbamoyl]-methyl}-benzyl)-pyridinium chloride in DCM following the procedure described in Step 3 of Example 18. The crude product was purified by preparative HPLC using H₂O and MeOH solvents buffered with 0.1% formic acid to afford 20% of the product over two steps as a white solid. ESI-MS m/z 433 (MH−H₂O)⁺.

### Example 41

### 4-(4-{[2-(3-carboxy-2-hydroxy-phenyl)-1-borono-ethylcarbamoyl]-methyl}-benzyl)-4-methyl-morpholin-4-ium formate

Step 1. Synthesis of 4-(4-{[2-(3-tert-butoxycarbonyl-2-methoxy-phenyl)-1-(2,9,9-trimethyl-3,5-dioxa-4-bora-tricyclo[6.1.1.0^{2,6}]dec-4-yl)-ethylcarbamoyl]-methyl}-benzyl)-4-methyl-morpholin-4-ium chloride. Prepared from the 3-[2-[2-(4-bromomethyl-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester and N-methyl morpholine following the procedure described in Step 2 of Example 33. The crude product was taken to next step without further purification. ESI-MS m/z 662 (MH)⁺.

Step 2. Synthesis of 4-(4-{[2-(3-carboxy-2-hydroxy-phenyl)-1-borono-ethyl carbamoyl]-methyl}-benzyl)-4-methyl-morpholin-4-ium formate. Prepared from the BCl₃ reaction of 4-(4-{[2-(3-tert-butoxycarbonyl-2-methoxy-phenyl)-1-(2,9,9-trimethyl-3,5-dioxa-4-bora-tricyclo[6.1.1.0^{2,6}]dec-4-yl)-ethylcarbamoyl]-methyl}-benzyl)-4-methyl-morpholin-4-ium chloride in DCM following the procedure described in Step 3 of Example 18. The crude product was purified by preparative HPLC using H₂O and MeOH solvents buffered with 0.1% formic acid to afford 32% of the product over two steps as a white solid. ESI-MS m/z 440 (MH−H₂O)⁺.

### Example 42

### 1-(4-{[2-(3-ethyl-2-hydroxy-phenyl)-1-sulfo-ethylcarbamoyl]-methyl}-benzyl)-pyridinium formate

Step 1. Synthesis of 1-(4-{[2-(3-tert-butoxycarbonyl-2-methoxy-phenyl)-1-(2,9,9-trimethyl-3,5-dioxa-4-bora-tricyclo[6.1.1.0^{2,6}]dec-4-yl)-ethylcarbamoyl]-methyl}-benzyl)-pyridinium chloride. Prepared from the 3-[2-[2-(4-bromomethyl-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester and pyridine following the procedure described in Step 2 of Example 33. The crude product was taken to next step without further purification. ESI-MS m/z 640 (MH)⁺.

Step 2. Synthesis of 1-(4-{[2-(3-ethyl-2-hydroxy-phenyl)-1-sulfo-ethylcarbamoyl]-methyl}-benzyl)-pyridinium formate. Prepared from the BCl₃ reaction of 1-(4-{[2-(3-tert-butoxycarbonyl-2-methoxy-phenyl)-1-(2,9,9-trimethyl-3,5-dioxa-4-bora-tricyclo[6.1.1.0^{2,6}]dec-4-yl)-ethylcarbamoyl]-methyl}-benzyl)-pyridinium chloride in DCM following the procedure described in Step 3 of Example 18. The crude product was purified by preparative HPLC using H₂O and MeOH solvents buffered with 0.1% formic acid to afford 32% of the product over two steps as a white solid. ESI-MS m/z 418 (MH−H₂O)⁺.

### Example 43

### (1R)-[3-{(2-hydroxy-ethylamino)-methyl}-benzoylamino]-(3-carboxy-2-hydroxy)benzyl-methylboronic acid formate

Step 1. Synthesis of 3-[2-{3-[(2-hydroxy-ethylamino)-methyl]-benzoylamino}-2-(2,9,9-trimethyl-3,5-dioxa-4-bora-tricyclo[6.1.1.0^{2,6}]dec-4-yl)-ethyl]-2-methoxy-benzoic acid tert-butyl ester. Prepared from the reductive amination of 3-[2-(3-formyl-benzoylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester with ethanolamine following the procedure described in Step 2 of Example 18. The crude product was taken to next step without further purification. ESI-MS m/z 607 (MH)⁺.

Step 2. Synthesis of (1R)-(3-(2-hydroxy-ethylamino)-methyl]-benzoyl amino-(3-carboxy-2-hydroxy)benzyl-methylboronic acid formate. Prepared from the BCl₃ reaction of 3-[2-{3-[(2-hydroxy-ethylamino)-methyl]-benzoylamino}-2-(2,9,9-trimethyl-3,5-dioxa-4-bora-tricyclo[6.1.1.0^{2,6}]dec-4-yl)-ethyl]-2-methoxy-benzoic acid tert-butyl ester in DCM following the procedure described in Step 3 of Example 18. The crude product was purified by preparative HPLC using H₂O and MeOH solvents buffered with 0.1% formic acid to afford 102 mg (32%) of the product as a white solid. ESI-MS m/z 385 (MH−H₂O)⁺.

### Example 44

### (2R)-3-{[2-(5-amino-pyridine-3-carbonyl)-amino]-2-borono-ethyl}-2-hydroxy-benzoic acid hydrochloride

Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and 5-tert-butoxycarbonylamino-nicotinic acid using the general procedure described in Steps 2-3 of Example 19 except that the final product was purified by C18 reverse phase chromatography eluting with 30% isopropanol (IPA)/water. ESI-MS m/z 328 (MH−H₂O)⁺.

### Example 45

### (2R)-3-{2-[(2-amino-pyridin-4-yl-acetyl)-amino]-2-borono-ethyl}-2-hydroxy-benzoic acid formate

Step 1. Synthesis of (2-chloro-pyridin-4-yl)-acetic acid ethyl ester. In a 500 mL round-bottom flask, diisopropylamine (13.2 mL, 93.92 mmol) was combined with THF (41 mL) and cooled to −78° C. Butyllithium (38 mL, 91.20 mmol) (2.5M in hexane) was added and the mixture was stirred for 30 minutes. 2-chloro-4-methylpyridine (4.1 mL, 46.92 mmol) was added, 17 mL THF was added, and the mixture was stirred for 2 hours. Diethylcarbonate (6.2 mL, 51.43 mmol) was added, and the mixture was stirred at −78° C. for overnight when temperature was slowly raised to ambient temperature. The reaction was quenched with saturated ammonium chloride and extracted thrice with ethyl acetate. The combined organic extracts were washed with brine, dried and evaporated. The crude oil was purified by silica gel chromatography, eluted using a gradient of 2/98(v/v) EtOAc/hexanes to 9/91 (v/v) EtOAc/hexanes to afford 8.7 g (93%) of product as clear oil. ESI-MS m/z 200 (MH)⁺.

Step 2. Synthesis of (2-tert-butoxycarbonylamino-pyridin-4-yl)-acetic acid ethyl ester. A 500 mL round-bottom-flask was charged with (2-chloro-pyridin-4-yl)-acetic acid ethyl ester (6.8 g, 34.0 mmol), tert-butyl carbamate (12.4 g, 105 mmol), 9,9-dimethyl-4,5-bis(diphenylphosphino)xanthene (4.2 g, 7.25 mmol), tris(dibenzylideneacetone)dipalladium (3.29 g, 3.59 mmol), cesium carbonate (16.9 g, 51.87 mmol) and THF (165 mL). The mixture was heated and refluxed under argon for 20 hours. Upon cooling, the reaction was quenched with 10% ammonium acetate solution and extracted with ethyl acetate. The combined organic extracts were washed with water, brine, dried and concentrated. The residue was purified by silicon gel chromatography, eluted using a gradient of 2/98(v/v) EtOAc/hexanes to 10/90 (v/v) EtOAc/hexanes to afford 14 g of product which was shown to be ca. 40% pure by HPLC. ESI-MS m/z 225 (MH−C₄H₈)⁺.

Step 3. Synthesis (2-tert-butoxycarbonylamino-pyridin-4-yl)-acetic acid. To a solution of (2-tert-butoxycarbonylamino-pyridin-4-yl)-acetic acid ethyl ester (14 g), methanol (45 mL), sodium hydroxide (4 g, 100 mmol), H₂O (45 mL) was stirred in reflux for 1.5 h. The solution was cooled and the methanol removed in vacuum. With stirring, 3N HCl was added to obtain a pH of between 4 and 5 resulting in the precipitation of white solids. The solids were filtered, and the filtrate concentrated in vacuo. The title product was purified by C18 reversed phase silica gel chromatography with eluted using a gradient of 100% H₂O to 95/5 (v/v) H₂O/IPA to afford 2.8 g (33%) of pale yellow solid. ESI-MS m/z 197 (MH−C₄H₈)⁺.

Step 4. Synthesis of 3-[2-[(2-tert-butoxycarbonylamino-pyridin-4-yl acetyl)-amino]-2-(2,9,9-trimethyl-3,5-dioxa-4-bora-tricyclo[6.1.1.0^{2,6}]dec-4-yl)-ethyl]-2-methoxy-benzoic acid tert-butyl ester. To anhydrous CH₂Cl₂ (0.41 mL, 6.5 mmol) in anhydrous THF (16 mL) under argon at −100° C. [MeOH, liq. N₂ slush bath], n-BuLi (2.4 mL, 2.5M in hexane, 6.0 mmol) was added dropwise and the mixture was stirred for 30 minutes. A THF (12 mL) solution of 2-methoxy-3-(2,9,9-trimethyl-3,5-dioxa-4-bora-tricyclo[6.1.1.0^{2,6}]dec-4-ylmethyl)-benzoic acid tert-butyl ester (2.0 g, 5.0 mmol) was added over a period of 20 minutes. After 30 minutes the cooling bath was removed and the mixture warmed slowly to 0° C. After 1 hour the reaction flask was cooled to −78° C., LHMDS (5.5 mL, 1M in THF, 5.5 mmol) was added slowly and the resultant solution was warmed to ambient temperature gradually while stirring overnight. Anhydrous MeOH (0.22 mL, 5.5 mmol) was added at −10° C., the reaction stirred for 1 h at −10° C. and then for 1 h at ambient temperature. At this stage LCMS indicated the formation of 2-methoxy-3-[2-(2,9,9-trimethyl-3,5-dioxa-4-bora-tricyclo[6.1.1.0^{2,6}]dec-4-yl)-2-(trimethylsilanyl-amino)-ethyl]-benzoic acid tert-butyl ester intermediate. Then all the solvent was pumped away in vacuo. The residue was redissolved in 50 mL of DCM.

In a separate dry round bottom flask with 2-(2-tert-butoxycarbonylamino)pyridin-4-yl)acetic acid (1.26 gm, 5.0 mmol), dry DCM (50 ml) was added. The contents in the flask were cooled to 0° C. NMM (1.65 mL, 15.0 mmol) was added followed by N-hydroxysuccinimide (NHS) (1.15 g, 10.0 mmol), and 1-(3-dimethylaminopropyl)-3-ethylcarbodiimide hydrochloride (EDCl) (1.92 g, 10.0 mmol). The mixture was stirred for 1 hour at 0° C. To this reaction mixture was added the solution of 2-methoxy-3-[2-(2,9,9-trimethyl-3,5-dioxa-4-bora-tricyclo[6.1.1.0^{2,6}]dec-4-yl)-2-(trimethylsilanyl-amino)-ethyl]-benzoic acid tert-butyl ester intermediate dropwise at 0° C. The cooling bath was removed and the reaction stirred at room temperature. After 2 hours the reaction was quenched with H₂O (100 mL) and the aqueous phase was extracted with DCM (3×100 mL), the combined organic layers were dried over sodium sulfite, and concentrated in vacuo. The crude product was purified by flash column chromatography silica gel eluted using a gradient of 35/65(v/v) EtOAc/hexanes to 50/50 (v/v) EtOAc/hexanes to give 300 mg of titled product. ESI-MS m/z 664 (MH)⁺.

Step 5. Synthesis of (2R)-3-{2-[(2-amino-pyridin-4-yl-acetyl)-amino]-2-borono-ethyl}-2-hydroxy-benzoic acid formate. To a solution of 3-[2-[(6-tert-butoxycarbonylamino-ylacetyl)-amino]-2-(2,9,9-trimethyl-3,5-dioxa-4-bora-tricyclo[6.1.1.0^{2,6}]dec-4-yl)-ethyl]-2-methoxy-benzoic acid tert-butyl ester (300 mg, 0.45 mmol) in DCM (3 ml) under argon was added BCl₃ (4.5 ml, 4.5 mmol, 1M solution in DCM) drop wise at −78° C. The mixture was stirred for 1 hr at −78° C. LCMS indicated the consumption of all of the starting material. At this point the reaction was quenched with water (10 ml) at 0° C. The DCM layer was evaporated. More water (10 ml) was added and the aqueous layer extracted with ether (3×15 ml). The aqueous layer was evaporated and the crude product was purified by preparative HPLC using solvents buffered with 0.1% formic acid to give 33 mg of resultant compound as a white solid in 20% yield. ESI-MS m/z 342 (MH−H₂O)⁺.

### Example 46

### (1R)-1-(6-amino-pyridin-3-yl-acetyl amino)-2-(2-hydroxy-3-carboxyphenyl)ethyl-1-boronic acid formate

Step 1. Synthesis of (6-chloropyridin-3-yl)acetonitrile. To a solution of 2-chloro-5-(chloromethyl)pyridine (25 g, 0.154 mol) in ethanol (40 mL) stirring at 0° C., was added a solution of sodium cyanide (8.17 g, 0.167 mol) in water (18 mL). The reaction mixture was refluxed for 2 hours then stirred at ambient temperature for a further 18 hours. The solvent was evaporated in vacuo and the residue extracted from water into DCM (500 mL), washed with brine, dried over sodium sulfate, filtered and evaporated in vacuo. The crude material was purified by column chromatography over silica gel eluting with 70/30 DCM/hexanes to afford 20 g (85%) of product as brown oil which solidified on standing. ESI-MS m/z 153 (MH)⁺.

Step 2. Synthesis of (6-chloropyridin-3-yl)acetic acid ethyl ester. 10 g (65.5 mmol) (6-chloropyridin-3-yl)acetonitrile were added to a mixture of 122 mL ethanol and 46 mL conc. sulfuric acid and the mixture stirred under reflux for 5 h. After cooling to ambient temperature, the reaction mixture was slowly added dropwise, while stirring, to a mixture of 161 g sodium bicarbonate and 450 mL water. The aqueous phase was extracted with DCM (three times with 300 mL each time). The combined organic phases were dried over sodium sulfate, filtered and concentrated on a rotary evaporator. The crude oil was purified by silica gel chromatography, eluted using a gradient of 2/98(v/v) EtOAc/hexanes to 9/91 (v/v) EtOAc/hexanes to afford 9.8 g (75%) of product as clear oil. ESI-MS m/z 200 (MH)⁺.

Step 3. Synthesis of (6-tert-butoxycarbonylamino-pyridin-3-yl)-acetic acid ethyl ester. A 500 mL round-bottom-flask was charged with 2-chloropyridin-3-yl)acetic acid ethyl ester (6.8 g, 34.0 mmol), tert-butyl carbamate (12.4 g, 105 mmol), 9,9-dimethyl-4,5-bis(diphenylphosphino)xanthene (4.2 g, 7.25 mmol), tris(dibenzylideneacetone)dipalladium (3.29 g, 3.59 mmol), cesium carbonate (16.9 g, 51.87 mmol) and THF (165 mL). The mixture was heated and refluxed under argon for 20 hours. Upon cooling, the reaction was quenched with 10% ammonium acetate solution and extracted with ethyl acetate. The combined organic extracts were washed with water, brine, dried and concentrated. The residue was purified by silica gel chromatography, eluted using a gradient of 2/98(v/v) EtOAc/hexanes to 10/90 (v/v) EtOAc/hexanes to afford 14 g of crude product. ESI-MS m/z 225 (MH−C₄H₈)⁺.

Step 4. Synthesis (6-tert-butoxycarbonylamino-pyridin-3-yl)-acetic acid. A solution of (6-tert-butoxycarbonylamino-pyridin-3-yl)-acetic acid ethyl ester (9.4 g), methanol (30 mL), sodium hydroxide (2.67 g, 100 mmol), and H₂O (30 mL) was stirred in reflux for 1.5 h. The solution was cooled and the methanol removed in vacuo. With stirring, 3N HCl was added to obtain a pH of between 4 and 5 resulting in the precipitation of pale yellow solids. The pale yellow solids were collected by filtration, washed by mixture of DCM and hexanes (v/v=1), brine and dried to afford 1.8 g (combined yield for Step 3 and 4 is 32%) of white solid. ESI-MS m/z 197 (MH−C₄H₈)⁺.

Step 5. Synthesis of 3-[2-[(6-tert-butoxycarbonylamino-pyridin-3-yl-acetyl)-amino]-2-(2,9,9-trimethyl-3,5-dioxa-4-bora-tricyclo[6.1.1.0^{2,6}]dec-4-yl)-ethyl]-2-methoxy-benzoic acid tert-butyl ester. Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and (6-tert-butoxycarbonylamino-pyridin-3-yl)-acetic acid using the general procedure described in Step 4 of Example 45. ESI-MS m/z 664 (MH)⁺.

Step 6. Synthesis (1R)-1-(6-amino-pyridin-3-yl-acetyl amino)-2-(2-hydroxy-3-carboxyphenyl)ethyl-1-boronic acid formate. Prepared from 3-[2-[(6-tert-butoxycarbonylamino-pyridine-3-acetyl)-amino]-2-(2,9,9-trimethyl-3,5-dioxa-4-bora-tricyclo[6.1.1.0^{2,6}]dec-4-yl)-ethyl]-2-methoxy-benzoic acid tert-butyl ester using the general procedure described in Step 5 of Example 45. ESI-MS m/z 342 (MH−H₂O)⁺.

### Example 47

### (2R)-3-[(2-[(2-chloro-pyridin-4-yl-acetyl)-amino]-2-borono-ethyl]-2-hydroxy-benzoic acid hydrochloride

Step 1. Synthesis (2-chloro-pyridin-4-yl)-acetic acid. To a solution of (2-chloro-pyridin-4-yl)-acetic acid ethyl ester prepared as described in Step 1 of Example 45, (4.98 g, 24.94 mmol), methanol (32 mL), sodium hydroxide (1.7 g, 42.50 mmol), H₂O (16 mL) was stirred in reflux for 2 h. The solution was cooled and all the solvent removed in vacuo. With stirring, 1N HCl was added to obtain a pH between 1 and 2 resulting in the precipitation of white solids. The solids were collected by filtration, washed with water and dried to afford 1.43 g (33%) of white solids. ESI-MS m/z 172 (MH)⁺.

Step 2. Synthesis of 3-[2-[(2-chloro-pyridin-4-yl acetyl)-amino]-2-(2,9,9-trimethyl-3,5-dioxa-4-bora-tricyclo[6.1.1.0^{2,6}]dec-4-yl)-ethyl]-2-methoxy-benzoic acid tert-butyl ester. Prepared from 2-methoxy-3-(2,9,9-trimethyl-3,5-dioxa-4-bora-tricyclo[6.1.1.0^{2,6}]dec-4-ylmethyl)-benzoic acid tert-butyl ester and (2-chloro-pyridin-4-yl)acetic acid following the general procedure described in Step 4 of Example 45. ESI-MS m/z 583 (MH)⁺.

Step 3. Synthesis of (2R)-3-{2-[(2-chloro-pyridin-4-yl-acetyl)-amino]-2-borono-ethyl}-2-hydroxy-benzoic acid hydrochloride. To a solution of 3-[2-[(2-chloro-pyridin-4-yl acetyl)-amino]-2-(2,9,9-trimethyl-3,5-dioxa-4-bora-tricyclo[6.1.1.0^{2,6}]dec-4-yl)-ethyl]-2-methoxy-benzoic acid tert-butyl ester (1.0 g, 1.71 mmol) in DCM (5 ml) under argon was added BCl₃ (15 ml, 15 mmol, 1M solution in DCM) drop wise at −78° C. The mixture was stirred for 1 hr at −78° C. LCMS indicated the consumption of all of the starting material. At this point the reaction was quenched with water (10 ml) at 0° C. The DCM layer was evaporated. More water (10 ml) was added and the aqueous layer extracted with ether (3×20 ml). The aqueous layer was evaporated and the crude product was purified by C18 reversed phase silica gel chromatography using a gradient of 1/99(v/v) IPA/H₂O to 3/97 (v/v) IPA/H₂O to give 53 mg of resultant compound as a white solid. ESI-MS m/z 361 (MH−H₂O)⁺.

### Example 48

### 3-{2(R)-[2-(2-amino-thiazol-4-yl)-2-(1-carboxy-1-methyl-ethoxyimino)-acetylamino]-2-borono-ethyl}-2-hydroxy-benzoic acid hydrobromide

Step 1. Synthesis of 2-{carboxy-[2-(trityl-amino)-thiazol-4-yl]-(Z)-methyleneaminooxy}-2-methyl-propionic acid tert-butyl ester. To a stirring mixture of 2-[(2-amino-thiazol-4-yl)-carboxy-(Z)-methyleneaminooxy]-2-methyl-propionic acid tert-butyl ester (3.3 g, 10 mmol) in methylene chloride (50 ml) was added diisopropylethylamine (4 ml, 23 mmol) and trityl chloride (6 g, 22 mmol). The mixture was stirred at room temperature overnight. After removal of solvent, the residue was dissolved in ethyl acetate (100 ml), washed with hydrochloric acid (0.01 N, 50 ml×3) and dried over magnesium sulfate. After removal of solvent, the residue was dissolved in ether and precipitated with hexane. This process was repeated one more time to yield 5 g (87%) of yellow solid. ESI-MS m/z 573 (MH)⁺.

Step 2. Synthesis of 3-[2(R)-{2-(1-tert-butoxycarbonyl-1-methyl-ethoxyimino)-2-[2-(trityl-amino)-thiazol-4-yl]-acetylamino}-2-(2,9,9-trimethyl-3,5-dioxa-4-bora-tricyclo[6.1.1.0^{2,6}]dec-4-yl)-ethyl]-2-methoxy-benzoic acid tert-butyl ester. To a stirring solution of anhydrous CH₂Cl₂ (0.48 mL, 7.5 mmol) in anhydrous THF (10 mL) under argon at −100° C. [methanol, dry ice/liq. N₂ slush bath] was added slowly n-BuLi (2.4 mL, 2.5M in hexane, 6 mmol) and the mixture was stirred at −100° C. for 30 minutes. A solution of 2-methoxy-3-(2,9,9-trimethyl-3,5-dioxa-4-bora-tricyclo[6.1.1.0^{2,6}]dec-4-ylmethyl)-benzoic acid tert-butyl ester (2 g, 5 mmol) in THF (4 mL) was added over a period of 10 minutes and the mixture was stirred at −100° C. for 5 minutes. The cooling bath was removed and the mixture was kept in an ice-bath for 1 hour. The reaction flask was then cooled to −78° C., lithium bis(trimethylsilyl)amide (LHMDS, 6 mL, 1M in THF, 6 mmol) was added slowly and the mixture was warmed to room temperature gradually while stirring overnight. Anhydrous methanol (0.22 mL, 5.4 mmol) was added at −10° C., the reaction mixture was stirred at room temperature for 1 hour. LCMS indicated the formation of 3-[2(R)-amino-2-(2,9,9-trimethyl-3,5-dioxa-4-bora-tricyclo[6.1.1.0^{2,6}]dec-4-yl)-ethyl]-2-methoxy-benzoic acid tert-butyl ester.

To a separate dry round bottom flask containing 2-{carboxy-[2-(trityl-amino)-thiazol-4-yl]-(Z)-methyleneaminooxy}-2-methyl-propionic acid tert-butyl ester (5 g, 5.2 mmol) and DMF (10 ml) was added NMM (2 ml, 18 mmol) and HATU (2 g, 5.2 mmol). The mixture was stirred at room temperature for 1 hour. Then the mixture was cooled in an ice-bath, the previously prepared solution of 3-[2(R)-amino-2-(2,9,9-trimethyl-3,5-dioxa-4-bora-tricyclo[6.1.1.0^{2,6}]dec-4-yl)-ethyl]-2-methoxy-benzoic acid tert-butyl ester was added. After stirring at room temperature for 2 h, the reaction was quenched with H₂O (10 mL). The organic solvent was removed and the aqueous phase was extracted with EtOAc (100 mL). The organic layer was washed with hydrochloric acid (0.01 N) and dried over MgSO₄. The crude product was purified by flash column chromatography (EtOAc/Hexane, 10-30%, v/v) to yield 0.353 g of yellow foam. ESI-MS m/z 984 (MH)⁺.

Step 3. Synthesis of 3-{2(R)-(2-(2-amino-thiazol-4-yl)-2-(1-carboxy-1-methyl-ethoxyimino)-acetylamino]-2-borono-ethyl}-2-hydroxy-benzoic acid hydrobromide. To a solution of 3-[2(R)-{2-(1-tert-butoxycarbonyl-1-methyl-ethoxyimino)-2-[2-(trityl-amino)-thiazol-4-yl]-acetylamino}-2-(2,9,9-trimethyl-3,5-dioxa-4-bora-tricyclo[6.1.1.0^{2,6}]dec-4-yl)-ethyl]-2-methoxy-benzoic acid tert-butyl ester (0.245 g, 0.25 mmol) in DCM (1 mL) at −78° C. was added BBr₃ (1.0 M in DCM, 2 mL, 2 mmol). The solution was stirred at room temperature for 6 h. The reaction was quenched with the slow addition of water (50 mL). After removal of organic solvent, the aqueous layer was extracted with ether (50 ml×3). The aqueous solution was loaded directly on a C18 reverse phase column and eluted with a gradient of 100% H₂O to 20% isopropanol (IPA)/H₂O. The combined fractions were concentrated and lyophilized to afford 15 mg of white powder. ESI-MS m/z 463 (MH−H₂O)⁺.

### Example 49

### 3-{2(R)-[2-(2-amino-thiazol-4-yl)-2(Z)-hydroxyimino-acetylamino]-2-borono-ethyl}-2-hydroxy-benzoic acid hydrochloride

Step 1. (2-tert-butoxycarbonylamino-thiazol-4-yl)-(Z)-trityloxyimino-acetic acid. This was prepared according to literature (Masaharu Kume, et al., The Journal of Antibiotics, 1993, 46, 177-192).

Step 2. 3-[2(R)-[2-(2-tert-butoxycarbonylamino-thiazol-4-yl)-2-(Z)-trityloxyimino-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester was prepared according to the procedure described in Step 2 of Example 48 by coupling (2-tert-butoxycarbonylamino-thiazol-4-yl)-(Z)-trityloxyimino-acetic acid with 3-[2(R)-amino-2-(2,9,9-trimethyl-3,5-dioxa-4-bora-tricyclo[6.1.1.0^{2,6}]dec-4-yl)-ethyl]-2-methoxy-benzoic acid tert-butyl ester. ESI-MS m/z 942 (MH)⁺.

Step 3. Synthesis of 3-{2(R)-(2-(2-amino-thiazol-4-yl)-2(Z)-hydroxyimino-acetylamino]-2-borono-ethyl}-2-hydroxy-benzoic acid hydrochloride. To a solution of 3-[2(R)-[2-(2-tert-butoxycarbonylamino-thiazol-4-yl)-2-(Z)-trityloxyimino-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester (0.934 g, 1 mmol) in DCM (5 mL) at −78° C. was added BCl₃ (1.0 M in DCM, 8 mL, 8 mmol). The solution was stirred at −78° C. for 2 h. The reaction was quenched with the slow addition of water (50 mL). After removal of organic solvent, the aqueous layer was extracted with ether (50 ml×3). The aqueous solution was loaded directly on a C18 reverse phase column and eluted with a gradient of 100% H₂O to 40% isopropanol (IPA)/H₂O. The combined fractions were concentrated and lyophilized to afford 57 mg of white powder. ESI-MS m/z 377 (MH−H₂O)⁺.

### Example 50

### 3-{2(R)-[2-[2-(2-amino-acetylamino)-thiazol-4-yl]-2(Z)-(1-carboxy-1-methyl-ethoxyimino)-acetylamino]-2-borono-ethyl}-2-hydroxy-benzoic acid hydrochloride

Step 1. 3-[2(R)-[2-(2-amino-thiazol-4-yl)-2(Z)-(1-tert-butoxycarbonyl-1-methyl-ethoxyimino)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester. This was prepared according to the procedure described in the Step 2 of Example 48 by coupling 2-[(2-amino-thiazol-4-yl)-carboxy-methyleneaminooxy]-2-methyl-propionic acid tert-butyl ester with 3-[2(R)-amino-2-(2,9,9-trimethyl-3,5-dioxa-4-bora-tricyclo[6.1.1.0^{2,6}]dec-4-yl)-ethyl]-2-methoxy-benzoic acid tert-butyl ester. ESI-MS m/z 742 (MH)⁺.

Step 2. Synthesis of 3-[2(R)-[2-[2-(2-tert-butoxycarbonylamino-acetylamino)-thiazol-4-yl]-2(Z)-(1-tert-butoxycarbonyl-1-methyl-ethoxyimino)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester. To an ice-cooled mixture of 3-[2(R)-[2-(2-amino-thiazol-4-yl)-2(Z)-(1-tert-butoxycarbonyl-1-methyl-ethoxyimino)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester (0.519 g, 0.7 mmol) and Boc-Gly-OH (0.127 mg, 0.72 mmol) in methylene chloride (10 ml) was added DCC (0.157 g, 0.76 mmol) and the mixture was stirred at room temperature for 2 hrs. The solid was filtered off and the filtrate was condensed. The crude product was purified by flash chromatography (EtOAc/hexane, 30-40%, v/v) to afford a white solid. ESI-MS m/z 900 (MH)⁺.

Step 3. 3-{2(R)-[2-[2-(2-amino-acetylamino)-thiazol-4-yl]-2(Z)-(1-carboxy-1-methyl-ethoxyimino)-acetylamino]-2-borono-ethyl}-2-hydroxy-benzoic acid hydrochloride. This was prepared according to the procedure described in Step 3 of Example 49 by de-protecting 3-[2(R)-[2-[2-(2-tert-butoxycarbonylamino-acetylamino)-thiazol-4-yl]-2(Z)-(1-tert-butoxycarbonyl-1-methyl-ethoxyimino)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester with BCl₃. ESI-MS m/z 521 (MH−H₂O)⁺.

### Example 51

### 3-(2(R)-{2-[2-(2-amino-acetylamino)-thiazol-4-yl]-2(Z)-methoxyimino-acetylamino}-2-borono-ethyl)-2-hydroxy-benzoic acid hydrochloride

Step 1. 3-[2(R)-[2-(2-amino-thiazol-4-yl)-2(Z)-methoxyimino-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester. This was prepared according to the procedure described in Step 2 of Example 48 by coupling (2-amino-thiazol-4-yl)-(Z)-methoxyimino-acetic acid with 3-[2(R)-amino-2-(2,9,9-trimethyl-3,5-dioxa-4-bora-tricyclo[6.1.1.0^{2,6}]dec-4-yl)-ethyl]-2-methoxy-benzoic acid tert-butyl ester. ESI-MS m/z 614 (MH)⁺.

Step 2. 3-[2(R)-{2-[2-(2-tert-butoxycarbonylamino-acetylamino)-thiazol-4-yl]-2(Z)-methoxyimino-acetylamino}-2-(2,9,9-trimethyl-3,5-dioxa-4-bora-tricyclo[6.1.1.0^{2,6}]dec-4-yl)-ethyl]-2-methoxy-benzoic acid tert-butyl ester. This was prepared according to the procedure described in Step 2 of Example 50 by coupling 3-[2(R)-[2-(2-amino-thiazol-4-yl)-2(Z)-methoxyimino-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester with Boc-Gly-OH.

Step 3. 3-(2(R)-{2-[2-(2-amino-acetylamino)-thiazol-4-yl]-2(Z)-methoxyimino-acetylamino}-2-borono-ethyl)-2-hydroxy-benzoic acid hydrochloride was prepared according to the procedure described in Step 3 of Example 49 by de-protecting 3-[2(R)-{2-[2-(2-tert-butoxycarbonylamino-acetylamino)-thiazol-4-yl]-2(Z)-methoxyimino-acetylamino}-2-(2,9,9-trimethyl-3,5-dioxa-4-bora-tricyclo[6.1.1.0^{2,6}]dec-4-yl)-ethyl]-2-methoxy-benzoic acid tert-butyl ester with BCl₃. ESI-MS m/z 449 (MH−H₂O)⁺.

### Example 52

### 3-(2(R)-{2-[2-(2(S),6-diamino-hexanoylamino)-thiazol-4-yl]-2(Z)-methoxyimino-acetylamino}-2-borono-ethyl)-2-hydroxy-benzoic acid hydrochloride

Step 1. 3-[2(R)-{2-[2-(2(S),6-bis-tert-butoxycarbonylamino-hexanoylamino)-thiazol-4-yl]-2(Z)-methoxyimino-acetylamino}-2-(2,9,9-trimethyl-3,5-dioxa-4-bora-tricyclo[6.1.1.0^{2,6}]dec-4-yl)-ethyl]-2-methoxy-benzoic acid tert-butyl ester. This was prepared according to the procedure described in Step 2 of Example 48 by coupling 3-[2(R)-[2-(2-amino-thiazol-4-yl)-2(Z)-methoxyimino-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester with Boc-Lys(Boc)-OH. ESI-MS m/z 942 (MH)⁺.

Step 2. 3-(2(R)-{2-[2-(2(S),6-diamino-hexanoylamino)-thiazol-4-yl]-2(Z)-methoxyimino-acetylamino}-2-borono-ethyl)-2-hydroxy-benzoic acid hydrochloride. This was prepared according to the procedure described in Step 3 of Example 49 by de-protecting 3-[2(R)-{2-[2-(2(S),6-bis-tert-butoxycarbonylamino-hexanoylamino)-thiazol-4-yl]-2(Z)-methoxyimino-acetylamino}-2-(2,9,9-trimethyl-3,5-dioxa-4-bora-tricyclo[6.1.1.0^{2,6}]dec-4-yl)-ethyl]-2-methoxy-benzoic acid tert-butyl ester with BCl₃. ESI-MS m/z 520 (MH−H₂O)⁺.

### Example 53

### 3-{2(R)-[2-(4-aminomethyl-phenyl)-2-hydroxyimino-acetylamino]-2-borono-ethyl}-2-hydroxy-benzoic acid hydrochloride

Step 1. Synthesis of benzyloxyimino-[4-(tert-butoxycarbonylamino-methyl)-phenyl]-acetic acid. A solution of [4-(tert-butoxycarbonylamino-methyl)-phenyl]-oxo-acetic acid (prepared according to U.S. Pat. No. 4,464,366, 1.136 g, 4 mmol) and O-benzyl-hydroxylamine (0.5 ml, 4.3 mmol) in ethanol (15 ml) was heated at 80° C. for 1 hr. After removal of solvent, 1.469 g (94%) of white foam was obtained. MS: 386 (MH)⁺.

Step 2. 3-[2(R)-{2-benzyloxyimino-2-[4-(tert-butoxycarbonylamino-methyl)-phenyl]-acetylamino}-2-(2,9,9-trimethyl-3,5-dioxa-4-bora-tricyclo[6.1.1.0^{2,6}]dec-4-yl)-ethyl]-2-methoxy-benzoic acid tert-butyl ester. This was prepared according to the procedure described in Step 2 of Example 48 by coupling benzyloxyimino-[4-(tert-butoxycarbonylamino-methyl)-phenyl]-acetic acid with 3-[2(R)-amino-2-(2,9,9-trimethyl-3,5-dioxa-4-bora-tricyclo[6.1.1.0^{2,6}]dec-4-yl)-ethyl]-2-methoxy-benzoic acid tert-butyl ester. ESI-MS m/z 797 (MH)⁺.

Step 3. 3-{2(R)-(2-(4-aminomethyl-phenyl)-2-hydroxyimino-acetylamino]-2-borono-ethyl}-2-hydroxy-benzoic acid hydrochloride was prepared according to the procedure described in Step 3 of Example 49 by de-protecting 3-[2(R)-{2-benzyloxyimino-2-[4-(tert-butoxycarbonylamino-methyl)-phenyl]-acetylamino}-2-(2,9,9-trimethyl-3,5-dioxa-4-bora-tricyclo[6.1.1.0^{2,6}]dec-4-yl)-ethyl]-2-methoxy-benzoic acid tert-butyl ester with BCl₃. ESI-MS m/z 384 (MH−H₂O)⁺.

### Example 54

### 3-[2(R)-(2-hydroxyimino-propionylamino)-2-borono-ethyl]-2-hydroxy-benzoic acid

Step 1. Synthesis of 2-benzyloxyimino-propionic acid. A suspension of sodium pyruvate (1.1 g, 10 mmol) and O-benzyl-hydroxylamine (1.16 ml, 10 mmol) in ethanol (30 ml) was heated at 80° C. for 3 hrs. After removal of solvent, the residue was dissolved in water (20 ml), adjusted to pH 2 with 1 N hydrochloric acid and extracted with ethyl acetate (25 ml×4). The combined extract was dried over magnesium sulfate. After removal of solvent, 1.85 g (96%) of white solid was obtained.

Step 2. 3-[2(R)-(2-benzyloxyimino-propionylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester. This was prepared according to the procedure described in Step 2 of Example 48 by coupling 2-benzyloxyimino-propionic acid with 3-[2(R)-amino-2-(2,9,9-trimethyl-3,5-dioxa-4-bora-tricyclo[6.1.1.0^{2,6}]dec-4-yl)-ethyl]-2-methoxy-benzoic acid tert-butyl ester. ESI-MS m/z 606 (MH)⁺.

Step 3. 3-[2(R)-(2-hydroxyimino-propionylamino)-2-borono-ethyl]-2-hydroxy-benzoic acid was prepared according to the procedure described in Step 3 of Example 49 by de-protecting 3-[2(R)-(2-benzyloxyimino-propionylamino)-2-(2,9,9-trimethyl-3,5-dioxa-4-bora-tricyclo[6.1.1.02^{2,6}]dec-4-yl)-ethyl]-2-methoxy-benzoic acid tert-butyl ester with BCl₃. ESI-MS m/z 293 (MH−H₂O)⁺.

### Example 55

### 3-[2(R)-(2-hydroxyimino-butyrylamino)-2-borono-ethyl]-2-hydroxy-benzoic acid

Step 1. 2-benzyloxyimino-butyric acid. This was prepared according to the procedure described in Step 1 of Example 54 by reacting 2-oxo-butyric acid with O-benzyl-hydroxylamine.

Step 2. 3-[2(R)-(2-benzyloxyimino-butyrylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester. This was prepared according to the procedure described in Step 2 of Example 48 by coupling 2-benzyloxyimino-butyric acid with 3-[2(R)-amino-2-(2,9,9-trimethyl-3,5-dioxa-4-bora-tricyclo[6.1.1.0^{2,6}]dec-4-yl)-ethyl]-2-methoxy-benzoic acid tert-butyl ester. ESI-MS m/z 620 (MH)⁺.

Step 3. 3-[2(R)-(2-hydroxyimino-butyrylamino)-2-borono-ethyl]-2-hydroxy-benzoic acid. This was prepared according to the procedure described in Step 3 of Example 49 by de-protecting 3-[2(R)-(2-benzyloxyimino-butyrylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester with BCl₃. ESI-MS m/z 307 (MH-H₂O)⁺.

### Example 56

### 3-[2(R)-(2-oxo-butyrylamino)-2-borono-ethyl]-2-hydroxy-benzoic acid

Step 1. Synthesis of 2-methoxy-3-[2(R)-(2-oxo-butyrylamino)-2-(2,9,9-trimethyl-3,5-dioxa-4-bora-tricyclo[6.1.1.0^{2,6}]dec-4-yl)-ethyl]-benzoic acid tert-butyl ester. To a solution of 2-oxo-butyric acid (0.55 g, 5.4 mmol) in methylene chloride (10 ml) was added DMF (0.4 ml, 5.2 mmol), and then added slowly oxalyl dichloride (0.52 ml, 6 mmol). After stirring at room temperature for 15 min, the solution was cooled in an ice-bath, NMM (0.6 ml, 5.5 mmol) was added followed by a solution of 3-[2(R)-amino-2-(2,9,9-trimethyl-3,5-dioxa-4-bora-tricyclo[6.1.1.0^{2,6}]dec-4-yl)-ethyl]-2-methoxy-benzoic acid tert-butyl ester prepared from 2-methoxy-3-(2,9,9-trimethyl-3,5-dioxa-4-bora-tricyclo[6.1.1.0^{2,6}]dec-4-ylmethyl)-benzoic acid tert-butyl ester (2 g, 5 mmol) according to the procedure described in Example 48. After stirring at room temperature for 2 h, the reaction was quenched with H₂O (10 mL). The organic solvent was removed and the aqueous phase was extracted with EtOAc (100 mL). The organic layer was washed with hydrochloric acid (0.01 N) and dried over MgSO₄. The crude product was purified by flash column chromatography (EtOAc/hexane, 15-25%, v/v) to yield 0.8 g (31%) of yellowish oil. ESI-MS m/z 515 (MH)⁺.

Step 2. 3-[2(R)-(2-oxo-butyrylamino)-2-borono-ethyl]-2-hydroxy-benzoic acid. This was prepared according to the procedure described in Step 3 of Example 49 by de-protecting 2-methoxy-3-[2(R)-(2-oxo-butyrylamino)-2-(2,9,9-trimethyl-3,5-dioxa-4-bora-tricyclo[6.1.1.0^{2,6}]dec-4-yl)-ethyl]-benzoic acid tert-butyl ester with BCl₃. ESI-MS m/z 310 (MH)⁺.

### Example 57

### (1R)-1-{2-[4-(pyridin-2-ylaminomethyl)-phenyl]-acetylamino}-1-[(3-carboxy-2hydroxy)benzyl]-methylboronic acid formate

Step 1. Synthesis of 3-[2-[2-(4-Formyl-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester. To anhydrous CH₂Cl₂ (3.1 mL, 48.7 mmol) in anhydrous THF (115 mL) under argon at −100° C. [MeOH, liq. N₂ slush bath], n-BuLi (18 mL, 2.5M in hexane, 44.9 mmol) was added dropwise and the mixture was stirred for 30 minutes. A THF (20 mL) solution of 2-Methoxy-3-(2,9,9-trimethyl-3,5-dioxa-4-bora-tricyclo[6.1.1.0^{2,6}]dec-4-ylmethyl)-benzoic acid tert-butyl ester (15.0 g, 37.4 mmol) was added over a period of 20 minutes. After 40 minutes the cooling bath was removed and the mixture warmed slowly to 0° C. After 1 hour the reaction flask was cooled to −78° C., LHMDS (41.2 mL, 1M in THF, 41.2 mmol) was added slowly and the resultant solution was warmed to room temperature gradually while stirring overnight. Anhydrous MeOH (1.67 mL, 41.2 mmol) was added at −10° C., the reaction stirred for 1 h at the same temperature and then for 1 h at room temperature. At this stage LCMS indicated the formation of 2-Methoxy-3-[2-(2,9,9-trimethyl-3,5-dioxa-4-bora-tricyclo[6.1.1.0^{2,6}]dec-4-yl)-2-(trimethylsilanyl-amino)-ethyl]-benzoic acid tert-butyl ester intermediate.

In a separate dry round bottom flask under argon containing (4-Formyl-phenyl)-acetic acid (7.2 gm, 44.2 mmol), dry DCM (80 ml) was added. The content in the flask were cooled to 0° C. NMM (4.9 mL, 44.9 mmol) was added followed by HATU (16.8 g, 44.2 mmol) and DMF (50 ml). The mixture stirred for 30 min at 0° C. and then 1 hr at room temperature. To this reaction mixture was added all of the reaction mixture containing 2-Methoxy-3-[2-(2,9,9-trimethyl-3,5-dioxa-4-bora-tricyclo[6.1.1.0^{2,6}]dec-4-yl)-2-(trimethylsilanyl-amino)-ethyl]-benzoic acid tert-butyl ester intermediate dropwise at −10° C. The cooling bath was removed and the reaction stirred at room temperature. After 2 h the reaction was quenched with H₂O (250 mL) and the aqueous phase was extracted with EtOAc (3×200 mL), the combined organic layers were dried over MgSO₄, and concentrated in vacuo. The crude product was dissolved in 1:1 mixture of hexane and methylene chloride (100 ml), and solid was filtered off. The filtrate which contained the desired compound was concentrated under vacuum and purified by flash column chromatography eluting initially with 30% ethyl acetate in hexane followed by changing the gradient to 35%, 40% and 50%.

[R_{f}=0.16, silica gel (EtOAc/Hexane, 50:50, v/v)] to give a 19% yield of product. ESI-MS m/z 576 (MH)⁺.

Step 2. Synthesis of 2-Methoxy-3-[2-{2-[4-(pyridin-2-ylaminomethyl)-phenyl]acetylamino}-2-(2,9,9-trimethyl-3,5-dioxa-4-bora-tricyclo[6.1.1.0^{2,6}]dec-4-yl)-ethyl]-benzoic acid tert-butyl ester. To a solution of 3-[2-[2-(4-Formyl-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester (370 mg, 0.64 mmol) in dichloroethane (4 ml) was added 2-amino-pyridine (91 mg, 0.96 mmol) followed by AcOH (78 mg, 1.3 mmol) and Na(OAc)₃BH (212 mg, 1.0 mmol). After stirring the reaction mixture for 15 hours at room temperature LCMS indicated the 80% consumption of the starting material. Solvent was removed under vacuum, water (20 ml) was added and the residues were extracted with EtOAc (3×50 mL), the combined organic layers were washed with saturated NaHCO₃ (10 ml) followed by brine (15 ml) and then dried over MgSO₄, and concentrated in vacuo. The crude product was taken to next reaction without further purification. ESI-MS m/z 654 (MH)⁺.

Step 3. Synthesis of (1R)-1-{2-[4-(pyridin-2-ylaminomethyl)-phenyl]-acetylamino}-1-[(3-carboxy-2hydroxy)benzyl]-methylboronic acid formate. To a solution of 2-Methoxy-3-[2-{2-[4-(pyridin-2-ylaminomethyl)-phenyl]-acetylamino}-2-(2,9,9-trimethyl-3,5-dioxa-4-bora-tricyclo[6.1.1.0^{2,6}]dec-4-yl)-ethyl]-benzoic acid tert-butyl ester (0.63 mmol) from the previous reaction in DCM (3 ml) under argon was added BCl₃ (6.3 ml, 6.3 mmol, 1M solution in DCM) dropwise at −78′C. The mixture was stirred for 1 hr at the same temperature then warmed to 0° C. After 1 hr of stirring at 0° C., LCMS indicated the consumption of all of the starting material. At this point reaction was quenched with water (10 ml) at 0° C. DCM layer was evaporated. More water (60 ml) was added and the aqueous layer extracted with ether (3×40 ml). The aqueous layer was evaporated to 15 mL, the pH of the aqueous layer was adjusted to 1.0, then it was purified by preparative HPLC using H₂O and MeOH solvents buffered with 0.1% formic acid to afford 8 mg of resultant compound as a white solid in 3% yield. ESI-MS m/z 432 (MH−H₂O)⁺.

### Example 58

### (1R)-1-(2-{4-[(1-Carboxymethyl-amino)-methyl]-phenyl}-acetylamino)1-[(3-carboxy-2-hydroxy)benzyl]-methylboronic acid formate

Step 1. Synthesis of 3-[2-(2-{4-[(tert-Butoxycarbonylmethyl-amino)-methyl]-phenyl}-acetylamino)-2-(2,9,9-trimethyl-3,5-dioxa-4-bora-tricyclo[6.1.1.0^{2,6}] dec-4-yl)-ethyl]-2-methoxy-benzoic acid tert-butyl ester. To a solution of 3-[2-[2-(4-Formyl-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester (370 mg, 0.64 mmol) in MeOH (6 ml) was added glycine tert-butyl ester hydrochloride (162 mg, 0.96 mmol) and AcOK (94 mg, 0.96 mmol). NaCNBH₃ (81 mg, 62.8 mmol) was then added at room temperature. At this point the reaction mixture became cloudy and it remained cloudy during the course of the reaction. After stirring the reaction mixture for 3 hours at room temperature LCMS indicated the complete consumption of the starting material. The solvent was removed under vacuum, water (20 ml) was added and the residues were extracted with EtOAc (3×50 mL), the combined organic layers were washed with saturated NaHCO₃ (10 ml) followed by brine (15 ml) and then dried over MgSO₄, and concentrated in vacuo. The crude product was taken to next reaction without further purification. ESI-MS m/z 691 (MH)⁺.

Step 2. Synthesis of (1R)-1-(2-{4-[(1-Carboxymethyl-amino)-methyl]-phenyl}-acetylamino)1-[(3-carboxy-2-hydroxy)benzyl]-methylboronic acid formate. Prepared from the BCl₃ reaction of 3-[2-(2-{4-[(tert-Butoxycarbonylmethyl-amino)-methyl]-phenyl}-acetylamino)-2-(2,9,9-trimethyl-3,5-dioxa-4-bora-ricyclo [6.1.1.0^{2,6}]dec-4-yl)-ethyl]-2-methoxy-benzoic acid tert-butyl ester in DCM following the procedure described in Step 3 of Example 57. The crude product was purified by preparative HPLC using H₂O and MeOH solvents buffered with 0.1% formic acid to afford 9% of the product over two steps as a white solid. ESI-MS m/z 413 (MH−H₂O)⁺.

### Example 59

### (1R)-1-(2-{4-[(1-Carboxy-4-guanidino-butylamino)-methyl]-phenyl}-acetylamino)-1-[(3-carboxy-2-hydroxy)benzyl]-methylboronic acid formate

Step 1. Synthesis of 3-[2-[2-(4-{[1-tert-Butoxycarbonyl-4-(N′-(4-methoxy-2,3,6-trimethylbenzenesulfonyl)-guanidino)-butylamino]-methyl}-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester. Prepared from the reductive amination of 3-[2-[2-(4-Formyl-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester (370 mg, 0.64 mmol) with N^{ω}-(4-methoxy-2,3,6-trimethylbenzenesulfonyl)-L-arginine-t-butylester following the procedure described in Step 2 of Example 57. The crude product was taken to next step without further purification. ESI-MS m/z 1002 (MH)⁺.

Step 2. (1R)-1-(2-{4-[(1-Carboxy-4-guanidino-butylamino)-methyl]-phenyl}-acetylamino)-1-[(3-carboxy-2-hydroxy)benzyl]-methylboronic acid formate. Prepared from the BCl₃ reaction of 3-[2-[2-(4-{[1-tert-Butoxycarbonyl-4-(N′-(4-methoxy-2,3,6-trimethylbenzenesulfonyl)-guanidino)-butylamino]-methyl}-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester in DCM following the procedure described in Step 3 of Example 1. The crude product was purified by preparative HPLC using H₂O and MeOH solvents buffered with 0.1% formic acid to afford 84 mg (17%) of the product over two steps as a white solid. ESI-MS m/z 512 (MH−H₂O)⁺.

### Example 60

### (1R)-1-(2-{4-[(1-Carboxy-2-hydroxy-ethylamino)-methyl]-phenyl}-acetylamino)-1-[(3-carboxy-2-hydroxy)benzyl]-methylboronic acid formate

Step 1. Synthesis 3-[2-(2-{4-[(2-Hydroxy-1-methoxycarbonyl-ethylamino)-methyl]-phenyl}-acetylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester. Prepared from the reductive amination of 3-[2-[2-(4-Formyl-phenyl)-acetylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester with DL-serine methylester hydrochloride following the procedure described in Step 1 of Example 258. The crude product was taken to next step without further purification. ESI-MS m/z 679 (MH)⁺.

Step 2. Synthesis of (1R)-1-(2-{4-[(1-Carboxy-2-hydroxy-ethylamino)-methyl]-phenyl}-acetylamino)-1-[(3-carboxy-2-hydroxy)benzyl]-methylboronic acid formate. Prepared from the BBr₃ reaction of 3-[2-(2-{4-[(2-Hydroxy-1-methoxycarbonyl-ethylamino)-methyl]-phenyl}-acetylamino)-2-(2,9,9-trimethyl-3,5-dioxa-4-bora-tricyclo[6.1.1.0^{2,6}]dec-4-yl)-ethyl]-2-methoxy-benzoic acid tert-butyl ester in DCM following the procedure described in Step 3 of Example 57. The crude product was purified by preparative HPLC using H₂O and MeOH solvents buffered with 0.1% formic acid to afford 40 mg (10%) of the product over two steps as a white solid. ESI-MS m/z 443 (MH−H₂O)⁺.

### Example 61

### (1R)-1-{2-[4-(1-guanidinomethyl)-phenyl]-acetylamino}-1-[(3-carboxy-2-hydroxy)benzyl]-methylboronic acid formate salt

To a solution of (1R)-1-{2-[4-(1-aminomethyl)-phenyl]-acetylamino}-1-[(3-carboxy-2-hydroxy)benzyl]-methylboronic acid formate (0.2 gm, 0.49 mmol), prepared as described in Example 30, in DMF (2 mL), N,N-diisopropylethylamine (DIPEA, 0.25 mL, 1.47 mmol) was added followed by 1H-1,2,4-triazole-1-carboxamidine hydrochloride (0.072 gm, 0.49 mmol) and the reaction stirred at room temperature for 3 h. Water (3 mL) was added to the reaction mixture, the solution was filtered and the filtrate purified by preparative HPLC to obtain a white solid in 18% yield. ESI-MS m/z 397 (MH−H₂O)⁺.

### Example 62

### (1R)-1-{2-[4-(Carbamimidoyl)-phenyl]-acetylamino}-1-[(3-carboxy-2-hydroxy)benzyl]-methylboronic acid formate salt

Step 1. Synthesis of (4-carbamimidoyl-phenyl)-acetic acid ethyl ester. A solution of p-cyanophenyl acetic acid (5 gm, 31 mmol) in ethanol (100 mL) was saturated with HCl gas and left to stir overnight. The solvent was removed in vacuo and the residue dried on high vacuum. To the white solid was added NH₃/EtOH (2 M, 100 mL) and the flask was tightly capped and left to stir overnight. The solution was concentrated in vacuo, and the resultant solids were triturated with diethyl ether. The solids were used without further purification.

Step 2. Synthesis of [4-(Benzyloxycarbonylamino-imino-methyl)-phenyl]-acetic acid ethyl ester. To (4-carbamimidoyl-phenyl)-acetic acid ethyl ester (8 gm, 38.8 mmol) in DCM (100 mL) was added triethylamine (12 mL, 85.36 mmol) and the solution cooled to 0° C. Benzylchloroformate (11 mL, 77.6 mmol) in DCM (20 mL) added dropwise and left to stir overnight. Water was added and the mixture extracted with DCM (3×50 mL). The combined organic layers were washed with aq. HCl, water, and brine. The solution was dried (Na₂SO₄) and concentrated in vacuo. Purification by silica gel chromatography afforded 2.2 g (21% over 2 steps) of solid.

Step 3. Synthesis of [4-(Benzyloxycarbonylamino-imino-methyl)phenyl]-acetic acid. To a solution of [4-(Benzyloxycarbonylamino-imino-methyl)-phenyl]-acetic acid ethyl ester (2.2 gm, 6.5 mmol) in methanol (20 mL) at 0° C., sodium hydroxide (9.7 mL, 1M/H₂O) was added dropwise and stirred for 2 hrs. The MeOH was removed in vacuo, water was added and the pH adjusted to acidic with dil. HCl, and the mixture extracted with EtOAc (3×50 mL). The combined organic layers were washed with water and brine, dried over sodium sulphate and evaporated to dryness to furnish 1.07 gm crude white solid which was used without further purification.

Step 4. Synthesis of 3-[2-[4-(Benzyloxycarbonylamino-imino-methyl)-benzoylamino]-2-(2,9,9-trimethyl-3,5-dioxa-4-bora-tricyclo[6.1.1.0^{2,6}]dec-4-yl)-ethyl]-2-methoxy-benzoic acid tert-butyl ester. To anhydrous CH₂Cl₂ (0.72 mL, 11.25 mmol) in anhydrous THF (20 mL) under argon at −100° C. [MeOH, liq. N₂ slush bath], n-BuLi (3.6 mL, 2.5M in hexane, 9 mmol) was added dropwise and the mixture was stirred for 30 minutes. A THF (12 mL) solution of 2-Methoxy-3-(2,9,9-trimethyl-3,5-dioxa-4-bora-tricyclo[6.1.1.0^{2,6}]dec-4-ylmethyl)-benzoic acid tert-butyl ester (3 g, 7.5 mmol) was added over a period of 20 minutes. After 40 minutes the cooling bath was removed and the mixture warmed slowly to 0° C. After 1 hour the reaction flask was cooled to −78° C., LHMDS (9 mL, 1M in THF, 9 mmol) was added slowly and the resultant solution was warmed to room temperature gradually while stirring overnight. Anhydrous MeOH (0.36 mL, 9 mmol) was added at −10° C., the reaction stirred for 1 h at the same temperature and then for 1 h at room temperature. At this stage LCMS indicated the formation of 2-Methoxy-3-[2-(2,9,9-trimethyl-3,5-dioxa-4-bora-tricyclo[6.1.1.02,6]dec-4-yl)-2-(trimethylsilanyl-amino)-ethyl]-benzoic acid tert-butyl ester intermediate.

In a separate dry round bottom flask under argon containing [4-(Benzyloxycarbonylamino-imino-methyl)phenyl]-acetic acid (1.07 gm, 3.43 mmol), dry DMF (10 mL) was added. The flask was cooled in an ice bath, pyridine (0.41 mL, 5.14 mmol) was added followed by HATU (1.56 gm, 4.11 mmol). The solution was stirred for 30 min at ° C. and then 30 min at ambient temperature. The mixture was cooled to 0° C. and the solution containing 2-Methoxy-3-[2-(2,9,9-trimethyl-3,5-dioxa-4-bora-tricyclo[6.1.1.02,6]dec-4-yl)-2-(trimethylsilanyl-amino)-ethyl]-benzoic acid tert-butyl ester was added. The cooling bath was removed and the reaction stirred at room temperature. After 2 h the reaction was quenched with H₂O (100 mL) and the aqueous phase was extracted with EtOAc (3×100 mL), the combined organic layers were dried over Na₂SO₄, and concentrated in vacuo. The crude product was purified by flash column chromatography [R_{f}=0.5, silica gel (EtOAc/Hexane, 50:50, v/v)] to give a 22% yield of product. ESI-MS m/z 724 (MH)⁺.

Step 5. Synthesis of 1-{2-[4-(Carbamimidoyl)-phenyl]-acetylamino}-1-[(3-carboxy-2-hydroxy)benzyl]-methylboronic acid formate salt. To a solution of 3-[2-[4-(Benzyloxycarbonylamino-imino-methyl)-benzoylamino]-2-(2,9,9-trimethyl-3,5-dioxa-4-bora-tricyclo[6.1.1.0^{2,6}]dec-4-yl)-ethyl]-2-methoxy-benzoic acid tent-butyl ester (0.560 gm, 0.77 mmol) in DCM (10 ml) under argon was added BCl₃ (7.7 ml, 7.7 mmol, 1M solution in DCM) dropwise at −78° C. The mixture was stirred for 1 hr at the same temperature then warmed to 0° C. After 1 hr of stirring at 0° C., LCMS indicated the consumption of all of the starting material. At this point the reaction was quenched with water (10 ml) at 0° C. The DCM layer was evaporated. Additional water (75 ml) was added and the aqueous layer extracted with ether (3×50 ml). The aqueous layer was evaporated to 20 mL then purified by preparative HPLC to give 31 mg of product as a white solid in 10.4% yield. ESI-MS m/z 368 (MH−H₂O)⁺.

### Example 63

### (2R)-3-{2-[(2-Aminomethyl-pyridin-4-yl-acetyl)-amino]-2-borono-ethyl}-2-hydroxy-benzoic acid hydrochloride

Step 1. Synthesis of (2-bromo-pyridin-4-yl)-acetic acid ethyl ester. In a 500 mL round-bottom flask, diisopropylamine (13.2 mL, 93.92 mmol) was combined with THF (41 mL) and cooled to −78° C. n-Butyllithium (2.5 M in hexane, 38 mL, 91.20 mmol) was added and the mixture was stirred for 30 minutes. 2-Bromo-4-methylpyridine (5 mL, 46.92 mmol) in 17 mL THF was added, and the mixture was stirred for 2 hours. Diethyl carbonate (6.2 mL, 51.43 mmol) was added, and the mixture was stirred overnight while gradually warming to room temperature. The reaction was quenched with saturated ammonium chloride and extracted thrice with ethyl acetate. The combined organic extracts were washed with brine, dried and evaporated. The crude oil was purified by silica gel chromatography, eluted using a gradient of 2/98(v/v) EtOAc/hexanes to 7/93 (v/v) EtOAc/hexanes to afford 8.01 g (70%) of product as a colorless oil. ESI-MS m/z 246 (MH)⁺.

Step 2. Synthesis of (2-cyano-pyridin-4-yl)-acetic acid ethyl ester. A 300 mL round-bottom-flask was charged with (2-bromo-pyridin-4-yl)-acetic acid ethyl ester (4.89 g, 20.0 mmol), zinc cyanide (9.94 g, 84.6 mmol), tetrakis(triphenylphosphine) palladium (0) (4.69 g, 4.06 mmol), and DMF (100 mL). The mixture was heated at 90° C. under Argon for 1.5 hours. Upon cooling, the reaction was quenched with 10% ammonium acetate solution and extracted with ethyl acetate. The combined organic extracts were washed with water, brine, dried and concentrated. The residue was purified by silica gel chromatography, eluted using a gradient of 2/98(v/v) EtOAc/hexanes to 10/90 (v/v) EtOAc/hexanes to afford 3.36 g (88.2%) of product. ESI-MS m/z 191 (MH)⁺.

Step 3. Synthesis of (2-aminomethyl-pyridin-4-yl)-acetic acid ethyl ester dihydrochloride. A mixture of 2-cyano-pyridin-4-yl)-acetic acid ethyl ester (4 g, 21.03 mmol), 10% palladium on carbon (2 g), and hydrogen chloride solution (15.7 mL of 4M in dioxane) in ethanol (140 mL) was charged with 60 psi of hydrogen in a Parr Shaker and stirred for 4 h. The mixture was filtered through Celite and the filtrate concentrated to afford 5.13 g (91%) of crude product which was used without further purification. ESI-MS m/z 195 (MH).⁺

Step 4. Synthesis of (2-tert-Butoxycarbonylaminomethyl-pyridin-4-yl)-acetic acid ethyl ester. To a solution of 40 mL tert-butanol and 13 mL acetone, (2-aminomethyl-pyridin-4-yl)-acetic acid ethyl ester dihydrochloride (5.13 g, 19.2 mmol), di-tert-butyldicarbonate (12.98 g, 59.5 mmol), sodium bicarbonate (3.225 g, 38.4 mmol), and 4-(dimethylamino)pyridine (DMAP, 513 mg, 4.20 mmol) was added. The mixture was stirred overnight at ambient temperature. The reaction was quenched with saturated ammonium chloride and extracted thrice with ethyl acetate. The combined organic extracts were washed with brine, dried and evaporated. The crude was purified by silica gel chromatography, eluted using a gradient of 20/80(v/v) EtOAc/hexanes to 30/70 (v/v) EtOAc/hexanes to afford 6.02 g (100%) of product as white solid. ESI-MS m/z 295 (MH)⁺.

Step 5. Synthesis of (2-tert-Butoxycarbonylaminomethyl-pyridin-4-yl)-acetic acid. To a solution of (2-tert-Butoxycarbonylaminomethyl-pyridin-4-yl)-acetic acid ethyl ester (6 g, 20.3 mmol) in methanol (20 mL) and H₂O (10 mL), sodium hydroxide (1.05 g, 26.2 mmol), was added. The mixture was stirred for 1.5 h during which time the solution became clear. The solvent was removed in vacuo, and with stirring 3N HCl was added dropwise to obtain a pH of between 4 and 5. The solvent was removed in vacuo, and the product was purified by C18 reversed phase silica gel chromatography with eluted using a gradient of 100% H₂O to 95/5 (v/v) H₂O/IPA to afford 5.42 g (100%) of a white solid. ESI-MS m/z 267 (MH)⁺.

Step 6. Synthesis of 3-[2-[(2-tert-Butoxycarbonylaminomethyl-pyridin-4-yl acetyl)-amino]-2-(2,9,9-trimethyl-3,5-dioxa-4-bora-tricyclo[6.1.1.0^{2,6}]dec-4-yl)-ethyl]-2-methoxy-benzoic acid tert-butyl ester. To anhydrous CH₂Cl₂ (1.80 mL, 28.50 mmol) in anhydrous THF (65 mL) under argon at −100° C. [MeOH, liq. N₂ slush bath], n-BuLi (10.5 mL, 2.5M in hexane, 26.3 mmol) was added slowly and the mixture was stirred for 30 minutes. A THF (11 mL) solution of 2-methoxy-3-(2,9,9-trimethyl-3,5-dioxa-4-bora-tricyclo[6.1.1.0^{2,6}]dec-4-ylmethyl)-benzoic acid tert-butyl ester (8.77 g, 21.93 mmol) was added over a period of 20 minutes. After 30 minutes the cooling bath was removed and the mixture warmed slowly to 0° C. After 1 hour the reaction flask was cooled to −78° C., LHMDS (24.1 mL, 1M in THF, 24.1 mmol) was added slowly and the resultant solution was warmed to ambient temperature gradually while stirring overnight. Anhydrous MeOH (0.96 mL, 24.1 mmol) was added at −10° C., the reaction stirred for 45 min at −10° C. and then for 1 h 15 min at ambient temperature. At this stage LCMS indicated the formation of 2-Methoxy-3-[2-(2,9,9-trimethyl-3,5-dioxa-4-bora-tricyclo[6.1.1.02,6]dec-4-yl)-2-(trimethylsilanyl-amino)-ethyl]-benzoic acid tert-butyl ester intermediate. Then all the solvent was removed in vacuo. The residue was dissolved in 140 mL of DCM.

In a separate dry round bottom flask with (2-tert-Butoxycarbonylaminomethyl-pyridin-4-yl)-acetic acid (5.84 gm, 21.93 mmol), dry DCM (220 mL) was added. The contents in the flask were cooled to 0° C. NMM (7.3 mL, 66.4 mmol) was added followed by N-Hydroxysuccinimide (NHS) (5.09, 44.2 mmol), 1-(3-Dimethylaminopropyl)-3-ethylcarbodiimide hydrochloride (EDCI) (8.54 g, 44.2 mmol). The mixture was stirred for 30 min at 0° C. and then 1 h at ambient temperature. To this reaction mixture was added all of the reaction mixture from the first part dropwise at 0° C. The cooling bath was removed and the reaction stirred at room temperature. After 1.5 hours the reaction was quenched with H₂O and the aqueous phase was extracted with DCM, the combined organic layers were dried over sodium sulfite, and concentrated in vacuo. The crude product was purified by flash column chromatography silica gel eluted using a gradient of 50/50(v/v) EtOAc/hexanes to 70/30 (v/v) EtOAc/hexanes to give 2.84 g (20%) of titled product. ESI-MS m/z 678 (MH)⁺.

Step 7. Synthesis of (2R)-3-{2-[(2-Aminomethyl-pyridin-4-yl-acetyl)-amino]-2-borono-ethyl}-2-hydroxy-benzoic acid hydrochloride. To a solution of 3-[2-[(6-tert-Butoxycarbonylaminomethyl-pyridylacetyl)-amino]-2-(2,9,9-trimethyl-3,5-dioxa-4-bora-tricyclo[6.1.1.0^{2,6}]dec-4-yl)-ethyl]-2-methoxy-benzoic acid tent-butyl ester (2.52 g, 3.72 mmol) in DCM (10 ml) under argon was added BCl₃ (38 ml, 38 mmol, 1M solution in DCM) dropwise at −78° C. The mixture was stirred for 1 hr at −78° C. LCMS indicated the consumption of the starting material. At this point the reaction was quenched with H₂O (30 mL) at 0° C. The DCM layer was evaporated. More H₂O (20 ml) was added and the aqueous layer extracted with ether (3×20 ml). The aqueous layer was evaporated and the title product was purified by C18 reversed phase silica gel chromatography using 100% H₂O to afford 770 mg (58%) of a white solid. ESI-MS m/z 356 (MH−H₂O)⁺.

### Example 64

### (2R)-3-{2-[(2-Guanidinomethyl-pyridin-4-yl-acetyl)-amino]-2-borono-ethyl}-2-hydroxy-benzoic acid formate

To a solution of (2R)-3-{2-[(2-Aminomethyl-pyridin-4-yl-acetyl)-amino]-2-borono-ethyl}-2-hydroxy-benzoic acid hydrochloride (74 mg, 0.18 mmol) in DMF (0.9 ml), 99 uL of DIPEA (0.54 mmol) and 28 mg of 1H-1,2,4-triazole-1-carboxamidine hydrochloride (0.18 mmol) was added sequentially. The mixture was stirred for 3 h at ambient temperature. LCMS indicated the consumption of the starting material. Water (5 mL) was added to the reaction, and the crude product was purified by preparative HPLC using solvents buffered with 0.1% formic acid to give 5 mg of product as a white solid. ESI-MS m/z 398 (MH−H₂O)⁺.

### Example 65

(1R)-1-{2-[3-(1-guanidinomethyl)-phenyl]-acetylamino}-1-[(3-carboxy-2-hydroxy)benzyl]-methylboronic acid formate salt

Prepared from (1R)-[2-(3-aminomethyl-phenyl)-acetylamino]-(3-carboxy-2hydroxy)benzyl-methylboronic acid formate salt using the procedure described in Example 61. ESI-MS m/z 397 (MH−H₂O)+.

Exemplary compounds of the present invention are shown in Table 1 along with respective molecular weights (MW) and low-resolution electrospray ionization mass spectral analytical results (ESI Mass Spec). The compounds of Table 1 are drawn as the open chain boronic acids, but as noted above there is a possibility that they can exist as cyclic boronate esters or as a mixture of the cyclic form and the open chain form as depicted in FIG. 6 (Strynadka et al., supra).

TABLE 1
Examples of compounds of the present invention.

| Example | Structure | MW | ESI-MS m/z |
|---|---|---|---|
| 1 | 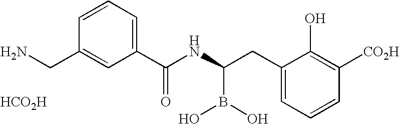 | 404.2 | 341 (MH − H₂O)+ |
| 2 | 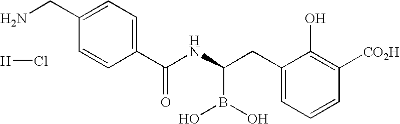 | 394.7 | 341 (MH − H₂O)+ |
| 3 | 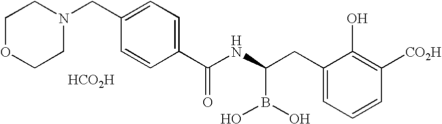 | 474.3 | 411 (MH − H₂O)+ |
| 4 | 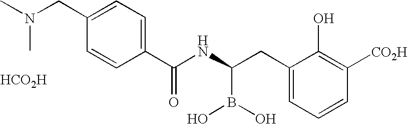 | 432.2 | 369 (MH − H₂O)+ |
| 5 | 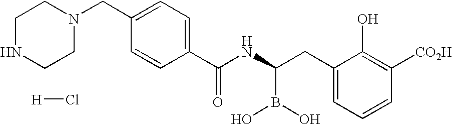 | 463.8 | 411 (MH − H₂O)+ |
| 6 | 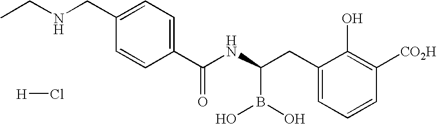 | 422.7 | 369 (MH − H₂O)+ |
| 7 | 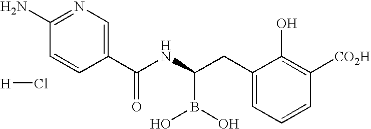 | 345.1 | 328 (MH − H2O)+ |
| 8 | 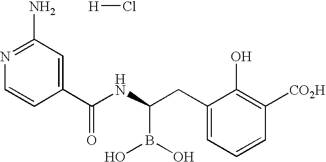 | 345.1 | 328 (MH − H2O)+ |
| 9 | 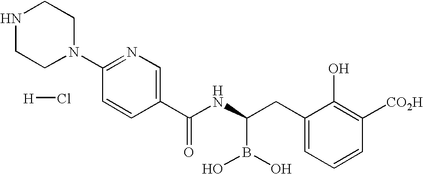 | 450.7 | 397 (MH − H2O)+ |
| 10 | 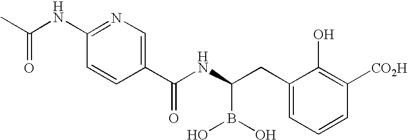 | 387.2 | 370 (MH − H2O)+ |
| 11 | 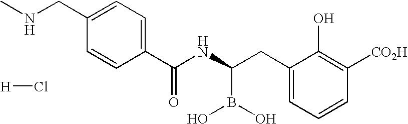 | 408.7 | 355 (MH − H2O)+ |
| 12 | 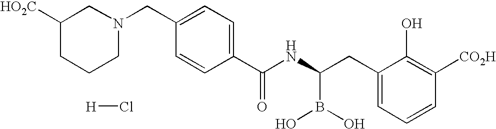 | 506.8 | 454 (MH − H2O)+ |
| 13 | 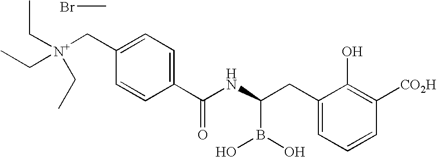 | 524.2 | 426 (MH − H2O)+ |
| 14 | 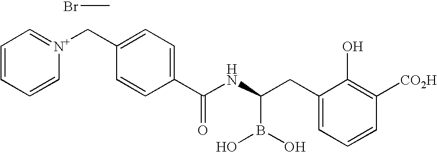 | 516.2 | 404 (MH − H2O)+ |
| 15 | 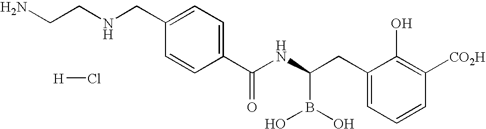 | 436.8 | 384 (MH − H2O)+ |
| 16 | 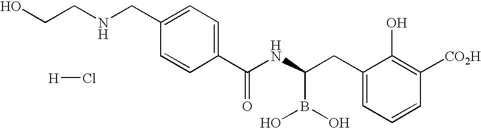 | 438 | 386 (MH − H2O)+ |
| 17 | 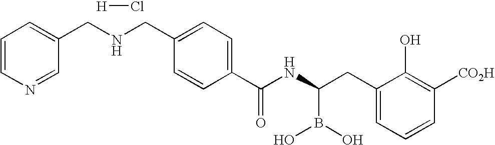 | 485.8 | 433 (MH − H2O)+ |
| 18 | 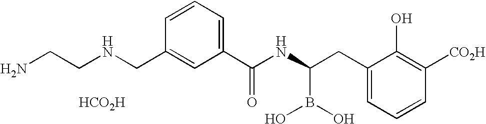 | 417 2 | 384 (MH − H2O)+ |
| 19 | 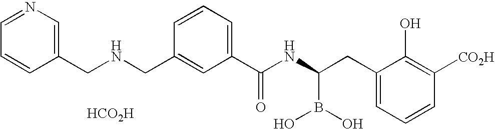 | 495.3 | 432 (MH − H2O)+ |
| 20 | 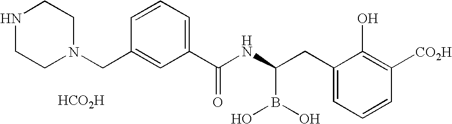 | 473.3 | 411 (MH − H2O)+ |
| 21 | 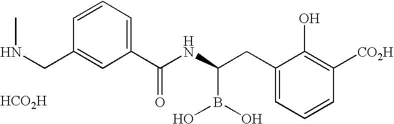 | 418.2 | 355 (MH − H2O)+ |
| 22 | 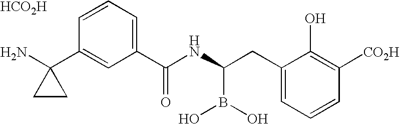 | 430.2 | 367 (MH − H2O)+ |
| 23 | 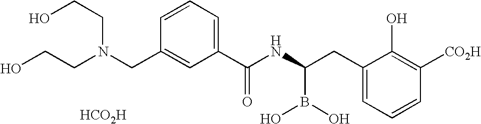 | 492.3 | 429 (MH − H2O)+ |
| 24 | 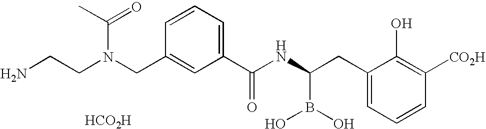 | 489.3 | 426 (MH − H2O)+ |
| 25 | 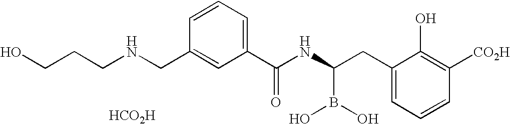 | 462.2 | 399 (MH − H2O)+ |
| 26 | 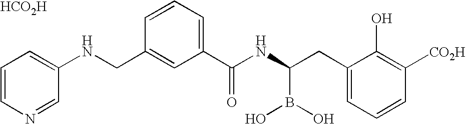 | 481.3 | 418 (MH − H2O)+ |
| 27 | 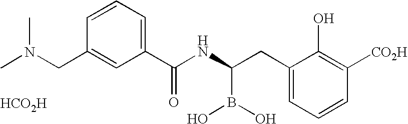 | 432.2 | 369 (MH − H2O)+ |
| 28 | 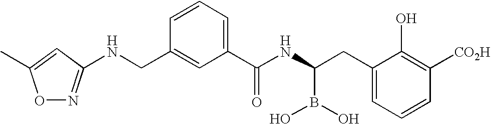 | 439 | 422 (MH − H2O)+ |
| 29 | 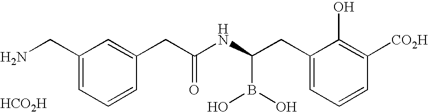 | 418.2 | 355 (MH − H2O)+ |
| 30 | 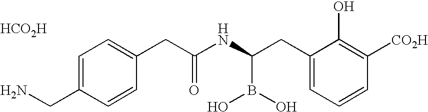 | 418.2 | 355 (MH − H2O)+ |
| 31 | 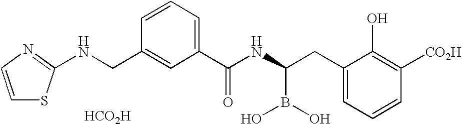 | 487.3 | 424 (MH − H2O)+ |
| 32 | 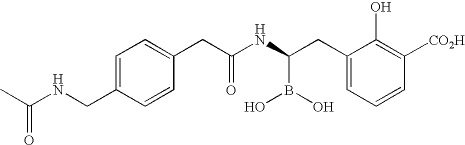 | 414 | 397 (MH − H2O)+ |
| 33 | 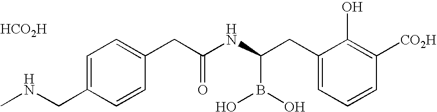 | 432.2 | 369 (MH − H2O)+ |
| 34 | 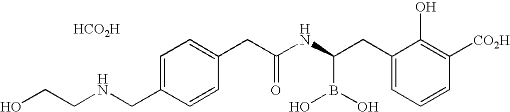 | 462.2 | 399 (MH − H2O)+ |
| 35 | 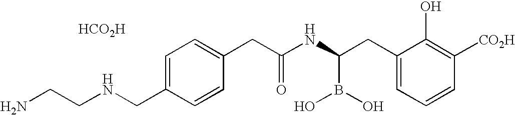 | 461.2 | 398 (MH − H2O)+ |
| 36 | 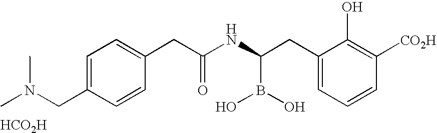 | 446.2 | 383 (MH − H2O)+ |
| 37 | 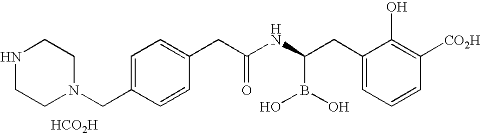 | 487.3 | 424 (MH − H2O)+ |
| 38 | 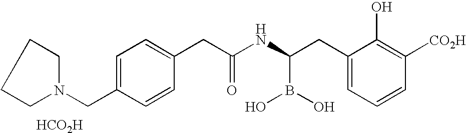 | 506.3 | 409 (MH − H2O)+ |
| 39 | 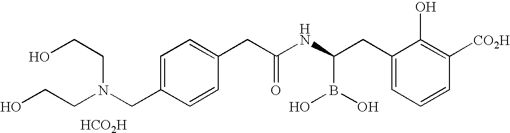 | 486.3 | 443 (MH − H2O)+ |
| 40 | 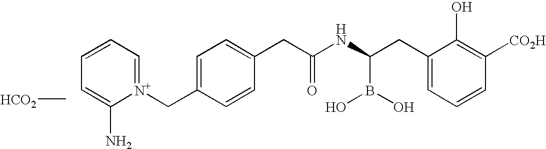 | 496.3 | 433 (MH − H2O)+ |
| 41 | 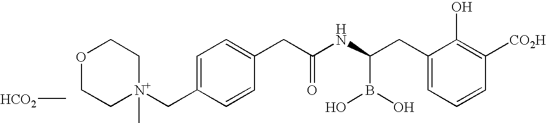 | 503.3 | 440 (MH − H2O)+ |
| 42 | 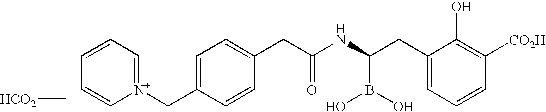 | 481.3 | 418 (MH − H2O)+ |
| 43 | 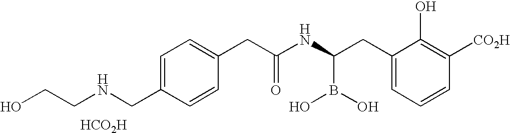 | 448 2 | 385 (MH − H2O)+ |
| 44 | 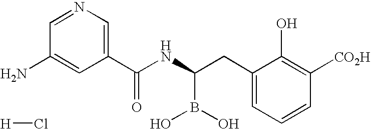 | 381.6 | 328 (MH − H2O)+ |
| 45 | 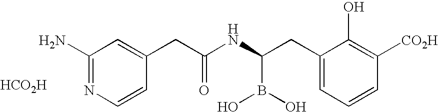 | 405.2 | 342 (MH − H2O)+ |
| 46 | 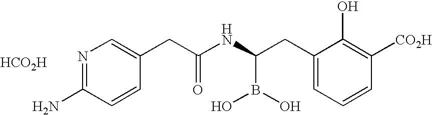 | 405.2 | 342 (MH − H2O)+ |
| 47 | 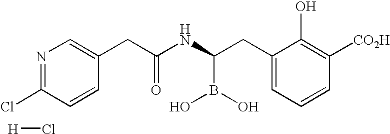 | 378.6 | 361 (MH − H2O)+ |
| 48 | 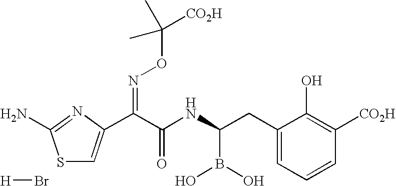 | 561.1 | 463 (MH − H2O)+ |
| 49 | 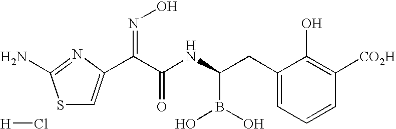 | 430.7 | 377 (MH − H2O)+ |
| 50 | 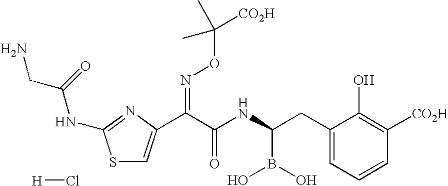 | 573.8 | 520 (MH − H2O)+ |
| 51 | 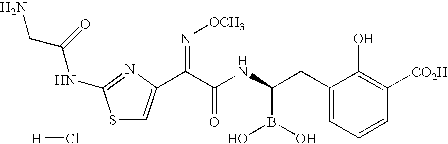 | 501.7 | 449 (MH − H2O)+ |
| 52 | 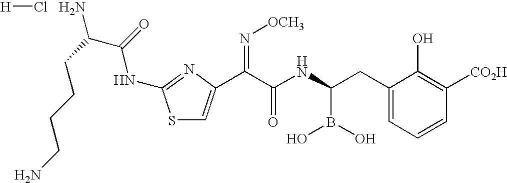 | 570 9 | 520 (MH − H2O)+ |
| 53 | 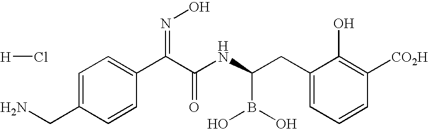 | 437.7 | 384 (MH − H2O)+ |
| 54 | 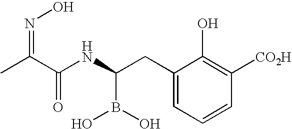 | 310.1 | 292 (MH − H2O)+ |
| 55 | 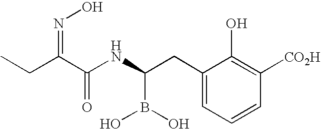 | 324.1 | 307 (MH − H2O)+ |
| 56 | 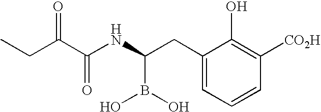 | 309.1 | 310 (MH+) |
| 57 | 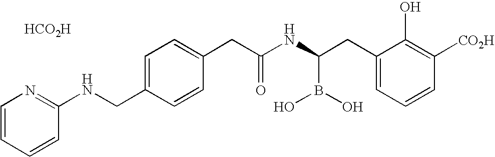 | 495.3 | 432 (MH − H2O)+ |
| 58 | 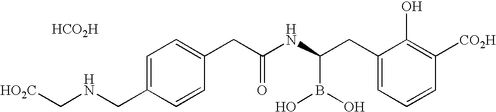 | 476.2 | 413 (MH − H2O)+ |
| 59 | 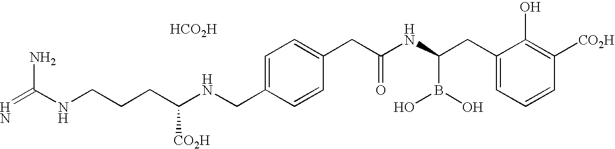 | 575.2 | 512 (MH − H2O)+ |
| 60 | 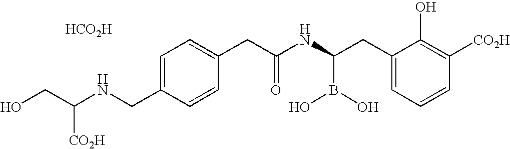 | 506.2 | 443 (MH − H2O)+ |
| 61 | 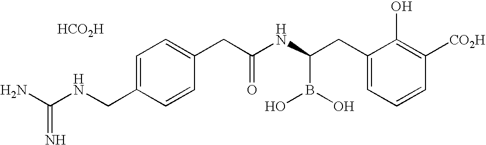 | 460.2 | 397 (MH − H2O)+ |
| 62 | 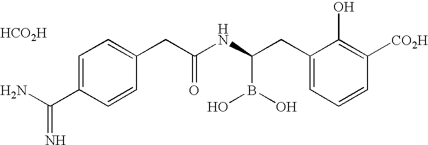 | 432.2 | 368 (MH − H2O)+ |
| 63 | 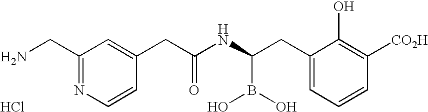 | 410.7 | 356 (MH − H2O)+ |
| 64 | 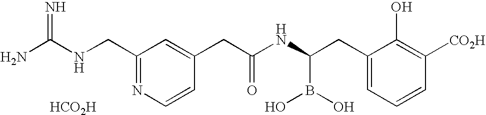 | 461.2 | 398 (MH − H2O)+ |
| 65 | 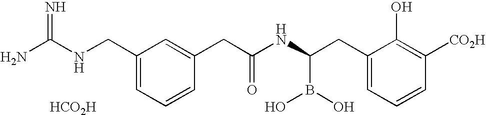 | 460.2 | 397 (MH − H2O)+ |

### Example 66

### Experimental Method for β-Lactamase Enzyme Assays

Isolation of β-lactamases. For SHV-5, p99 AmpC, KPC-2 and CTX-M15 β-lactamases, E. coli BL21(DE3) bacterial cells carrying expression plasmids (expressed as native untagged proteins) for the individual β-lactamases were grown in 1 L of Superbroth (Teknova Inc. Hollister, Calif.) supplemented with 100 μg/ml kanamycin and 1×5052 (0.5% glycerol, 0.05% glucose and 0.2% α-lactose) at 35° C. for 18-20 hours. Cells were harvested by centrifugation (4,000×g, 4° C., 20 min), and resuspended in 50 ml of 10 mM HEPES pH 7.5 (1/20 of the initial volume). The cells were lysed by sonication (5 pulses of 45 seconds) at 45 W on ice. The lysates were clarified by centrifugation at 10,000×g for 40 minutes at 4° C. Samples were diluted 5-fold in 50 mM sodium acetate pH 5.0, stored overnight at 4° C., after which they were centrifuged at 10,000×g for 30 minutes to clarify, and filtered through 0.45 μm filters. The samples were loaded onto a 5 ml Capto S sepharose cation exchange column (GE Healthcare) pre-equilibrated with 50 mM sodium acetate pH 5.0. The column was washed with 5 column volumes of 50 mM sodium acetate pH 5.0 to wash out unbound protein and a linear gradient of NaCl (0 to 500 mM) was used to elute the protein (over 16 CV) from the column. Fractions were assayed for β-lactamase activity using Centa (Calbiochem, Gibbstown, N.J.) as a reporter β-lactamase substrate for activity in the isolated fractions. Active fractions were pooled, concentrated and further purified by gel filtration chromatography on a Superdex 75 prep grade gel filtration column (GE Healthcare, Piscataway, N.J.) pre-equilibrated in 50 mM Hepes pH 7.5, 150 mM NaCl. Active fractions were pooled concentrated, quantitated by BCA protein determination (Thermo Scientific, Rockford, Ill.), and frozen at −80° C. in 20% glycerol until use.

For VIM-2 metallo (β-lactamase, the procedure was identical with the following exceptions: 1) the protein was not pH adjusted to pH 5 with 50 mM sodium acetate, 2) the chromatography step was changed to a 5 ml Q sepharose anion exchange column pre-equilibrated with 50 mM Hepes pH 7.5, and 3) elution of the protein was achieved by a linear gradient of NaCl (0-600 mM). Finally, the VIM-2 purification required a second run (3^{rd} step) on the Q sepharose anion exchange column to achieve acceptable purity (>90%).

For OXA-23 β-lactamase, crude β-lactamase extracts were prepared from 20 ml overnight cultures with shaking. Acenitobacter baumanni cells containing OXA-23 were further diluted 10-fold and grown to mid-log phase (OD at 600 nm, 0.5-0.8) in Mueller-Hinton II (MH-II) broth at 37° C. The cells were pelleted at 5000 g, washed and resuspended in 2 mL PBS pH 7.0. The β-lactamases were extracted by four cycles of freezing and thawing followed by centrifugation. β-lactamase activity in the extracts was measured with the chromogenic cephalosporin nitrocefin. The amount of protein in each β-lactamase preparation was determined by the bicinchoninic acid (BCA) assay.

β-lactamase Inhibition. To determine the level of inhibition of β-lactamase enzymes, compounds were diluted in PBS at pH 7.0 to yield concentrations between 100 and 0.005 μM in microtiter plates. An equal volume of diluted enzyme stock was added, and the plates were incubated at 37° C. for 10 min. Nitrocefin solution was then dispensed as substrate into each well at a final concentration of 100 μM, and the plates were immediately read with the kinetic program at 486 nm for 10 min on the SPECTRAMAX® Plus³⁸⁴ (high-throughput microplate spectrophotometer; Molecular Devices Corp., Sunnyvale, Calif.). Maximum rates of metabolism were then compared to those in control wells (without inhibitors), and percentages of enzyme inhibition were calculated for each concentration of inhibitor. The concentration of inhibitor needed to reduce the initial rate of hydrolysis of substrate by 50% (IC₅₀) was calculated as the residual activity of β-lactamase at 486 nm using the SoftMax Pro 5.0 software (Molecular Devices Corp.).

Using the methodology described above, examples of the current invention were evaluated for their ability to inhibit β-lactamase enzymes. The results of these assays are summarized in Table 2 for representative enzymes across different subtypes (note SHV-5 and CTXM-15 exemplify different subclasses of Ambler Class A Extended Spectrum Beta Lactamases, KPC-2 exemplifies Class A carbapenemases, P99 represents chromosomal Class C AmpC, VIM-2 exemplifies subclass B1 of metallo-β-lactamases, and OXA-23 represents chromosome-encoded Class D oxacillin-hydrolyzing β-lactamases), NT=not tested.

TABLE 2
Inhibition of diverse β-lactamases by example compounds of the present
invention.

| | SHV-5 | CTXM-15 | P99 AmpC | KPC-2 | VIM-2 IC₅₀ | OXA-23 |
|---|---|---|---|---|---|---|
| Example | IC₅₀ ((nM) | IC₅₀ (nM) | IC₅₀ (nM) | IC₅₀ (nM) | (nM) | IC₅₀ (nM) |
| 1 | 653 | 3 | 1 | 20 | 152 | 64 |
| 2 | >1000 | 2 | 3 | 13 | 22 | >1000 |
| 3 | 367 | 4 | 3 | 14 | 850 | >1000 |
| 4 | 0.6 | 3 | 3 | 4 | >1000 | 99 |
| 5 | >1000 | 6 | 1 | 1 | 510 | >1000 |
| 6 | >1000 | 4 | 1 | 4 | 57 | >1000 |
| 7 | 320 | 1 | 1 | 4 | 820 | 508 |
| 8 | 29 | 28 | 2 | 5 | 700 | 144 |
| 9 | >1000 | 83 | 2 | 6 | 242 | >1000 |
| 10 | 319 | 3 | 3 | 4 | 680 | 580 |
| 11 | >1000 | 4 | 1 | 4 | 57 | >1000 |
| 12 | 870 | 3 | 900 | 11 | 930 | >1000 |
| 13 | >1000 | 14 | 4 | 3 | 340 | >1000 |
| 14 | >1000 | 1 | 29 | 21 | 360 | >1000 |
| 15 | >1000 | 7 | 5 | 18 | 47 | >1000 |
| 16 | >1000 | 5 | 6 | 5 | NT | NT |
| 17 | >1000 | 5 | 8 | 15 | NT | NT |
| 18 | 257 | 1 | 0.3 | 14 | 99 | 41 |
| 19 | 54 | 1 | 0.2 | 6 | 510 | 19 |
| 20 | 42 | 20 | 0.3 | 9 | 179 | 22 |
| 21 | 830 | 3 | 2 | 19 | 142 | 33 |
| 22 | >1000 | 0.6 | 34 | 5 | NT | NT |
| 23 | 17 | 2 | 0.9 | 8.1 | NT | NT |
| 24 | 350 | 4 | 2 | 18 | NT | NT |
| 25 | 580 | 3 | 0.9 | 20 | NT | NT |
| 26 | 94 | 1 | 0.4 | 11 | NT | NT |
| 27 | 312 | 3 | 1.4 | 14 | NT | NT |
| 28 | 15 | 0.7 | 0.2 | 4 | 304 | 9 |
| 29 | 5 | 9 | 6 | 16 | 882 | 591 |
| 30 | 3 | 7 | 12 | 9 | 271 | 252 |
| 31 | 13 | 0.5 | 0.6 | 3 | 295 | 10 |
| 32 | 0.3 | 2 | 2 | 4 | >1000 | 116 |
| 33 | 5 | 8 | 7 | 12 | 336 | 379 |
| 34 | 5 | 7 | 4 | 18 | 580 | 256 |
| 35 | 2 | 7 | 5 | 17 | 196 | 97 |
| 36 | 2 | 4 | 2 | 5 | 593 | 191 |
| 37 | 2 | 7 | 2 | 9 | 785 | 83 |
| 38 | 5 | 3 | 9 | 6 | 858 | 234 |
| 39 | 970 | 4 | 12 | 26 | 469 | 167 |
| 40 | 20 | 4 | 10 | 653 | >1000 | 132 |
| 41 | 4 | 2 | 20 | 10 | >1000 | 201 |
| 42 | 2 | 4 | 4 | 10 | >1000 | 153 |
| 43 | 4 | 12 | 9 | 70 | 184 | 38 |
| 44 | 70 | 2 | 1 | 3D | 690 | 120 |
| 45 | 1D | 0.5 | 2 | 6 | 811 | 113 |
| 46 | 0.7 | 5 | 4 | 3 | NT | 209 |
| 47 | 1 | 2 | 5 | 2 | >1000 | 132 |
| 48 | 4 | 12 | 10 | 70 | >1000 | 670 |
| 49 | 4 | 11 | 2 | 34 | 590 | 17 |
| 50 | 24 | 34 | 23 | 158 | NT | NT |
| 51 | 15 | 40 | 17 | 58 | NT | NT |
| 52 | 26 | 55 | 8 | 89 | NT | NT |
| 53 | 29 | 12 | NT | 39 | 312 | 350 |
| 54 | 48 | 5 | 2 | 10 | >1000 | 494 |
| 55 | 120 | 1 | 0.5 | 6 | >1000 | 67 |
| 56 | 195 | 11 | 2 | 24 | >1000 | 31 |
| 57 | 2 | 4 | 3 | 13 | NT | 156 |
| 58 | 0.5 | 4 | 6 | 13 | NT | 182 |
| 59 | 2 | NT | 6 | 15 | NT | NT |
| 60 | 2 | NT | 9 | 11 | NT | NT |
| 61 | 2 | 4 | 11 | 14 | NT | NT |
| 62 | 2 | NT | 3 | 9 | NT | NT |
| 63 | 8 | 10 | 9 | 24 | 621 | 534 |
| 64 | 3 | 3 | 8 | 17 | 548 | 177 |
| 65 | NT | NT | NT | NT | NT | NT |

### Example 67

### In vitro Antibacterial Assays of β-Lactamase Inhibition

To determine the ability of test compounds to potentiate the inhibition of the growth of bacterial strains producing beta-lactamase enzymes, classic cell based screening assays were employed. Five bacteria strains producing beta-lactamase enzymes were used: K. pneumoniae expressing the Class A Extended Spectrum Beta-Lactamase (ESBL) CTX-M-15, E. coli expressing the Class A ESBL SHV-5, E. cloacae expressing the Class C P99+, K. pneumoniae expressing the Class A carbapenemase KPC-2, and P. aeruginosa expressing the Class B metallo β-lactamase VIM-2. In order to evaluate the ability of test compounds to inhibit beta-lactamase activity, Applicants used a modification of the broth microdilution assay. The assay was conducted in Cation Adjusted Mueller Hinton Broth (CAMHB, BD # 212322, BD Diagnostic Systems, Sparks, Md.). Bacteria strains were grown for 3-5 hours in CAMBH broth. All five strains were grown in presence of 50 μg/mL ampicillin to ensure resistance is maintained. In the meantime, test compounds were diluted in DMSO to a 0.1 mg/mL stock. The compounds were added to a microtiter plate and were diluted in 2-fold serial dilutions in CAMHB in a final concentration range of 8 μg/mL to 0.015 μg/ml. For Examples 1-56, an overlay of CAMHB containing the cephalosporin Ceftazidime was added to the compounds at a final static concentration of 8 μg/ml, except for the P. aeruginosa expressing VIM-2 which used an overlay of 16 μg/mL. Ceftazidime (CAZ) has the following MIC's for the bacteria strains used: for K. pneumoniae expressing Ambler Class A ESBL CTX-M-15 the MIC alone=128 μg/ml, E. coli expressing Class A ESBL SHV-5 the MIC alone >1024 μg/mL, K. pneumoniae expressing Ambler Class A carbapenemase KPC-2 the MIC alone=32 μg/mL, E. cloacae expressing Class C P99+ AmpC the MIC alone=128 μg/mL, and P. aeruginosa expressing Class B VIM-2 the MIC alone=128 μg/mL. For Examples 57-65 an overlay of CAMBH containing the penicillin derivative Piperacillin (Pip) was added to the compounds at a final static concentration of 16 μg/ml. The MIC alone for Pip against all of the strains tested was >128 μg/mL. Titration of test compounds with MIC readout indicates the concentration of test article needed to sufficiently inhibit beta lactamase enzyme activity and protect the intrinsic antibacterial activity of the cephalosporin. Each of these compound plates are made in quadruplicate, one for each bacteria strain. In addition to the titration of test compounds the MICs of a panel of cephalosporins is also tested to ensure the strains are behaving consistently from test to test. Once the test compound and cephalosporin are added the plates can be inoculated. Inocula are conducted according to CLSI broth microdilution method. After inoculation the plates are incubated for 16-20 hours at 37° C. then the Minimal Inhibitory Concentration (MIC) of the test compound is determined visually.

Using the methodology described above, examples of the current invention were evaluated for their ability to inhibit the growth of β-lactamase producing bacteria in the presence of a β-lactam antibiotic. Representative results are shown in Table 3, NT=not tested.

TABLE 3
Broad spectrum inhibition of bacterial growth. MIC of example
compounds of the invention in the presence of a fixed amount of a β-lactam
antibiotic (see text for description of antibiotics).

| | | K. pneumoniae | E. cloacae | K. pneumoniae | P. aeruginosa |
|---|---|---|---|---|---|
| Example | E. coli SHV-5 | CTX-M-15 | P99+ | KPC-2 | VIM-2 |
| 1 | 4 | 1 | 0.25 | 0.125 | 8 |
| 2 | 8 | 2 | 0.25 | 0.015 | 8 |
| 3 | 8 | 2 | 4 | 0.12 | >8 |
| 4 | 1 | 0.25 | 2 | 0.06 | >8 |
| 5 | 4 | 1 | 2 | 0.03 | NT |
| 6 | 4 | 1 | 0.25 | 0.03 | NT |
| 7 | 1 | 0.12 | 0.12 | 0.03 | NT |
| 8 | 1 | 0.5 | 0.5 | 0.06 | NT |
| 9 | 4 | 1 | 0.25 | <0.01 | >8 |
| 10 | 1 | 0.25 | 0.5 | 0.01 | >8 |
| 11 | 8 | 1 | 0.25 | <0.01 | 8 |
| 12 | 4 | 1 | 2 | 0.03 | >8 |
| 13 | >8 | >8 | >8 | 0.25 | >8 |
| 14 | >8 | >8 | 0.5 | 0.01 | >8 |
| 15 | 4 | 0.25 | 4 | 0.01 | >8 |
| 16 | 8 | 1 | 0.25 | 0.03 | 4 |
| 17 | >8 | 2 | 1 | 0.12 | >8 |
| 18 | 8 | 0.5 | 0.25 | 0.06 | >8 |
| 19 | 8 | 2 | 2 | 0.12 | >8 |
| 20 | 4 | 1 | 2 | 0.03 | >8 |
| 21 | 4 | 1 | 0.25 | 0.01 | >8 |
| 22 | 8 | 2 | 0.5 | 0.007 | >8 |
| 23 | 8 | 1 | 2 | 0.12 | >8 |
| 24 | 8 | 1 | 1 | 0.12 | >8 |
| 25 | 8 | 1 | 0.5 | 0.03 | >8 |
| 26 | >8 | 4 | 2 | 0.5 | >8 |
| 27 | >8 | 1 | 0.5 | 0.03 | >8 |
| 28 | 8 | 4 | 2 | 2 | >8 |
| 29 | 0.5 | 0.12 | 0.12 | 0.03 | >8 |
| 30 | 0.5 | 0.06 | 0.12 | 0.01 | >8 |
| 31 | 8 | 4 | 2 | 0.25 | >8 |
| 32 | 0.5 | 0.5 | >8 | 4 | >8 |
| 33 | 0.5 | 0.12 | 0.25 | 0.06 | >8 |
| 34 | 0.5 | 0.12 | 0.5 | 0.03 | >8 |
| 35 | 0.5 | 0.25 | 0.25 | 0.03 | >8 |
| 36 | 0.5 | 0.12 | 0.25 | 0.06 | >8 |
| 37 | 2 | 2 | 2 | 0.03 | >8 |
| 38 | 0.5 | 0.25 | 0.5 | 0.03 | v |
| 39 | 0.5 | 0.5 | 2 | 0.03 | >8 |
| 40 | 0.25 | 0.25 | 2 | NT | >8 |
| 41 | 0.5 | 0.25 | 1 | 0.03 | >8 |
| 42 | 0.25 | 0.12 | 0.25 | 0.015 | >8 |
| 43 | 2 | 0.5 | 0.5 | <0.015 | >8 |
| 44 | 1 | 0.25 | 1 | 0.015 | >8 |
| 45 | 0.03 | 0.03 | 0.06 | 0.007 | >8 |
| 46 | 0.06 | 0.03 | 0.06 | 0.007 | >8 |
| 47 | 0.25 | 0.25 | 1 | 0.03 | >8 |
| 48 | >8 | >8 | >8 | 2 | >8 |
| 49 | 2 | 2 | 2 | 0.12 | >8 |
| 50 | >8 | >8 | >8 | >8 | >8 |
| 51 | >8 | >8 | >8 | >8 | >8 |
| 52 | >8 | >8 | >8 | 2 | >8 |
| 53 | NT | NT | NT | NT | NT |
| 54 | 2 | 1 | 1 | 0.03 | >8 |
| 55 | 2 | 2 | 1 | 0.06 | >8 |
| 56 | >8 | 4 | 2 | 0.25 | >8 |
| 57 | 1 | >8 | 4 | 4 | NT |
| 58 | 0.5 | >8 | 2 | 0.5 | NT |
| 59 | 0.5 | >16 | 2 | 1 | NT |
| 60 | 1 | >16 | 2 | 0.5 | NT |
| 61 | 0.25 | 16 | 0.25 | 0.25 | NT |
| 62 | 0.125 | >128 | 0.125 | 0.125 | NT |
| 63 | 0.06 | >16 | 4 | 0.125 | NT |
| 64 | 0.125 | >16 | 4 | 0.125 | NT |
| 65 | 0.25 | >16 | 0.25 | 0.5 | NT |

1. A compound of Formula (I): 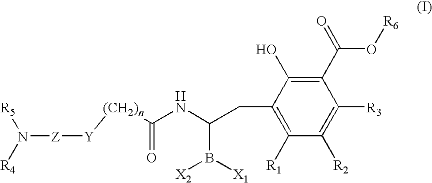
wherein R₁, R₂, and R₃ are independently hydrogen, or selected from the group consisting of hydroxyl, halogen, carboxyl, cyano, thiol, optionally substituted: C1-C5 alkyl, C1-C5 alkoxy, C1-C5 alkenyl, C3-C6 cycloalkyl, C3-C6 heterocyclyl, amino, sulfide, and sulfone;
n is 0, 1, or 2;
Y is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(c) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
R₄ is hydrogen, or selected from the group consisting of:
(a) C1-C5 alkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C1-C5 carbons comprise part of said oxyimino group, imino wherein any of the C1-C5 carbons comprise part of said imino group, amidino wherein any of the C1-C5 carbons comprise part of said amidino group, sulfido, and sulfoxido,
(b) C3-C6 cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfido, and sulfoxido,
(c) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
R₅ is a lone pair of electrons, hydrogen, or selected from the group consisting of:
(a) C1-C5 alkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C1-C5 carbons comprise part of said oxyimino group, imino wherein any of the C1-C5 carbons comprise part of said imino group, amidino wherein any of the C1-C5 carbons comprise part of said amidino group, sulfido, and sulfoxido,
(b) C3-C6 cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfido, and sulfoxido,
(c) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
or R₄ and Y together form a ring of between 5 and 7 atoms where said ring is optionally fused or spiro in relation to the ring system of Y, said ring optionally being partially saturated or aromatic and optionally containing 1-2 additional heteroatoms selected from the group consisting of N, O, S, and a combination thereof;
or R₄ and R₅ together form a ring of between 3 and 7 atoms where said ring is optionally substituted, said ring optionally being saturated, partially unsaturated or aromatic and optionally containing 1-2 additional heteroatoms selected from the group consisting of N, O, S, and a combination thereof;
R₆ is hydrogen or an ester prodrug of the carboxylic acid;
Z is a bond;
or Z is optionally substituted: C1-C4 alkyl, C1-C4 alkoxy, C1-C4 sulfido, C3-C6 cycloalkyl, C3-C6 heterocyclyl where the bond to Y is through a carbon atom of said heterocyclyl ring, heteroaryl where the bond to Y is through a carbon atom of said heteraryl ring, oxyimino, imino, or amidino where the carbon of said oxyimino, imino, or amidino group is attached to Y;
or Z and Y together form a ring of 5-7 atoms where said ring is optionally fused or spiro in relation to the ring system of Y, said ring optionally being partially saturated or aromatic and optionally containing 1-3 heteroatoms selected from the group consisting of N, O, S, and a combination thereof;
or Z and R₄ together form a ring of 4-7 atoms where said ring optionally is saturated, partially unsaturated, or aromatic and optionally contains 1-2 additional heteroatoms selected from the group consisting of N, O, S, and a combination thereof;
X₁ and X₂ are independently hydroxyl, halogen, NR₄R₅, C1-C6 alkoxy, or when taken together X₁ and X₂ form a cyclic boron ester where said chain or ring contains from 2 to 20 carbon atoms and, optionally, 1-3 heteroatoms selected from the group consisting of N, O, S and a combination thereof, or when taken together X₁ and X₂ form a cyclic boron amide where said chain or ring contains from 2 to 20 carbon atoms and, optionally, 1-3 heteroatoms selected from the group consisting of N, O, S, and a combination thereof, or when taken together X₁ and X₂ form a cyclic boron amide-ester where said chain contains from 2-20 carbon atoms and, optionally, 1-3 heteroatoms selected from the group consisting of N, O, S, and a combination thereof, or X₁ is hydroxyl and X₂ is replaced by the ortho-hydroxyl oxygen of the phenyl ring such that a 6-membered ring is formed;
or a salt thereof;
provided that when R₁, R₂, R₃, R₄, R₅, and R₆ are hydrogen, X₁ and X₂ are hydroxyl, n is 0, Y is phenyl, and Z is CH₂ then Z cannot be at the meta-position of the phenyl ring relative to the rest of the molecule.

2. The compound of claim 1 of the formula (II): 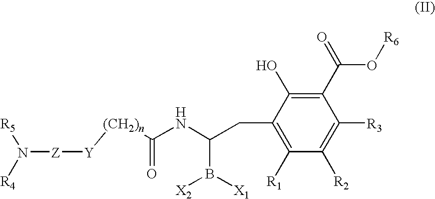
wherein R₁, R₂, and R₃ are independently hydrogen, or selected from the group consisting of hydroxyl, halogen, carboxyl, cyano, thiol, optionally substituted: C1-C5 alkyl, C1-C5 alkoxy, C1-C5 alkenyl, C3-C6 cycloalkyl, C3-C6 heterocyclyl, amino, sulfide, and sulfone;
n is 0, 1, or 2;
Y is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(c) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
R₄ is hydrogen, or selected from the group consisting of:
(a) C1-C5 alkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C1-C5 carbons comprise part of said oxyimino group, imino wherein any of the C1-C5 carbons comprise part of said imino group, amidino wherein any of the C1-C5 carbons comprise part of said amidino group, sulfido, and sulfoxido,
(b) C3-C6 cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfido, and sulfoxido,
(c) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
R₅ is a lone pair of electrons, hydrogen, or selected from the group consisting of:
(a) C1-C5 alkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C1-C5 carbons comprise part of said oxyimino group, imino wherein any of the C1-C5 carbons comprise part of said imino group, amidino wherein any of the C1-C5 carbons comprise part of said amidino group, sulfido, and sulfoxido,
(b) C3-C6 cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfido, and sulfoxido,
(c) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
or R₄ and Y together form a ring of between 5 and 7 atoms where said ring is optionally fused or spiro in relation to the ring system of Y, said ring optionally being partially saturated or aromatic and optionally containing 1-2 additional heteroatoms selected from the group consisting of N, O, S, and a combination thereof;
or R₄ and R₅ together form a ring of between 3 and 7 atoms where said ring is optionally substituted, said ring optionally being saturated, partially unsaturated or aromatic and optionally containing 1-2 additional heteroatoms selected from the group consisting of N, O, S, and a combination thereof;
R₆ is hydrogen or an ester prodrug of the carboxylic acid;
Z is optionally substituted: C1-C4 alkyl, C1-C4 alkoxy, C1-C4 sulfido, C3-C6 cycloalkyl, C3-C6 heterocyclyl where the bond to Y is through a carbon atom of said heterocyclyl ring, heteroaryl where the bond to Y is through a carbon atom of said heteraryl ring, oxyimino, imino, or amidino where the carbon of said oxyimino, imino, or amidino group is attached to Y;
or Z and Y together form a ring of 5-7 atoms where said ring is optionally fused or spiro in relation to the ring system of Y, said ring optionally being partially saturated or aromatic and optionally containing 1-3 heteroatoms selected from the group consisting of N, O, S, and a combination thereof;
or Z and R₄ together form a ring of 4-7 atoms where said ring optionally is saturated, partially unsaturated, or aromatic and optionally contains 1-2 additional heteroatoms selected from the group consisting of N, O, S, and a combination thereof;
X₁ and X₂ are independently hydroxyl, halogen, NR₄R₅, C1-C6 alkoxy, or when taken together X₁ and X₂ form a cyclic boron ester where said chain or ring contains from 2 to 20 carbon atoms and, optionally, 1-3 heteroatoms selected from the group consisting of N, O, S and a combination thereof, or when taken together X₁ and X₂ form a cyclic boron amide where said chain or ring contains from 2 to 20 carbon atoms and, optionally, 1-3 heteroatoms selected from the group consisting of N, O, S, and a combination thereof, or when taken together X₁ and X₂ form a cyclic boron amide-ester where said chain contains from 2-20 carbon atoms and, optionally, 1-3 heteroatoms selected from the group consisting of N, O, S, and a combination thereof, or X₁ is hydroxyl and X₂ is replaced by the ortho-hydroxyl oxygen of the phenyl ring such that a 6-membered ring is formed;
or a salt thereof;
provided that when R₁, R₂, R₃, R₄, R₅, and R₆ are hydrogen, X₁ and X₂ are hydroxyl, n is 0, Y is phenyl, and Z is CH₂ then Z cannot be at the meta-position of the phenyl ring relative to the rest of the molecule.

3. The compound of claim 1, wherein R₁ is hydrogen; R₂ and R₃ are independently hydrogen, or selected from the group consisting of hydroxyl, halogen, carboxyl, cyano, thiol, optionally substituted: C1-C5 alkyl, C1-C5 alkenyl, C1-C5 alkoxy, C3-C6 cycloalkyl, C3-C6 heterocyclyl, amino, sulfide, and sulfone;
n is 0 or 1;
Y is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(c) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
R₄ is hydrogen, or selected from the group consisting of:
(a) C1-C5 alkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C1-C5 carbons comprise part of said oxyimino group, imino wherein any of the C1-C5 carbons comprise part of said imino group, amidino wherein any of the C1-C5 carbons comprise part of said amidino group, sulfido, and sulfoxido,
(b) C3-C6 cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfido, and sulfoxido,
(c) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
R₅ is a lone pair of electrons, hydrogen, or selected from the group consisting of:
(a) C1-C5 alkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C1-C5 carbons comprise part of said oxyimino group, imino wherein any of the C1-C5 carbons comprise part of said imino group, amidino wherein any of the C1-C5 carbons comprise part of said amidino group, sulfido, and sulfoxido,
(b) C3-C6 cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfido, and sulfoxido,
(c) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
or R₄ and Y together form a ring of between 5 and 7 atoms where said ring is optionally fused or spiro in relation to the ring system of Y, said ring optionally being partially saturated or aromatic and optionally containing 1-2 additional heteroatoms selected from the group consisting of N, O, S, and a combination thereof;
or R₄ and R₅ together form a ring of between 3 and 7 atoms where said ring is optionally substituted and optionally is saturated, partially unsaturated or aromatic and optionally contains 1-2 additional heteroatoms selected from the group consisting of N, O, S, and a combination thereof;
R₆ is hydrogen or an ester prodrug of the carboxylic acid;
Z is optionally substituted: C1-C4 alkyl, C1-C4 alkoxy, C1-C4 sulfido, C3-C6 cycloalkyl, C3-C6 heterocyclyl where the bond to Y is through a carbon atom of said heterocyclyl ring, oxyimino, imino, or amidino where the carbon of said oxyimino, imino, or amidino group is attached to Y;
or Z and Y together form a ring of 5-7 atoms where said ring is optionally fused or spiro in relation to the ring system of Y, said ring optionally being partially saturated or aromatic and optionally containing 1-3 heteroatoms selected from the group consisting of N, O, S, and a combination thereof;
or Z and R₄ together form a ring of 4-7 atoms where said ring is optionally saturated, partially unsaturated, or aromatic and optionally contains 1-2 additional heteroatoms selected from the group consisting of N, O, S, and a combination thereof;
X₁ and X₂ are hydroxyl, or when taken together X₁ and X₂ form a cyclic boron ester where said chain or ring contains from 2 to 20 carbon atoms and, optionally, 1-3 heteroatoms selected from the group consisting of N, O, S, and a combination thereof, or X₁ is hydroxyl and X₂ is replaced by the ortho-hydroxyl oxygen of the phenyl ring such that a 6-membered ring is formed;
or a salt thereof;
provided that when R₂, R₃, R₄, R₅, and R₆ are hydrogen, X₁ and X₂ are hydroxyl, n is 0, Y is phenyl, and Z is CH₂ then Z cannot be at the meta-position of the phenyl ring relative to the rest of the molecule.

4. The compound of claim 1, wherein R₁, R₂, R₃, R₄, and R₅ are hydrogen;
R₆ is hydrogen or an ester prodrug of the carboxylic acid;
n is 0 or 1;
Y is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(c) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
Z is optionally substituted: C1-C4 alkyl, C1-C4 alkoxy, C1-C4 sulfido, C3-C6 cycloalkyl, C3-C6 heterocyclyl where the bond to Y is through a carbon atom of said heterocyclyl ring, oxyimino, imino, or amidino where the carbon of the oxyimino, imino, or amidino group is attached to Y;
or Z and Y together form a ring of 5-7 atoms where said ring optionally is partially saturated or aromatic and optionally contains 1-2 additional heteroatoms selected from the group consisting of N, O, S, and a combination thereof;
or Z and R₄ together form a ring of 4-7 atoms where said ring optionally is saturated, partially unsaturated or aromatic and optionally contains 1-2 additional heteroatoms selected from the group consisting of N, O, S, and a combination thereof;
X₁ and X₂ are hydroxyl, or X₁ is hydroxyl and X₂ is replaced by the ortho-hydroxyl oxygen of the phenyl ring such that a 6-membered ring is formed;
or a salt thereof;
provided that when R₆ is hydrogen, X₁ and X₂ are hydroxyl, n is 0, Y is phenyl, and Z is CH₂ then Z cannot be at the meta-position of the phenyl ring relative to the rest of the molecule.

5. The compound of claim 1 of the formula (III): 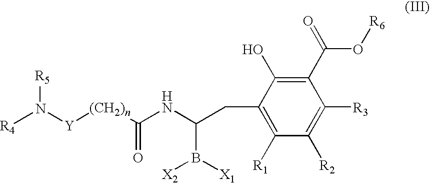
wherein R₁, R₂, and R₃ are independently hydrogen, or selected from the group consisting of hydroxyl, halogen, carboxyl, cyano, thiol, optionally substituted: C1-C5 alkyl, C1-C5 alkoxy, C1-C5 alkenyl, C3-C6 cycloalkyl, C3-C6 heterocyclyl, amino, sulfide, and sulfone;
n is 0, 1, or 2;
Y is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(c) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
R₄ is hydrogen, or selected from the group consisting of:
(a) C1-C5 alkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C1-C5 carbons comprise part of said oxyimino group, imino wherein any of the C1-C5 carbons comprise part of said imino group, amidino wherein any of the C1-C5 carbons comprise part of said amidino group, sulfido, and sulfoxido,
(b) C3-C6 cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfido, and sulfoxido,
(c) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
R₅ is a lone pair of electrons, hydrogen, or selected from the group consisting of:
(a) C1-C5 alkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C1-C5 carbons comprise part of said oxyimino group, imino wherein any of the C1-C5 carbons comprise part of said imino group, amidino wherein any of the C1-C5 carbons comprise part of said amidino group, sulfido, and sulfoxido,
(b) C3-C6 cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfido, and sulfoxido,
(c) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
or R₄ and Y together form a ring of between 3 and 7 atoms where said ring is optionally fused or spiro in relation to the ring system of Y, said ring optionally being saturated, partially saturated or aromatic and optionally containing 1-2 additional heteroatoms selected from the group consisting of N, O, S, and a combination thereof;
or R₄ and R₅ together form a ring of between 3 and 7 atoms where said ring is optionally substituted, said ring optionally being saturated, partially unsaturated or aromatic and optionally containing 1-2 additional heteroatoms selected from the group consisting of N, O, S, and a combination thereof;
R₆ is hydrogen or an ester prodrug of the carboxylic acid;
X₁ and X₂ are independently hydroxyl, halogen, NR₄R₅, C1-C6 alkoxy, or when taken together X₁ and X₂ form a cyclic boron ester where said chain or ring contains from 2 to 20 carbon atoms and, optionally, 1-3 heteroatoms selected from the group consisting of N, O, S and a combination thereof, or when taken together X₁ and X₂ form a cyclic boron amide where said chain or ring contains from 2 to 20 carbon atoms and, optionally, 1-3 heteroatoms selected from the group consisting of N, O, S, and a combination thereof, or when taken together X₁ and X₂ form a cyclic boron amide-ester where said chain contains from 2-20 carbon atoms and, optionally, 1-3 heteroatoms selected from the group consisting of N, O, S, and a combination thereof, or X₁ is hydroxyl and X₂ is replaced by the ortho-hydroxyl oxygen of the phenyl ring such that a 6-membered ring is formed;
or a salt thereof;
provided that when R₁, R₂, R₃, R₄, and R₆ are hydrogen, R₅ is hydrogen or CH₃C(O)—, and X₂ are hydroxyl, n is 1, Y is 4-thiazolyl, then NR₄R₅ cannot be located at the 2-position of the thiazole ring;
further provided that when R₁, R₂, R₃, and R₆ are hydrogen, n is 0, Y is phenyl, and NR₄R₅ is 1-imidazolyl, then NR₄R₅ cannot be located at the 3-position of the phenyl ring relative to the rest of the molecule;
further provided that when R₁, R₂, R₃, and R₆ are hydrogen, n is 0, Y is 5-pyridyl, and NR₄R₅ is 4-morpholinyl, then NR₄R₅ cannot be located at the 2-position of the pyridyl ring.

6. The compound of claim 1, wherein R₁ is hydrogen; R₂ and R₃ are independently hydrogen, or selected from the group consisting of hydroxyl, halogen, carboxyl, cyano, thiol, optionally substituted: C1-C5 alkyl, C1-C5 alkenyl, C1-C5 alkoxy, C3-C6 cycloalkyl, C3-C6 heterocyclyl, amino, sulfide, and sulfone;
n is 0, 1, or 2;
Y is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(c) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
R₄ is hydrogen, or selected from the group consisting of:
(a) C1-C5 alkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C1-C5 carbons comprise part of said oxyimino group, imino wherein any of the C1-C5 carbons comprise part of said imino group, amidino wherein any of the C1-C5 carbons comprise part of said amidino group, sulfido, and sulfoxido,
(b) C3-C6 cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfido, and sulfoxido,
(c) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
R₅ is a lone pair of electrons, hydrogen, or selected from the group consisting of:
(a) C1-C5 alkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the C1-C5 carbons comprise part of said oxyimino group, imino wherein any of the C1-C5 carbons comprise part of said imino group, amidino wherein any of the C1-C5 carbons comprise part of said amidino group, sulfido, and sulfoxido,
(b) C3-C6 cycloalkyl any carbon of which can be substituted with from 0 to 3 substituents selected from the group consisting of hydroxyl, halogen, carboxyl, cyano, oxo, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the cycloalkyl group other than the one attached to the rest of the molecule comprise part of said oxyimino group, imino wherein any of the carbons of the cycloalkyl group other than the one attached to the rest of the molecule comprise part of said imino group, amidino wherein any of the carbons of the cycloalkyl group other than the one attached to the rest of the molecule comprise part of said amidino group, sulfido, and sulfoxido,
(c) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(d) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
or R₄ and Y together form a ring of between 3 and 7 atoms where said ring is optionally fused or spiro in relation to the ring system of Y, said ring optionally being saturated, partially saturated or aromatic and optionally containing 1-2 additional heteroatoms selected from the group consisting of N, O, S, and a combination thereof;
or R₄ and R₅ together form a ring of between 3 and 7 atoms where said ring is optionally substituted and optionally is saturated, partially unsaturated or aromatic and optionally contains 1-2 additional heteroatoms selected from the group consisting of N, O, S, and a combination thereof;
R₆ is hydrogen or an ester prodrug of the carboxylic acid;
X₁ and X₂ are hydroxyl, or when taken together X₁ and X₂ form a cyclic boron ester where said chain or ring contains from 2 to 20 carbon atoms and, optionally, 1-3 heteroatoms selected from the group consisting of N, O, S, and a combination thereof, or X₁ is hydroxyl and X₂ is replaced by the ortho-hydroxyl oxygen of the phenyl ring such that a 6-membered ring is formed;
or a salt thereof;
provided that when R₂, R₃, R₄, and R₆ are hydrogen, R₅ is hydrogen or CH₃C(O)—, X₁ and X₂ are hydroxyl, n is 1, Y is 4-thiazolyl, then NR₄R₅ cannot be located at the 2-position of the thiazole ring;
further provided that when R₂, R₃, and R₆ are hydrogen, n is 0, Y is phenyl, and NR₄R₅ is 1-imidazolyl, then NR₄R₅ cannot be located at the 3-position of the phenyl ring relative to the rest of the molecule;
further provided that when R₂, R₃, and R₆ are hydrogen, n is 0, Y is 5-pyridyl, and NR₄R₅ is 4-morpholinyl, then NR₄R₅ cannot be located at the 2-position of the pyridyl ring.

7. The compound of claim 1, wherein R₁, R₂, R₃, R₄, and R₅ are hydrogen;
R₆ is hydrogen or an ester prodrug of the carboxylic acid;
n is 0 or 1;
Y is selected from the group consisting of:
(a) aryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido,
(b) heteroaryl group substituted with from 0 to 3 substituents selected from the group consisting of hydroxyl, halogen, carboxyl, cyano, thiol, sulfonic acid, sulfate, optionally substituted: alkyl, alkenyl, alkynyl, cycloalkyl, heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino, imino, amidino, sulfido, and sulfoxido, and
(c) heterocyclic group substituted with from 0 to 3 substituents selected from the group consisting of hydroxyl, halogen, carboxyl, cyano, oxo, optionally substituted: heteroaryl, heterocyclyl, alkoxy, cycloalkoxy, heterocyclyloxy, heteroaryloxy, amino, carbonyl, aminocarbonyl, oxycarbonyl, aminosulfonyl, sulfonyl, guanidino, oxyimino wherein any of the carbons of the heterocyclic group other than the one attached to the rest of the molecule comprise part of said oxyimino group, imino wherein any of the carbons of the heterocyclic group other than the one attached to the rest of the molecule comprise part of said imino group, amidino wherein any of the carbons of the heterocyclic group other than the one attached to the rest of the molecule comprise part of said amidino group, sulfido, and sulfoxido;
X₁ and X₂ are hydroxyl, or X₁ is hydroxyl and X₂ is replaced by the ortho-hydroxyl oxygen of the phenyl ring such that a 6-membered ring is formed;
or a salt thereof;
provided that when R₆ is hydrogen, X₁ and X₂ are hydroxyl, n is 1, Y is 4-thiazolyl, then NR₄R₅ cannot be located at the 2-position of the thiazole ring;
further provided that when R₆ is hydrogen, n is 0, Y is phenyl, and NR₄R₅ is 1-imidazolyl, then NR₄R₅ cannot be located at the 3-position of the phenyl ring relative to the rest of the molecule;
further provided that when R₆ is hydrogen, n is 0, Y is 5-pyridyl, and NR₄R₅ is 4-morpholinyl, then NR₄R₅ cannot be located at the 2-position of the pyridyl ring.

8. The compound of claim 1 of the formula (VI): 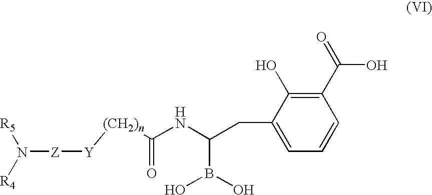
or a salt thereof, wherein
n is 0 or 1;
Y is phenylene or pyridylene;
Z is a bond or —CH₂—;
R₄ is hydrogen, methyl, ethyl, or hydroxyethyl; and
R₅ is hydrogen, acetyl, or C1-C4alkyl optionally substituted with hydroxy or amino.

9. The compound of claim 8 of the formula (VII) 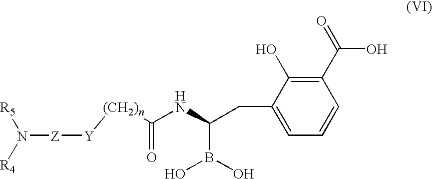
or a salt thereof.

10. A compound selected from the group consisting of

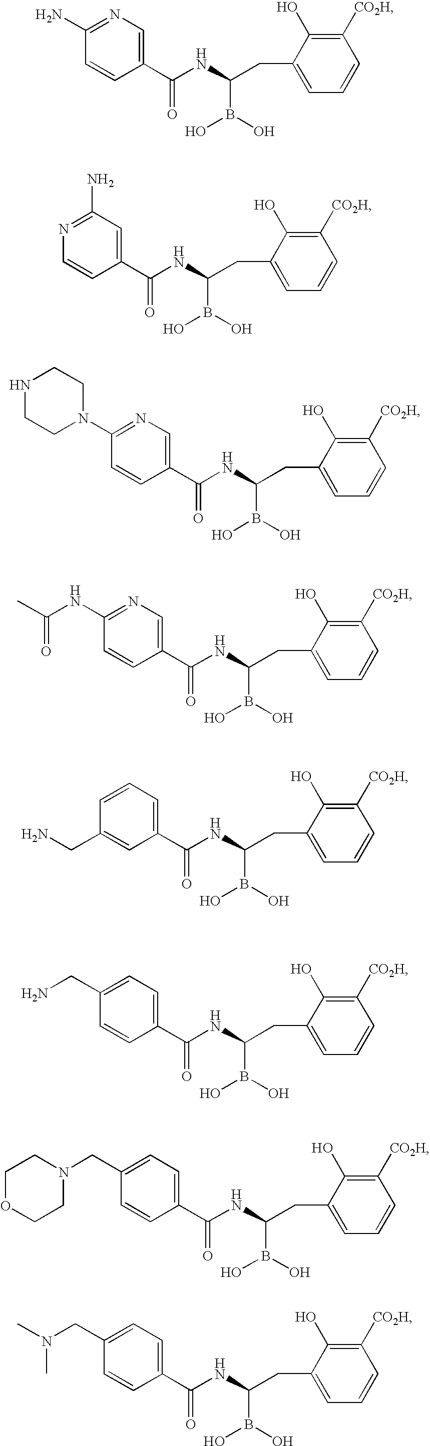

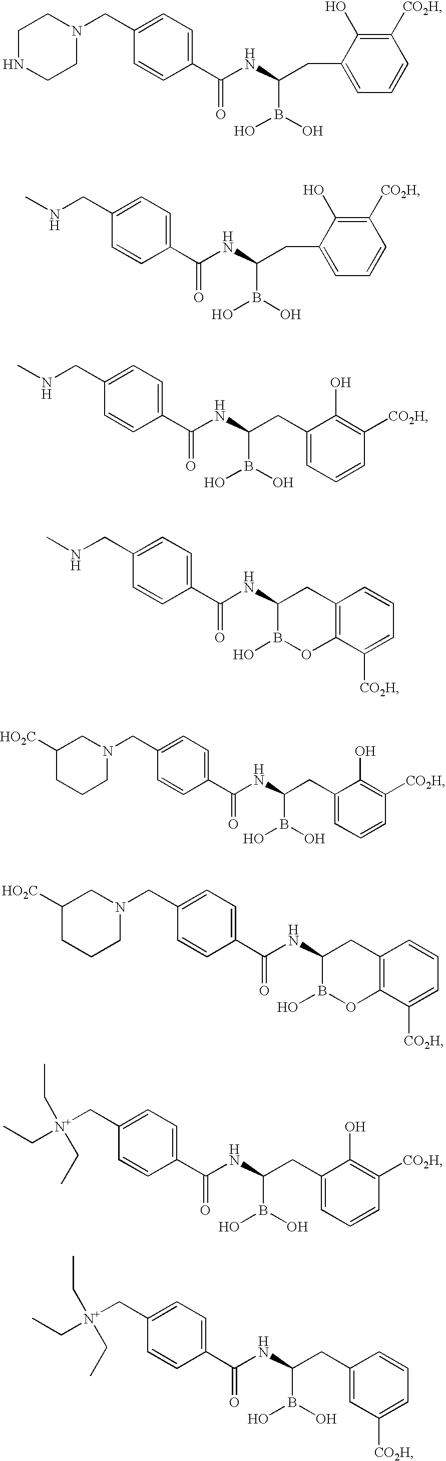

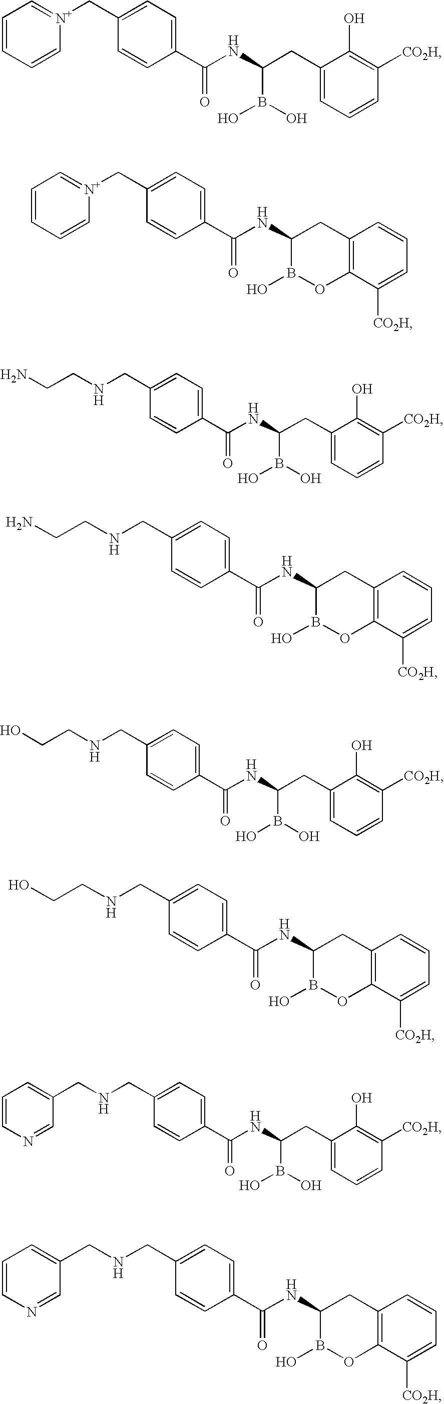

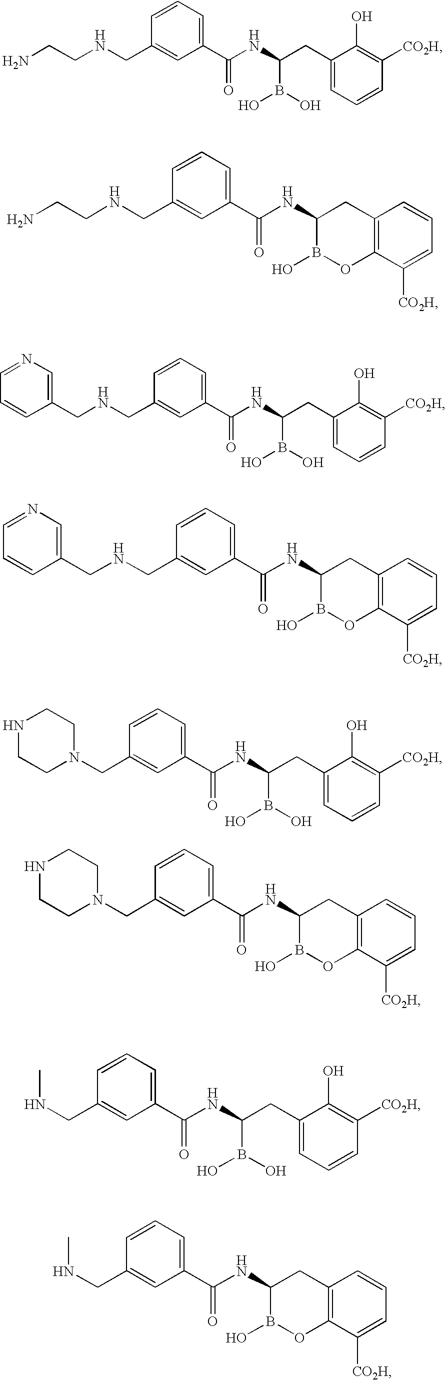

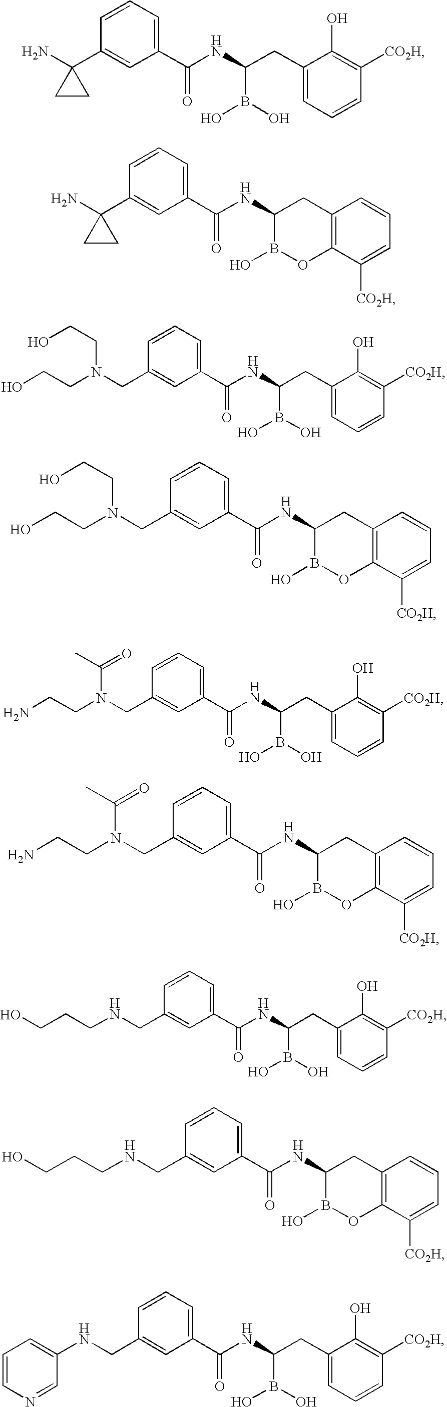

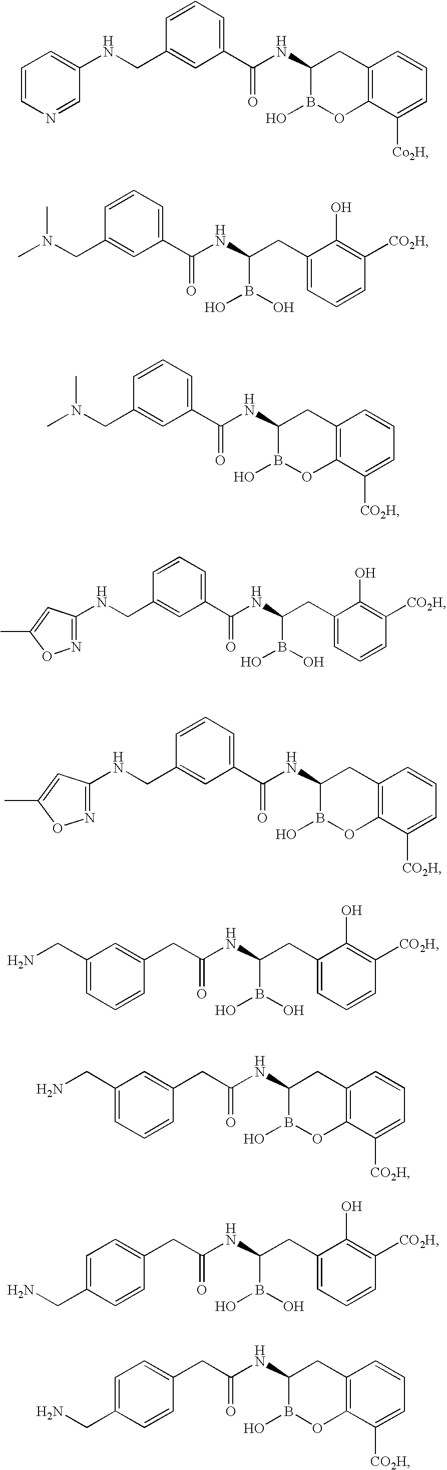

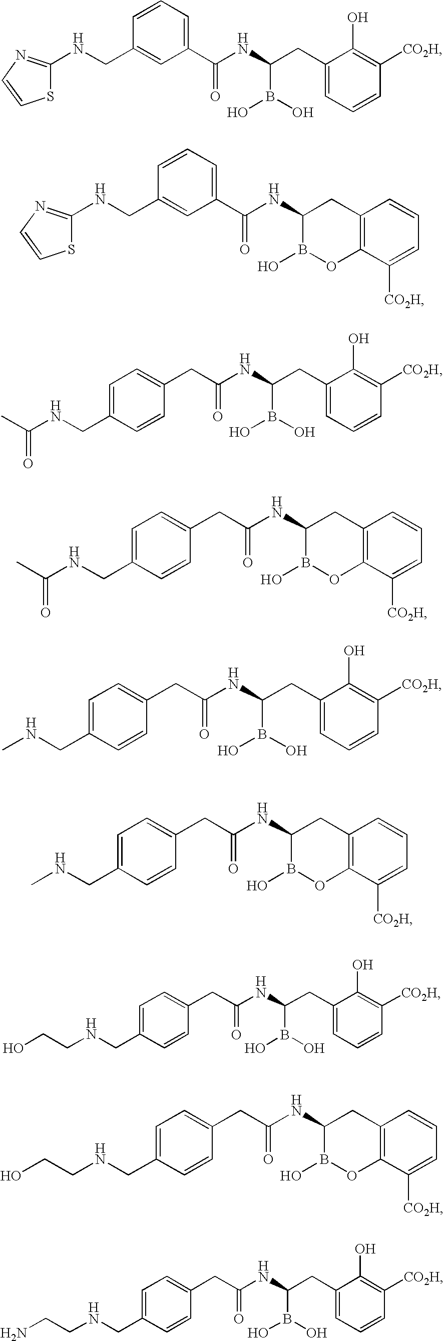

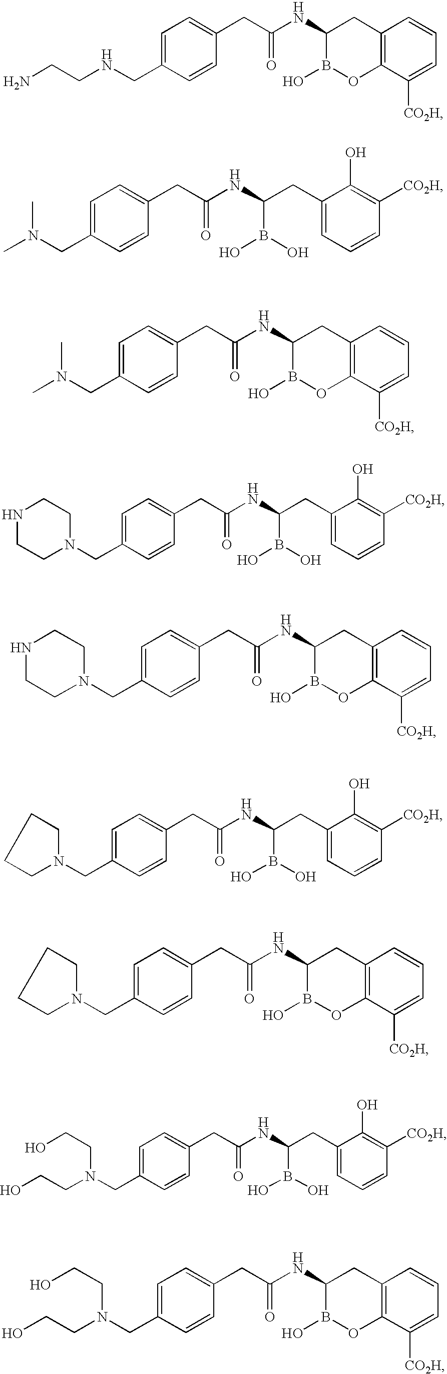

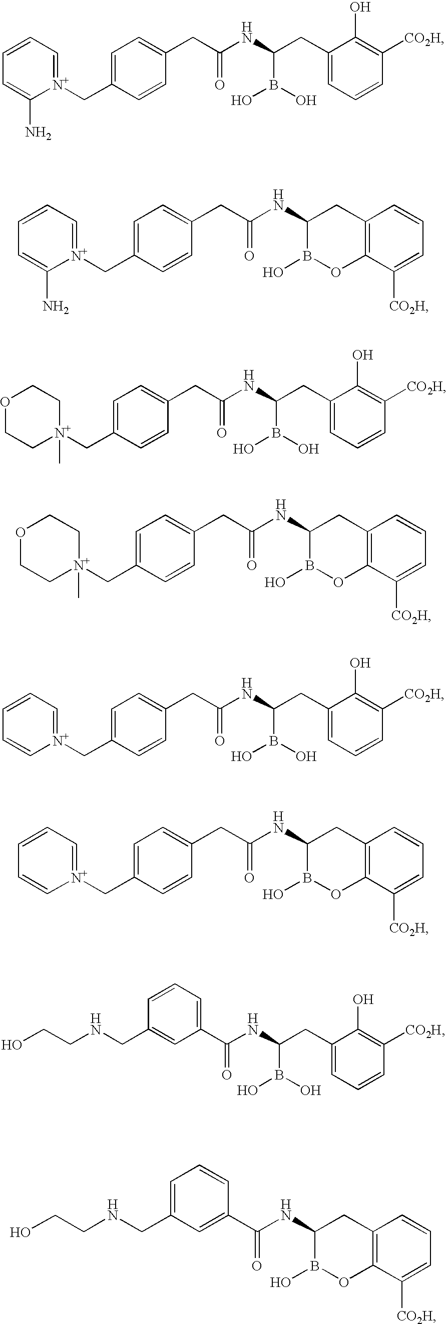

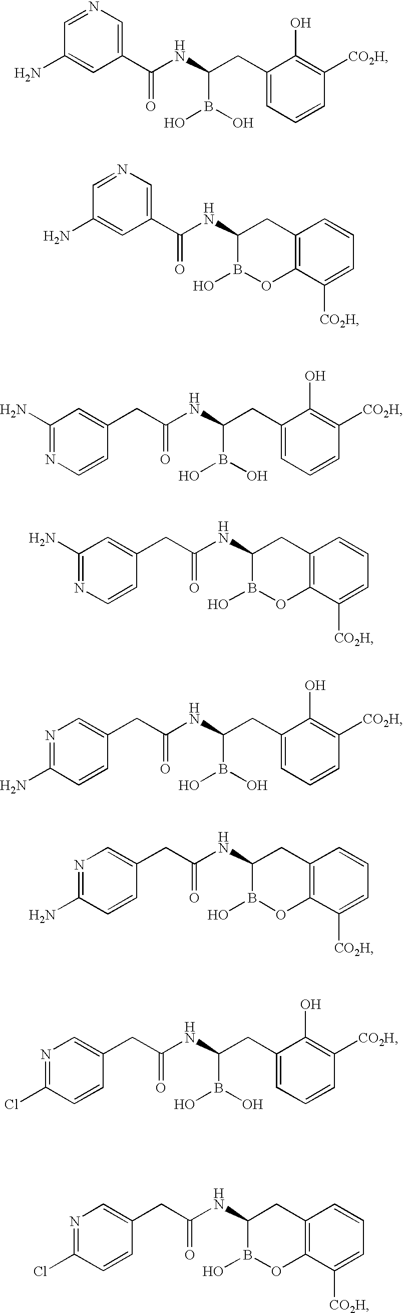

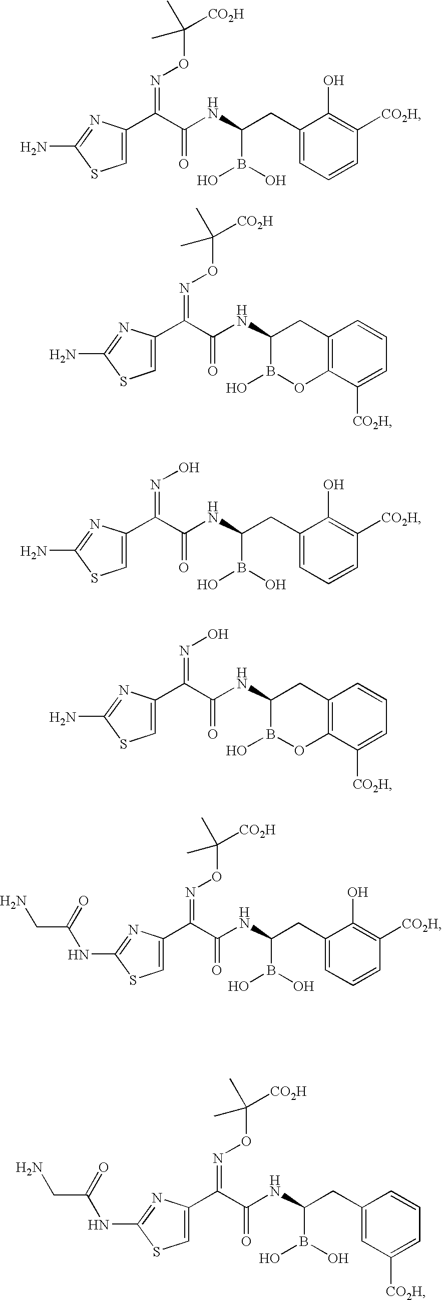

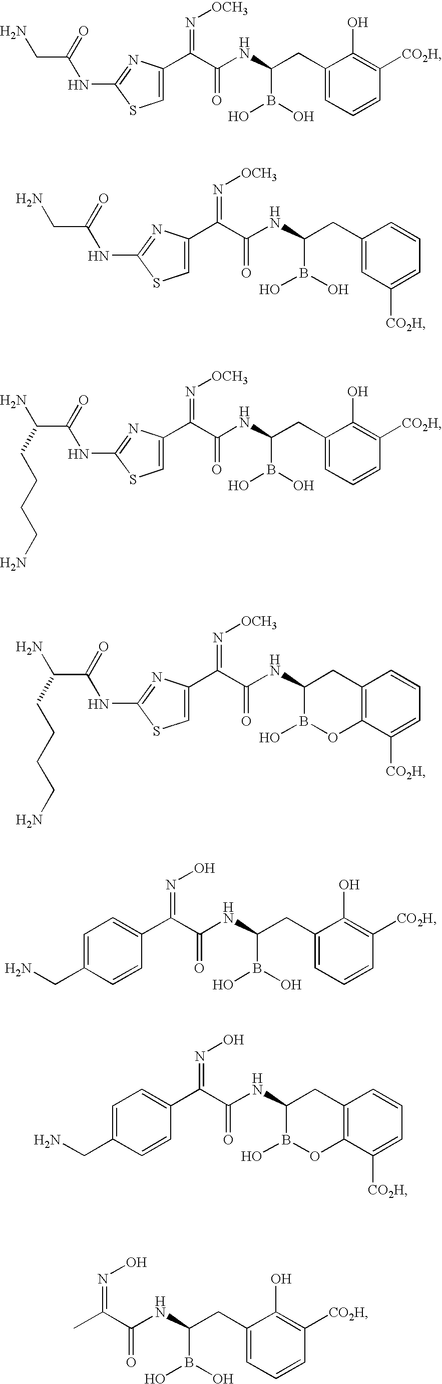

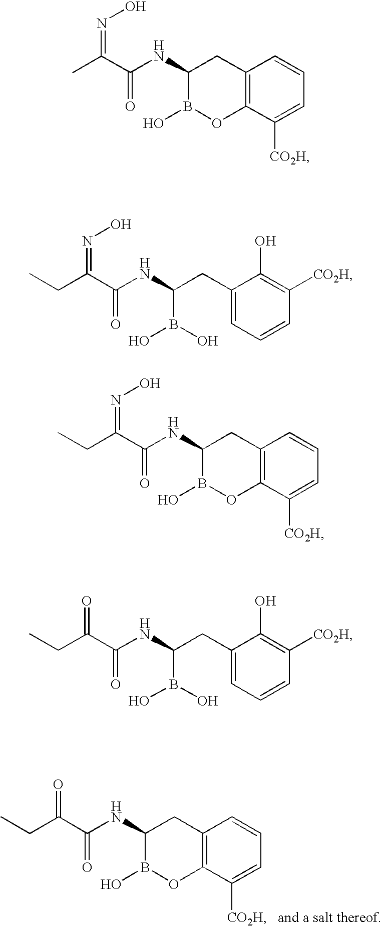

11. A pharmaceutical composition comprising:
(a) one or more compounds of claim 1;
(b) one or more β-lactam antibiotics; and
(c) one or more pharmaceutically acceptable carriers.

12. The pharmaceutical composition of claim 11, wherein the β-lactam antibiotic is a penicillin, cephalosporin, carbapenem, monobactam, bridged monobactam, or combination thereof.

12. The pharmaceutical composition of claim 12, wherein
the penicillin is benzathine penicillin, benzylpenicillin, phenoxymethylpenicillin, procaine penicillin, oxacillin, methicillin, dicloxacillin, flucloxacillin, temocillin, amoxicillin, ampicillin, co-amoxiclav, azlocillin, carbenicillin, ticarcillin, mezlocillin, piperacillin, apalcillin, hetacillin, bacampicillin, sulbenicillin, mecicilam, pevmecillinam, ciclacillin, talapicillin, aspoxicillin, cloxacillin, nafcillin, pivampicillin, or a combination thereof.

13. The pharmaceutical composition of claim 12, wherein the cephalosporin is an anti-MRSA cephalosporin, cephalothin, cephaloridin, cefaclor, cefadroxil, cefamandole, cefazolin, cephalexin, cephradine, ceftizoxime, cefoxitin, cephacetril, cefotiam, cefotaxime, cefsulodin, cefoperazone, ceftizoxime, cefinenoxime, cefinetazole, cephaloglycin, cefonicid, cefodizime, cefpirome, ceftazidime, ceftriaxone, cefpiramide, cefbuperazone, cefozopran, cefepim, cefoselis, cefluprenam, cefuzonam, cefpimizole, cefclidin, cefixime, ceftibuten, cefdinir, cefpodoxime axetil, cefpodoxime proxetil, cefteram pivoxil, cefetamet pivoxil, cefcapene pivoxil, cefditoren pivoxil, cefuroxime, cefuroxime axetil, loracarbacef, latamoxef, FR264205, or a combination thereof.

14. The pharmaceutical composition of claim 12, wherein the carbapenem is an anti-MRSA carbapenem, imipenem, meropenem, ertapenem, faropenem, doripenem, biapenem, panipenem, or a combination thereof.

15. The pharmaceutical composition of claim 12, wherein the monobactam is aztreonam, carumonam, BAL30072, or a combination thereof.

16. A pharmaceutical composition comprising:
(a) one or more compounds of claim 1; and
(b) one or more pharmaceutically acceptable carriers.

17. The pharmaceutical composition of claim 11, comprising more than one beta-lactam antibiotic.

18. A method of treating a bacterial infection in a mammal comprising administering to a mammal in need thereof:
(i) an effective amount of the compound of claim 1; and
(ii) an effective amount of a β-lactam antibiotic.

19. The method of claim 18, wherein the mammal is a human.

20. A method of treating a bacterial infection in a mammal comprising administering to a mammal in need thereof an effective amount of the compound of any one of claims 1-10.

21. The method of claim 20, further comprising contacting the bacterial cell with an effective amount of a β-lactam antibiotic.

22-24. (canceled)
